(19) United States
(12) Patent Application Publication
(10) Pub. No.: US 2024/0140965 A1
(43) Pub. Date: May 2, 2024

(54) TRICYCLIC PESTICIDAL COMPOUNDS

(71) Applicant: BASF SE, Ludwigshafen (DE)

(72) Inventors: Birte Schroeder, Ludwigshafen (DE); Arun Narine, Ludwigshafen (DE); Rizwan Shabbir Shaikh, Navi Mumbai (IN); Pulakesh Maity, Navi Mumbai (IN); Ashokkumar Adisechan, Navi Mumbai (IN)

(21) Appl. No.: 18/280,396

(22) PCT Filed: Mar. 1, 2022
(86) PCT No.: PCT/EP2022/055033

§ 371 Date: Sep. 5, 2023

(30) Foreign Application Priority Data
Mar. 9, 2021 (IN) 202121009819
Apr. 21, 2021 (EP) 21169544.0

(51) Int. Cl. C07D 513/04; A01N 43/90; C07D 498/04

(52) U.S. Cl. CPC C07D 513/04; A01N 43/90; C07D 498/04

(57) ABSTRACT

The invention relates to compounds of formula (I) wherein the variables are as defined in the specification. It also relates to the use of compounds of formula (I) (I); and to agrochemical or veterinary compositions comprising compounds of formula (I). Other objects are seed comprising compounds of formula (I); and methods for controlling invertebrate pests, infestation, or infection by invertebrate pests by application of compounds of formula (I)

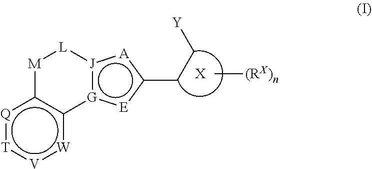

The invention relates to compounds of formula (I) or an agrochemically or verterinarily acceptable salt, stereoisomer, tautomer, or N-oxide thereof

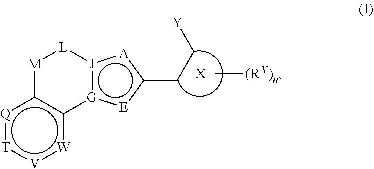

wherein the variables are as defined below. The invention also relates to the use of compounds of formula (I) as an agrochemical pesticide; to pesticidal mixtures comprising a compound of formula (I) and another agrochemically active ingredient; to agrochemical or veterinary compositions comprising a compound of formula (I) or the pesticidal mixture and a liquid or solid carrier; and to seed comprising a compound of formula (I) or the pesticidal mixture. The invention also relates to methods for controlling invertebrate pests, infestation, or infection by invertebrate pests by application of the compounds of formula (I) or the pesticidal mixtures comprising them.

Invertebrate pests and in particular insects, arachnids and nematodes destroy growing and harvested crops and attack wooden dwelling and commercial structures, thereby causing large economic loss to the food supply and to property. Accordingly, there is an ongoing need for new agents for combating invertebrate pests.

WO2020083662A2 discloses tricyclic pesticidal compounds.

Due to the ability of target pests to develop resistance to pesticidally active agents, there is an ongoing need to identify further compounds, which are suitable for combating invertebrate pests such as insects, arachnids and nematodes. Furthermore, there is a need for new compounds having a high pesticidal activity and showing a broad activity spectrum against a large number of different invertebrate pests, especially against difficult to control insects, arachnids and nematodes. There is furthermore a need to find compounds that display a higher efficacy as compared with known pesticides, which reduces the application rates and costs for the applicant, and decreases the environmental effects on soil and ground water. There is also a need to provide pesticidal compounds that are environmentally friendly, i.e. for example have a moderate half-life time and thus do not accumulate in the soil, ground or surface waters, and/or which have no or a low toxicity towards beneficial organisms.

It is therefore an object of the present invention to identify and provide compounds, which exhibit a high pesticidal activity, have a broad activity spectrum against invertebrate pests and are environmentally friendly.

It has been found that these objects can be achieved by substituted tricyclic compounds of formula (I) as depicted and defined below, including their stereoisomers, their salts, in particular their agriculturally or veterinarily acceptable salts, their tautomers and their N-oxides.

Therefore, the invention provides in a first aspect compounds of formula (I), or an agrochemically or veterinarily acceptable salt, stereoisomer, tautomer, or N-oxide thereof

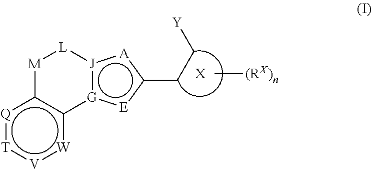

wherein the variables in formula (I) have the following meaning
A is CH, N, or NH;
E is N, O, S, NR^{E}, or CR^{E};
G, J are independently C or N, provided that only one of E or G is N; the group -M-L- is selected from O—CR^{L1}R^{L2}, S—CR^{L1}R^{L2}, CR^{M1}R^{M2}—O, or CR^{M1}R^{M2}—S;
Q is N or CR^{Q};
T is N or CR^{T};
V is N or CR^{v};
W is N or CR^{W};
X is phenyl, or a 5- or 6-membered heteroaryl;
Y is SR^{Y1}, S(O)R^{Y1}, S(O)₂R^{Y1}, S(═O)(═NR^{Y2})R^{Y1}, or S(═NR^{Y2})(═NR^{Y3})R^{Y1};
   R^{E}, R^{Q}, R^{T}, R^{V}, and R^{W} are independently H, halogen, N₃, CN, NO₂, SCN, SF₅;
      C₁-C₆-alkyl, C₁-C₆-alkoxy, C₂-C₆-alkenyl, tri-C₁-C₆-alkysilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxyx-C₁-C₄-alkyl, which groups are halogenated or non-halogenated,
      C(═O)OR¹, NR²R³, C(═O)NR²R³, C(═O)R⁴, SO₂NR²R³, S(═O)ₘR⁵, OR⁶, SR⁶, or CH₂R⁶;
      phenyl, which is unsubstituted or substituted with one or more, same or different substituents R¹¹;
   R¹ H;
      C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆ -cycloalkoxy-C₁-C₄-alkyl, which groups are halogenated or non-halogenated; or
      phenyl, which is unsubstituted, or substituted with one or more, same or different substituents R¹¹;
      R¹¹ is halogen, OH, CN, NO₂, SCN, SF₅;
         C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆ -cycloalkyl, C₃-C₆ -cycloalkoxy, C₃-C₆-cycloalkyl-C₁-C₄-alkyl, C₃-C₆-cycloalkoxy-C₁-C₄-alkyl, which groups are halogenated or non-halogenated;
   R² is H;
      C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted, or substituted with one or more, same or different substitutent selected from halogen, CN and HO.
      C(═O)R²¹, C(═O)OR²¹, C(═O)NR²¹, C₁-C₆-alkylen-CN, or CH₂R⁶; or
      phenyl, which is unsubstituted or substituted with one or more, same or different substituents R¹¹;
      R²¹ is H;
         C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl;
         C₃-C₆-cycloalkyl-C₁-C₄-alkyl, C₃-C₆-cycloalkoxy-C₁-C₄ alkyl, phenyl, or a saturated, partially-, or fully unsaturated 5- or 6-membered heterocycle, wherein the cyclic moieties are unsubstituted or substituted with one or more, same or different substituents R¹¹;
   R³ is H;
      C₁-C₆-alkyl, C₂-C₆-alkenyl, C₂-C₆-alkynyl, C₁-C₆-aloxy-C₁-C₄-alkyl, C₃-C₆-cycloalkyl, C₃-C₆-cycloalkoxy-C₁-C₄-alkyl, C₃-C₆-cycloalkoxy-C₁-C₄-alkyl, which groups are halogenated or non-halogenated;
      C₁-C₆-alkylen-CN, or CH₂R⁶;
      phenyl, which is unsubstituted or substituted with one or more, same or different substituents R¹¹; or
      NR²R³ may also form an N-bound, saturated 3- to 8-membered heterocycle, which in addition to the nitrogen atom may have 1 or 2 further heteroatoms or heteroatom moieties selected from O, S(═O)ₘ, NH, and N—C₁-C₆-alkyl, and wherein the N-bound heterocycle is unsubstituted or substituted with one or more, same or different substituents selected from halogen, C₁-C₄-alkyl, C₁-C₄-haloalkyl, C₁-C₄-alkoxy and C₁-C₄-haloalkoxy;
   R⁴ is H, C₁-C₄-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆-cycloalkoxy-C₁-C₄-alkyl, which are unsubstituted or substituted with one or more, same of different substituents selected from halogen, CN, and OH;
      CH₂R⁶, or phenyl, which is unsubstituted, or substituted with one or more, same or different substituents R¹¹;
   R⁵ is C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆-cycloalkoxy-C₁-C₄-alkyl, which groups are halogenated or non-halogenated;
      C₁-C₆-alkylen-NR²R³, C₁-C₆-alkylen-CN, CH₂R⁶; or
      phenyl, which is unsubstituted, or substituted with one or more, same or different substituents R¹¹;
   R⁶ is phenyl, which is unsubstituted or substituted with one or more, same or different substituents R¹¹;
   each R^{L1}, R^{L2}, R^{M1}, R^{M2} is independently H, halogen, OH;
      C₁-C₄-alkyl, C₃-C₆-cycloalkyl, C₃-C₆-cycloalkyl-C₁-C₂-alkyl, C₂-C₄-alkenyl, C₃-C₆-cycloalkenyl, C₂-C₄-alkynyl, C₁-C₄-alkoxy, which groups are unsubstituted, or substituted with one or more, same or different substituents selected from halogen and CN,
      phenyl, which is unsubstituted, or substituted with one or more, same or different substituents selected from halogen, CN, C₁-C₃-alkyl, C₁-C₃-haloalkyl, C₁-C₃-alkoxy, and C₁-C₃-haloalkoxy; or
      R^{L1} and R^{L2}, or R^{M1}and R^{M2} form together with the carbon atom they are bonded to a group C═O, C═S, C═NR⁷R⁸, or C═NOR⁷;
   each R⁷, R⁸ is independently H;
      C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted, or substituted with one or more, same or different substitutent selected from halogen, CN and OH.
      benzyl or phenyl, which is unsubstituted or substituted with one or more, same or different substituents R¹¹, or
      NR⁷R⁸ may also form an N-bound, saturated 3- to 6-membered heterocycle, which in addition to the nitrogen atom may have 1 or 2 further heteroatoms or heteroatom moieties selected from O, S(═O)ₘ, NH, and N—C₁-C₆-alkyl, and wherein the N-bound heterocycle is unsubstituted or substituted with one or more, same or different substituents selected from halogen, C₁-C₄-alkyl, C₁-C₄-haloalkyl, C₁-C₄-alkoxy and C₁-C₄-haloalkoxy;
   each R^{X} is independently halogen, N₃, OH, CN, NO₂, SCN, SF₅;
      C₁-C₆-alkyl, C₁-C₆-alkoxy, C₂-C₆-alkenyl, tri-C₁-C₆-alkylsilyl, C₂-C₆-alkynyl, C₁-C₆-alkoxy-C₁-C₄-alkoxy, C₁-C₄-alkoxy-C₁-C₄-alkoxy, C₃-C₆-cycloalkyl, C₃-C₆-cycloalkoxy, C₃-C₆-cycloalkoxy-C₁-C₄-alkyl, C₃-C₆-cycloalkoxy-C₁-C₄-alkyl, which groups are usubstituted or substituted with CN or halogen;
      C(═O)OR¹, NR²R³, C(═O)NR²R³, C(═O)R⁴, SO₂NR²R³, S(═O)ₘR¹, OR⁶, SR⁶, CH₂R⁶, OC(═O)R⁴, NR³C(═O)R⁴, OC(═O)OR¹, OC(═O)NR²R³, OC(═O)SR¹, OC(═S)NR²R³, OC(═S)SR¹, ONR²R³, ON═CR¹R⁴, N═CR¹R⁴, NNR², NC(═O)R⁹, SC(═O)SR¹, SC(═O)NR²R³, C(═S)R⁶, C(═S)OR⁴, C(═NR²)R⁴, C(R^{10a})═N—O(R^{10b});
      phenyl, which is unsubstituted or substituted with one or more, same or different substituents R¹¹;
      a 5- or 6-membered saturated, partially unsaturated, or fully unsaturated heterocyclic ring, wherein said heterocyclic ring comprises one or more, same or different heteroatoms O, N, or S, and is unsubstituted, or substituted with one or more, same or different substituents R³¹, and wherein said N- and S-atoms are independently oxidized, or non-oxidized; or
      a group of formula (S)

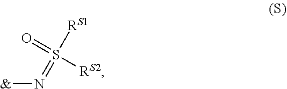

wherein each R^{S1}, R^{S2} is independently selected from C₁-C₆-alkyl, C₃-C₆-cycloalkyl, C₂-C₆-alkenyl, C₃-C₆-cycloalkenyl, C₂-C₆-alkynyl, C₃-C₆-cycloalkyl-C₁-C₃-alkyl, which groups are unsubstituted, or halogenated;
a 3- to 6-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said N- and S-atoms are independently oxidized, or non-oxidized;
phenyl, which is unsubstituted, or substituted with one or more, same or different substituents selected from halogen, C₁-C₃-alkyl, C₁-C₃-alkoxy, C₁-C₃-haloalkyl, and C₁-C₃-haloalkoxy;
or two substituents R^{S1}, R^{S2} form, together with the sulfur atom to which they are bound, a 5- or 6- membered saturated, partially unsaturated, or fully unsaturated heterocycle, which heterocycle is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said heterocycle comprises no, one or more, same or different heteroatoms O, N, or S in addition to the sulfur atom to which R^{S1} and R^{S2} are bound to;
R³¹ is halogen, N₃, OH, CN, NO₂, SCN, SF₅;
   C₁-C₆-alkyl, C₁-C₆-haloalkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₁-C₆-alkoxycarbonyl, C₃-C₆-cycloalkyl;
   C₃-C₆-cycloalkoxy, C₃-C₆-cycloalkyl-C₁-C₄-alkyl, C₃-C₆-cycloalkoxy-C₁-C₄ alkyl, phenyl, or a saturated, partially-, or fully unsaturated 5- or 6-membered heterocycle, wherein the cyclic moieties are unsubstituted or substituted with one or more, same or different substituents R¹¹; or
   two geminal substituents R³¹ form together with the atom to which they are bound a group ═O or ═S;
each R⁹ is independently C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with one or more, same or different substituents selected from CN and halogen;
each R^{10a} is independently H, CN, OH, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen;
   phenyl or benzyl, wherein the phenyl ring is unsubstituted or substituted with one or more, same or different substituents R¹¹;
each R^{10b} is independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with one or more, same or different substituents selected from halogen and CN; phenyl or benzyl, wherein the phenyl ring is unsubstituted or substituted with one or more, same or different substitutents R¹¹;
R³¹ is halogen, N₃, OH, CN, NO₂, SCN, SF₅;
   C₁-C₆-alkyl, C₁-C₆-haloalkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₁-C₆-alkoxycarbonyl, C₃-C₆-cycloalkyl;
   C₃-C₆-cycloalkoxy, C₃-C₆-cycloalkyl-C₁-C₄-alkyl, C₃-C₆-cycloalkoxy-C₁-C₄ alkyl, phenyl, or a saturated, partially-, or fully unsaturated 5- or 6-membered heterocycle, wherein the cyclic moieties are unsubstituted or substituted with one or more, same or different substituents R¹¹; or
   two geminal substituents R³¹ form together with the atom to which they are bound a group ═O or ═S.
each R^{Y1}, R^{Y2}, R^{Y3} is independently selected from H, C₁-C₆-alkyl, C₃-C₆-cycloalkyl, C₂-C₆-alkenyl, C₃-C₆-cycloalkenyl, C₂-C₆-alkynyl, C₃-C₆-cycloalkyl-C₁-C₃-alkyl, which groups are unsubstituted, or substituted with one or more, same or different substituents selected from halogen and CN;
   a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents selected from halogen, CN, C₁-C₃-alkyl, C₁-C₃-alkoxy, C₁-C₃-haloalkyl, C₁-C₃-haloalkoxy, and wherein said N- and S-atoms are independently oxidized, or non-oxidized;
   phenyl, which is unsubstituted, or substituted with one or more, same or different substituents selected from halogen, CN, C₁-C₃-alkyl, C₁-C₃-alkoxy, C₁-C₃-haloalkyl, C₁-C₃-haloalkoxy;
   or two substituents selected from R^{Y1}, R^{Y2}, R^{Y3}, form, together with the S- or N-atoms to which they are bound, a 5- or 6- membered saturated, partially unsaturated, or fully unsaturated heterocycle, which heterocycle is unsubstituted, or substituted with one or more, same or different substituents selected from halogen, CN, C₁-C₃-alkyl, C₁-C₃-alkoxy, C₁-C₃-haloalkyl, C₁-C₃-haloalkox, and wherein said heterocycle comprises no, one or more, same or different heteroatoms O, N, or S in addition to the S- and N-atoms to which the two substituents selected from R^{Y1}, R^{Y2}, and R^{3Y}, are bound to;
      the index n is 0, 1, 2, 3, or 4 if X is phenyl or a 6-membered heteroaryl;
      or 0, 1, 2, or 3 if X is a 5-membered heteroaryl; and
      the index m is 0, 1, or 2.

The tricyclic compounds of the formula (I), and their agriculturally acceptable salts are highly active against animal pest, i.e. harmful arthropodes and nematodes, especially against insects and acaridae which are difficult to control by other means.

Moreover, the present invention relates to and includes the following embodiments:
compositions comprising at least one compound of formula (I) as defined above;
agricultural and veterinary compositions comprising an amount of at least one compound of formula (I) or an enantiomer, diasteromer or salt thereof as defined above;
methods for combating invertebrate pests, infestation, or infection by invertebrate pests, which method comprises contacting said pest or its food supply, habitat or breeding grounds with a pesticidally effective amount of at least one compound of formula (I) as defined above or a composition thereof;
methods for controlling invertebrate pests, infestation, or infection by invertebrate pests, which method comprises contacting said pest or its food supply, habitat or breeding grounds with a pesticidally effective amount of at least one compound of formula (I) as defined above or a composition comprising at least one compound of formula (I);
methods for preventing or protecting against invertebrate pests comprising contacting the invertebrate pests, or their food supply, habitat or breeding grounds with substituted imidazolium compounds of the general formula (I) as defined above or a composition comprising at least one compound of formula (I) as defined above or a composition comprising at least one compound of formula (I);
methods for protecting crops, plants, plant propagation material and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with a pesticidally effective amount of at least one compound of formula (I) as defined above or a composition comprising at least one compound of formula (I);
non-therapeutic methods for treating animals infested or infected by parasites or preventing animals of getting infected or infested by parasites or protecting animals against infestation or infection by parasites which comprises orally, topically or parenterally administering or applying to the animals a parasiticidally effective amount of a compound of formula (I) as defined above or a composition comprising at least one compound of formula (I);
methods for treating, controlling, preventing or protecting animals against infestation or infection by parasites by administering or applying orally, topically or parenterally to the animals a substituted imidazolium compound of the general formula (I) as defined above or a composition comprising at least one compound of formula (I);
seed comprising a compound of formula (I) as defined above, in an amount of from 0.1 g to 10 kg per 100 kg of seed;
the use of the compounds of formula (I) as defined above for protecting growing plants or plant propagation material from attack or infestation by invertebrate pests;
the use of compounds of formula (I) or the enantiomers, diastereomers or veterinary acceptable salts thereof for combating parasites in and on animals;
a process for the preparation of a veterinary composition for treating, controlling, preventing or protecting animals against infestation or infection by parasites which comprises adding a parasiticidally effective amount of an compound of formula (I) or the enantiomers, diastereomers and/or veterinary acceptable salt thereof to a carrier composition suitable for veterinary use;
the use of a compound of formula (I) or the enantiomers, diastereomers and/or veterinary acceptable salt thereof for the preparation of a medicament for treating, controlling, preventing or protecting animals against infestation or infection by parasites.

All the compounds of formula (I) if applicable their stereoisomers, their tautomers, their salts or their N-oxides as well as compositions thereof are particularly useful for controlling invertebrate pests, in particular for controlling arthropods and nematodes and especially insects. Therefore, the invention relates to the use of a compound of formula (I) as an agrochemical pesticide, preferably for combating or controlling invertebrate pests, in particular invertebrate pests of the group of insects, arachnids or nematodes.

The term “compound(s) according to the invention” or “compound(s) of formula (I)” as used in the present invention refers to and comprises the compound(s) as defined herein and/or stereoisomer(s), salt(s), tautomer(s) or N-oxide(s) thereof. The term “compound(s) of the present invention” is to be understood as equivalent to the term “compound(s) according to the invention”, therefore also comprising stereoisomer(s), salt(s), tautomer(s) or N-oxide(s) of compounds of formula (I).

The term “composition(s) according to the invention” or “composition(s) of the present invention” encompasses composition(s) comprising at least one compound of formula (I) according to the invention as defined above, therefore also including a stereoisomer, an agriculturally or veterinary acceptable salt, tautomer or an N-oxide of the compounds of formula (I).

The compounds of the present invention may be amorphous or may exist in one or more different crystalline states (polymorphs) or modifications which may have a different macroscopic properties such as stability or show different biological properties such as activities. The present invention includes both amorphous and crystalline compounds of the formula (I), mixtures of different crystalline states or modifications of the respective compound (I), as well as amorphous or crystalline salts thereof.

The compounds of the formula (I) have one or, depending on the substitution pattern, more centers of chirality, in which case they are present as mixtures of enantiomers or diastereomers. The invention provides both the single pure enantiomers or pure diastereomers of the compounds of formula (I), and their mixtures and the use according to the invention of the pure enantiomers or pure diastereomers of the compound of formula (I) or its mixtures. Suitable compounds of the formula (I) also include all possible geometrical stereoisomers (cis/trans isomers) and mixtures thereof. Cis/trans isomers may be present with respect to an alkene, carbon-nitrogen double-bond or amide group. The term “stereoisomer(s)” encompasses both optical isomers, such as enantiomers or diastereomers, the latter existing due to more than one center of chirality in the molecule, as well as geometrical isomers (cis/trans isomers). The present invention relates to every possible stereoisomer of the compounds of formula (I), i.e. to single enantiomers or diastereomers, as well as to mixtures thereof.

Depending on the substitution pattern, the compounds of the formula (I) may be present in the form of their tautomers. Hence the invention also relates to the tautomers of the formula (I) and the stereoisomers, salts, tautomers and N-oxides of said tautomers.

Salts of the compounds of the formula (I) are preferably agriculturally and/or veterinary acceptable salts. They can be formed in a customary method, e.g. by reacting the compound with an acid of the anion in question if the compound of formula (I) has a basic functionality or by reacting an acidic compound of formula (I) with a suitable base.

Suitable agriculturally or veterinary useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formulae I with an acid of the corresponding anion, preferably of hydrochloric acid, hydrobromic acid, sulfuric acid, phosphoric acid or nitric acid.

The term “N-oxide” includes any compound of the present invention which has at least one tertiary nitrogen atom that is oxidized to an N-oxide moiety.

The organic moieties groups mentioned in the above definitions of the variables are—like the term halogen—collective terms for individual listings of the individual group members. The prefix Cₙ-Cₘ indicates in each case the possible number of carbon atoms in the group. “Halogen” will be taken to mean F, Cl, Br, and I, preferably F.

The term “substituted with”, e.g. as used in “partially, or fully substituted with” means that one or more, e.g. 1, 2, 3, 4 or 5 or all of the hydrogen atoms of a given radical have been replaced by one or more, same or different substituents, such as a halogen, in particular F.

Accordingly, for substituted cyclic moieties, e.g. 1-cyanocyclopropyl, one or more of the hydrogen atoms of the cyclic moiety may be replaced by one or more, same or different substituents.

Likewise, the term “halogenated” means that one or more, e.g. 1, 2, 3, 4, or 5 or all of the hydrogen atoms of a given radical have been replaced by one or more, same or different halogen atoms, such as F.

The term “Cₙ-Cₘ-alkyl” as used herein (and also in Cₙ-Cₘ-alkylamino, di-Cₙ-Cₘ-alkylamino, Cₙ-Cₘ-alkylaminocarbonyl, di-(Cₙ-Cₘ-alkylamino)carbonyl, Cₙ-Cₘ-alkylthio, Cₙ-Cₘ-alkylsulfinyl and Cₙ-Cₘ-alkylsulfonyl) refers to a branched or unbranched saturated hydrocarbon group having n to m, e.g. 1 to 10 carbon atoms, preferably 1 to 6 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl.

The term “Cₙ-Cₘ-haloalkyl” as used herein (and also in Cₙ-Cₘ-haloalkylsulfinyl and Cₙ-Cₘ-haloalkylsulfonyl) refers to a straight-chain or branched alkyl group having n to m carbon atoms, e.g. 1 to 10 in particular 1 to 6 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like. The term C₁-C₁₀-haloalkyl in particular comprises C₁-C₂-fluoroalkyl, which is synonym with methyl or ethyl, wherein 1, 2, 3, 4 or 5 hydrogen atoms are substituted with fluorine atoms, such as fluoromethyl, difluoromethyl, trifluoromethyl, 1-fluoroethyl, 2-fluoroethyl, 2,2-difluoroethyl, 2,2,2-trifluoroethyl and pentafluoromethyl.

Similarly, “Cₙ-Cₘ-alkoxy” and “Cₙ-Cₘ-alkylthio” (or Cₙ-Cₘ-alkylsulfenyl, respectively) refer to straight-chain or branched alkyl groups having n to m carbon atoms, e.g. 1 to 10, in particular 1 to 6 or 1 to 4 carbon atoms (as mentioned above) bonded through oxygen (or sulfur linkages, respectively) at any bond in the alkyl group. Examples include C₁-C₄-alkoxy such as methoxy, ethoxy, propoxy, isopropoxy, butoxy, sec-butoxy, isobutoxy and tert-butoxy, further C₁-C₄-alkylthio such as methylthio, ethylthio, propylthio, isopropylthio, and n-butylthio.

Accordingly, the terms “Cₙ-Cₘ-haloalkoxy” and “Cₙ-Cₘ-haloalkylthio” (or Cₙ-Cₘ-haloalkyl-sulfenyl, respectively) refer to straight-chain or branched alkyl groups having n to m carbon atoms, e.g. 1 to 10, in particular 1 to 6 or 1 to 4 carbon atoms (as mentioned above) bonded through oxygen or sulfur linkages, respectively, at any bond in the alkyl group, where some or all of the hydrogen atoms in these groups may be replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy, further C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like. Similarly, the terms C₁-C₂-fluoroalkoxy and C₁-C₂-fluoroalkylthio refer to C₁-C₂-fluoroalkyl which is bound to the remainder of the molecule via an oxygen atom or a sulfur atom, respectively.

The term “C₂-Cₘ-alkenyl” as used herein intends a branched or unbranched unsaturated hydrocarbon group having 2 to m, e.g. 2 to 10 or 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “C₂-Cₘ-alkynyl” as used herein refers to a branched or unbranched unsaturated hydrocarbon group having 2 to m, e.g. 2 to 10 or 2 to 6 carbon atoms and containing at least one triple bond, such as ethynyl, propynyl, 1-butynyl, 2-butynyl, and the like.

The term “Cₙ-Cₘ-alkoxy-Cₙ-Cₘ-alkyl” as used herein refers to alkyl having n to m carbon atoms, e.g. like specific examples mentioned above, wherein one hydrogen atom of the alkyl radical is replaced by an Cₙ-Cₘ-alkoxy group; wherein the value of n and m of the alkoxy group are independently chosen from that of the alkyl group.

The suffix “-carbonyl” in a group or “C(═O)” denotes in each case that the group is bound to the remainder of the molecule via a carbonyl C═O group. This is the case e.g. in alkylcarbonyl, haloalkylcarbonyl, aminocarbonyl, alkylaminocarbonyl, dialkylaminocarbonyl, alkoxycarbonyl, haloalkoxycarbonyl.

The term “aryl” as used herein refers to a mono-, bi- or tricyclic aromatic hydrocarbon radical such as phenyl or naphthyl, in particular phenyl (also referred as to C₆H₅ as subsitituent). The term “C₃-Cₘ-cycloalkyl” as used herein refers to a monocyclic ring of 3- to m-membered saturated cycloaliphatic radicals, e.g. cyclopropyl, cyclobutyl, cyclopentyl, cyclohexyl, cycloheptyl, cyclooctyl and cyclodecyl.

The term “alkylcycloalkyl” denotes as well as the term “alkyl which may be substituted with cycloalkyl” an alkyl group which is substituted with a cycloalkyl ring, wherein alkyl and cycloakyl are as herein defined.

The term “cycloalkylalkyl” denotes as well as the term “cycloalkyl which may be substituted with alkyl” a cycloalkyl ring which is substituted with an alkyl group, wherein alkyl and cycloakyl are as herein defined.

The term “alkylcycloalkylalkyl” denotes as well as the term “alkylcycloalkyl which may be substituted with alkyl” an alkylcycloalkyl group which is substituted with an alkyl, wherein alkyl and alkylcycloakyl are as herein defined.

The term “ C₃-Cₘ-cycloalkenyl” as used herein refers to a monocyclic ring of 3- to m-membered partially unsaturated cycloaliphatic radicals.

The term “cycloalkylcycloalkyl” denotes as well as the term “cycloalkyl which may be substituted with cycloalkyl” a cycloalkyl substitution on another cycloalkyl ring, wherein each cycloalkyl ring independently has from 3 to 7 carbon atom ring members and the cycloalkyls are linked through one single bond or have one common carbon atom. Examples of cycloalkylcycloalkyl include cyclopropylcyclopropyl (e.g. 1,1′-bicyclopropyl-2-yl), cyclohexylcyclohexyl wherein the two rings are linked through one single common carbon atom (e.g. 1,1′-bicyclohexyl-2-yl), cyclohexylcyclopentyl wherein the two rings are linked through one single bond (e.g. 4-cyclopentylcyclohexyl) and their different stereoisomers such as (1R,2S)-1,1′-bicyclopropyl-2-yl and (1R,2R)-1,1′-bicyclopropyl-2-yl. The term “carbocycle” or “carbocyclyl” includes, unless otherwise indicated, in general a 3- to 12-membered, preferably a 3- to 8-membered or a 5- to 8-membered, more preferably a 5- or 6-membered mono-cyclic, ring comprising 3 to 12, preferably 3 to 8 or 5 to 8, more preferably 5 or 6 carbon atoms.

The carbocyclic radicals may be saturated, partially unsaturated, or fully unsaturated. Preferably, the term “carbocycle” covers cycloalkyl and cycloalkenyl groups as defined above, for example cyclopropane, cyclobutane, cyclopentane and cyclohexane rings. When it is referred to “fully unsaturated” carbocycles, this term also includes “aromatic” carbocycles. In certain preferred embodiments, a fully unsaturated carbocycle is an aromatic carbocycle as defined below, preferably a 6-membered aromatic carbocycle.

The term “heteroaryl” or “aromatic heterocycle” or “aromatic heterocyclic ring” includes monocyclic 5- or 6-membered heteroaromatic radicals comprising as ring members 1, 2, 3 or 4 heteroatoms selected from N, O and S. Examples of 5- or 6-membered heteroaromatic radicals include pyridyl, i.e. 2-, 3-, or 4-pyridyl, pyrimidinyl, i.e. 2-, 4- or 5-pyrimidinyl, pyrazinyl, pyridazinyl, i.e. 3- or 4-pyridazinyl, thienyl, i.e. 2- or 3-thienyl, furyl, i.e. 2-or 3-furyl, pyrrolyl, i.e. 2- or 3-pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5-isothiazolyl, pyrazolyl, i.e. 1-, 3-, 4- or 5-pyrazolyl, i.e. 1-, 2-, 4- or 5-imidazolyl, oxadiazolyl, e.g. 2- or 5-[1,3,4]oxadiazolyl, 4- or 5-(1,2,3-oxa-diazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl. The term “heteroaryl” also includes bicyclic 8 to 10-membered heteroaromatic radicals comprising as ring members 1, 2 or 3 heteroatoms selected from N, O and S, wherein a 5- or 6-membered heteroaromatic ring is fused to a phenyl ring or to a 5- or 6-membered heteroaromatic radical. Examples of a 5- or 6-membered heteroaromatic ring fused to a phenyl ring or to a 5- or 6-membered heteroaromatic radical include benzofuranyl, benzothienyl, indolyl, indazolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2-d]pyrimidyl or pyridoimidazolyl and the like. These fused heteroaryl radicals may be bonded to the remainder of the molecule via any ring atom of 5- or 6-membered heteroaromatic ring or via a carbon atom of the fused phenyl moiety.

The terms “heterocycle”, “heterocyclyl” or “heterocyclic ring” includes, unless otherwise indicated, in general 3- to 12-membered, preferably 3- to 8-membered, 3- to 7-membered, or 5- to 8-membered, more preferably 5- or 6-membered, in particular 6-membered monocyclic heterocyclic radicals. The heterocyclic radicals may be saturated, partially unsaturated, or fully unsaturated. As used in this context, the term “fully unsaturated” also includes “aromatic”. In a preferred embodiment, a fully unsaturated heterocycle is thus an aromatic heterocycle, preferably a 5- or 6-membered aromatic heterocycle comprising one or more, e.g. 1, 2, 3, or 4, preferably 1, 2, or 3 heteroatoms selected from N, 0 and S as ring members. Examples of aromatic heterocycles are provided above in connection with the definition of “heteroaryl”. Unless otherwise indicated, “heteroaryls” are thus covered by the term “heterocycles”. The heterocyclic nonaromatic radicals usually comprise 1, 2, 3, 4 or 5, preferably 1, 2 or 3 heteroatoms selected from N, O and S as ring members, where S-atoms as ring members may be present as S, SO or SO₂. Examples of 5- or 6-membered heterocyclic radicals comprise saturated or unsaturated, non-aromatic heterocyclic rings, such as oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxothiolanyl, dihydrothienyl, S-oxodihydrothienyl, S-dioxodihydrothienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S.oxothiopyranyl, S-dioxothiopyranyl, dihydrothiopyranyl, S-oxodihydrothiopyranyl, S-dioxodihydrothiopyranyl, tetrahydrothiopyranyl, S-oxotetrahydrothiopyranyl, S-dioxotetrahydrothiopyranyl, morpholinyl, thiomorpholinyl, S-oxothiomorpholinyl, S-dioxothiomorpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-onyl and the like.

The erms “alkylene”, “alkenylene”, and “alkynylene” refer to alkyl, alkenyl, and alkynyl as defined above, respectively, which are bonded to the remainder of the molecule, via two atoms, preferably via two carbon atoms, of the respective group, so that they represent a linker between two moieties of the molecule. In particular, the term “alkylene” may refer to alkyl chains such as CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂, CH₂CH₂CH₂CH₂CH₂CH₂, and CH₂CH₂CH₂CH₂CH₂CH₂CH₂. Similarly, “alkenylene” and “alkynylene” may refer to alkenyl and alkynyl chains, respectively.

The term “5- to 6-membered carbocyclic ring” as used herein refers to cyclopentane and cyclohexane rings.

Examples of 5- or 6-membered saturated heterocyclic rings include: 2-tetrahydrofuranyl, 3-tetrahydrofuranyl, 2-tetrahydrothienyl, 3-tetrahydrothienyl, 2-pyrrolidinyl, 3-pyrrolidinyl, 3-pyrazolidinyl, 4-pyrazolidinyl, 5-pyrazolidinyl, 2-imidazolidinyl, 4-imidazolidinyl, 2-oxazolidinyl, 4-oxazolidinyl, 5-oxazolidinyl, 3-isoxazolidinyl, 4-isoxazolidinyl, 5-isoxazolidinyl, 2-thiazolidinyl, 4-thiazolidinyl, 5-thiazolidinyl, 3-isothiazolidinyl, 4-isothiazolidinyl, 5-isothiazolidinyl, 1,2,4-oxadiazolidin-3-yl, 1,2,4-oxadiazolidin 5 yl, 1,2,4-thiadiazolidin-3-yl, 1,2,4-thiadiazolidin-5-yl, 1,2,4-triazolidin-3-yl,-1,3,4-oxadiazolidin-2-yl, 1,3,4-thiadiazolidin-2-yl, 1,3,4-triazolidin-2-yl, 2-tetrahydropyranyl, 4-tetrahydropyranyl, 1,3-dioxan-5-yl, 1,4-dioxan-2-yl, 2-piperidinyl, 3-piperidinyl, 4-piperidinyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl, 2-morpholinyl, 3-morpholinyl, 2-thiomorpholinyl, 3-thiomorpholinyl, 1-oxothiomorpholin-2-yl, 1-oxothiomorpholin-3-yl, 1,1-dioxothiomorpholin-2-yl, 1,1-dioxothiomorpholin-3-yl.

Examples of 5- or 6-membered partially unsaturated heterocyclyl or heterocyclic rings include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin 3 yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3 dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl.

Examples of 5- or 6-membered fully unsaturated heterocyclic (heteroaryl) or heteroaromatic rings are: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

A “C₂-Cₘ-alkylene” is divalent branched or preferably unbranched saturated aliphatic chain having 2 to m, e.g. 2 to 7 carbon atoms, for example CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂, CH₂CH₂CH₂CH₂CH₂CH₂, and CH₂CH₂CH₂CH₂CH₂CH₂CH₂.

### PREPARATION METHODS

The compounds of formula (I) can be prepared by standard methods of organic chemistry. If certain derivatives cannot be prepared by the processes outlined below, they can be obtained by derivatization of other compounds of formula (I) that are accessible by these methods. The variables in the following formulae are—unless otherwise provided—as defined for formula (I).

Compounds of formula (1) falling under the definition of formula (I), wherein M is O, may be prepared by a series of synthesis steps as displayed under General Scheme 1

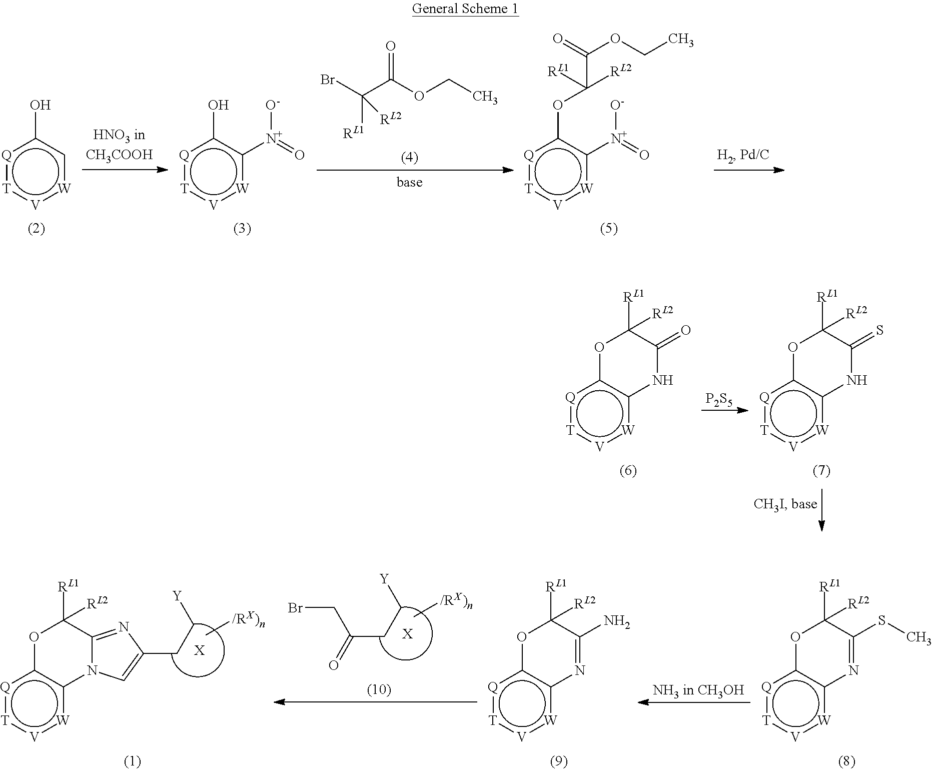

In a first step, compounds of formula (2) may be reacted in an electrophilic nitration reaction with HNO₃ in the presence of an acid like CH₃COOH or H₂SO₄ to afford compounds of formula (3). The reaction is typically carried in the acid as a solvent.

Compounds of formula (3) may then be reacted with compounds of formula (4) in the presence of a base in an inert solvent at a temperature of from 50 to 120° C. Typical solvents include aliphatic hydrocarbons, preferably an aliphatic C₅-C₁₆-hydrocarbon, more preferably a C₅-C₁₆-alkane, or C₅-C₁₆-cycloalkane, such as pentane, hexane, cyclohexane, or petrol ether; aromatic hydrocarbons, preferably an aromatic C₆-C₁₀-hydrocarbons, such as benzene, toluene, o-, m-, and p-xylene; nitriles, preferably C₁-C₆-nitriles, such as CH₃CN, and CH₃CH₂CN; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and CH₃C(O)C(CH₃)₃ (MTBK); moreover dimethyl sulfoxide (DMSO), sulfolane, and mixtures thereof.

Suitable bases are, in general, inorganic bases, such as alkali metal and alkaline earth metal hydroxides, such as LiOH, NaOH, KOH, and Ca(OH)₂; alkali metal and alkaline earth metal oxides, such as Li₂O, Na₂O, CaO, and MgO; alkali metal and alkaline earth metal hydrides, such as LiH, NaH, KH and CaH₂; alkali metal and alkaline earth metal carbonates, such as Li₂CO₃, K₂CO₃ and CaCO₃; alkali metal bicarbonates, such as NaHCO₃; alkali metal phosphates, such as NaH₂PO₄, Na₂HPO₄, Na₃PO₄, KH₂PO₄, K₂HPO₄, K₄PO₄; organic bases, for example secondary amines, such as pyrrolidine; tertiary amines, such as diisopropylethylamine, trimethylamine, triethylamine, triisopropylamine and N-methylpiperidine, imidazol, pyridine; substituted pyridines, such as collidine, lutidine and 4-dimethylaminopyridine, and also polycyclic amides, such as 1,8-diazabicycloundec-7-ene (DBU), 1,4-Diazabicyclo[2.2.2]octane (DABCO); alkali metal salts of secondary amines, such as alkali diisopropylamide, alkali bis(trimethylsilyl)amide, alkali tetramethylpiperidene;
alcoholates, such as alkali methanolate, alkali ethanolate, alkali isopropanolate, alkali tertbutanolate; alkali metal—alkyl, and alkali metal—aryl salts, such as n-butyl lithium, tert-butyl lithium, phenyl lithium. Mixtures of the aforementioned bases are also possible. The bases are generally employed in catalytic amounts; however, they can also be used in equimolar amounts, in excess or, if appropriate, as solvent.

Compounds of formula (5) may then be hydrated by reaction with H₂ in the presence of a catalyst such as Pd on C in a suitable inert solvent at a temperature of from 25 to 80° C. to yield compounds of formula (6). Suitable solvents include aliphatic hydrocarbons, preferably an aliphatic C₅-C₁₆-hydrocarbon, more preferably a C₅-C₁₆-alkane, or C₅-C₁₆-cycloalkane, such as pentane, hexane, cyclohexane, or petrol ether; aromatic hydrocarbons, preferably an aromatic C₆-C₁₀-hydrocarbons, such as benzene, toluene, o-, m-, and p-xylene; ethers, preferably C₁-C₆-cycloalkyl ethers, C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, CH₃OC(CH₃)₃ (MTBE), CH₃OCH₃ (DME), CH₃OCH₂CH₂OCH₃, dioxane, anisole, and tetrahydrofurane (THF); alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; water, and mixtures thereof.

Compounds of formula (6) may then be reacted with a thiation reagent like P₂S₅ or Lawesson's reagent at a temperature of from 80 to 150° C. in an inert solvent to yield compounds of formula (7). Suitable solvents include aliphatic hydrocarbons, preferably an aliphatic C₅-C₁₆-hydrocarbon, more preferably a C₅-C₁₆-alkane, or C₅-C₁₆-cycloalkane, such as pentane, hexane, cyclohexane, or petrol ether; aromatic hydrocarbons, preferably an aromatic C₆-C₁₀-hydrocarbons, such as benzene, toluene, o-, m-, and p-xylene.

Compounds of formula (7) may then be methylated by reaction with CH₃I in the presence of a base in an inert solvent to yield compounds of formula (8). The reaction is typically carried out at a temperature of from 10 to 40° C.

Suitable solvents are aliphatic hydrocarbons, preferably an aliphatic C₅-C₁₆-hydrocarbon, more preferably a C₅-C₁₆-alkane, or C₅-C₁₆-cycloalkane, such as pentane, hexane, cyclohexane, or petrol ether; aromatic hydrocarbons, preferably an aromatic C₆-C₁₀-hydrocarbons, such as benzene, toluene, o-, m-, and p-xylene; halogenated hydrocarbons, preferably halogenated aliphatic C₁-C₆-alkanes, or halogenated aromatic C₆-C₁₀-hydrocarbons, such as CH₂Cl₂, CHCl₃, CCl₄, CH₂ClCH₂Cl, CCl₃CH₃, CHCl₂CH₂Cl, CCl₂CCl₂, or chlorobenzene; ethers, preferably C₁-C₆-cycloalkyl ethers, C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, CH₃OCH₂CH₂OCH₃, dioxane, anisole, and THF; nitriles, preferably C₁-C₆-nitriles, such as CH₃CN, and CH₃CH₂CN; and mixtures thereof.

Suitable bases are, in general, inorganic bases, such as alkali metal and alkaline earth metal hydroxides, such as LiOH, NaOH, KOH, and Ca(OH)₂; alkali metal and alkaline earth metal oxides, such as Li₂O, Na₂O, CaO, and MgO; alkali metal and alkaline earth metal carbonates, such as Li₂CO₃, K₂SO₃ and CaCO₃; alkali metal bicarbonates, such as NaHCO₃; alkali metal phosphates, such as NaH₂PO₄, Na₂HPO₄, Na₃PO₄, KH₂PO₄, K₂HPO₄, K₄PO₄; organic bases, for example secondary amines, such as pyrrolidine; tertiary amines, such as diisopropylethylamine, trimethylamine, triethylamine, triisopropylamine and N-methylpiperidine, imidazol, pyridine; substituted pyridines, such as collidine, lutidine and 4-dimethylaminopyridine, and also polycyclic amides, such as DBU, DABCO and mixtures thereof.

Compounds of formula (8) may then be reacted with NH₃ in an inert solvent to yield compounds of formula (9). The reaction is typically carried out at a temperature of from 0 to 50° C. Suitable solvents include aliphatic hydrocarbons, preferably an aliphatic C₅-C₁₆-hydrocarbon, more preferably a C₅-C₁₆-alkane, or C₅-C₁₆-cycloalkane, such as pentane, hexane, cyclohexane, or petrol ether; aromatic hydrocarbons, preferably an aromatic C₆-C₁₀-hydrocarbons, such as benzene, toluene, o-, m-, and p-xylene; ethers, preferably C₁-C₆-cycloalkyl ethers, C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, CH₃OCH₂CH₂OCH₃, dioxane, anisole, and THF; nitriles, preferably C₁-C₆-nitriles, such as CH₃CN, and CH₃CH₂CN; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and CH₃C(O)C(CH₃)₃ (MTBK); alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; amides and urea derivatives, preferably dimethyl formamide (DMF), N-methyl-2-pyrrolidone (NMP), dimethyl acetamide (DMA), 1,3-dimethyl-2-imidazolidinone (DMI), 1,3-dimethyl-3,4,5,6-tetrahydro-2(1H)-pyrimidinone (DMPU), hexamethylphosphamide (HMPA); DMSO, sulfolane, water and mixtures thereof.

Compounds of formula (9) may then be reacted with compounds of formula (10) to yield compounds of formula (1). Reactions of this type have been described in EP3257853A1 and WO2018206479. The reaction is typically carried out under elevated temperatures of from 50-160° C. in an inert solvent, optionally in the presence of a molecular sieve. Suitable solvents are aliphatic hydrocarbons, such as pentane, hexane, cyclohexane, or petrol ether; aromatic hydrocarbons, such as benzene, toluene, o-, m-, and p-xylene; halogenated hydrocarbons, or halogenated aromatic C₆-C₁₀-hydrocarbons, such as CH₂Cl₂, CHCl₃, CCl₄, CH₂ClCH₂Cl, CCl₃CH₃, CHCl₂CH₂Cl, CCl₂CCl₂, or chlorobenzene; ethers, such as CH₃CH₂OCH₂CH₃, (CH₃)₂CHOCH(CH₃)₂, MTBE, DME, CH₃OCH₂CH₂OCH₃, CH₃OC(CH₃)₂CH₂CH₃, dioxane, anisole, 2-methyltetrahydrofuran, THF, and diethylene glycol; nitriles, such as CH₃CN, and CH₃CH₂CN; alcohols, such as CH₃OH, CH₃CH₂OH, CH₃CH₂CH₂OH, CH₃CH(OH)CH₃, CH₃(CH₂)₃OH, and C(CH₃)₃OH, CH₂(OH)CH₂(OH), CH₃CH(OH)CH₂OH; amides and urea derivatives, such as DMF, NMP, dimethyl acetamide (DMA), DMI, DMPU, HMPA; moreover DMSO, sulfolane, and water. Mixtures of the above solvents are also possible.

The reaction may be carried out in the presence of a catalyst, such as an acid or a base, preferably a base. Suitable bases are, in general, inorganic bases, such as LiOH, NaOH, KOH, and Ca(OH)₂; alkali metal and alkaline earth metal oxides, such as Li₂O, Na₂O, CaO, and MgO; alkali metal and alkaline earth metal hydrides, such as LiH, NaH, KH and CaH₂; alkali metal and alkaline earth metal carbonates, such as Li₂CO₃, K₂CO₃ and CaCO₃; alkali metal bicarbonates, such as NaHCO₃; organic bases, such as pyrrolidine; tertiary amines, such as diisopropylethylamine, trimethylamine, triethylamine, triisopropylamine and N-methylpiperidine, imidazol, pyridine; substituted pyridines, such as collidine, lutidine and 4-dimethylaminopyridine, and polycyclic amides and amidines, such as DBU, DABCO; alkali metal salts of secondary amines, such as alkali diisopropylamide, alkali bis(trimethylsilyl)amide, alkali tetramethylpiperidene; alcoholates, such as alkali methanolate, alkali ethanolate, alkali isopropanolate, alkali tertbutanolate; alkali metal—alkyl, and alkali metal—aryl salts, such as n-butyl lithium, tert-butyl lithium, phenyl lithium. Mixtures of the aforementioned bases are also possible. The bases are generally employed in catalytic amounts; however, they can also be used in equimolar amounts, in excess or, if appropriate, as solvent.

Compounds (9) and compounds (10) are typically reacted with one another in equimolar amounts. In terms of yield, it may be advantageous to employ an excess of compounds (2). Compounds of formula (10) are accessible by a variety of synthetic methods, such as described in EP Application Number 2115353132.2, EP Application Number 20168197.0, WO 2017/167832 A1, and WO 2018/206479A1.

Compounds of formula (11) falling under the definition of formula (I), wherein L is O, may be prepared by a series of synthesis steps as displayed under General Scheme 2.

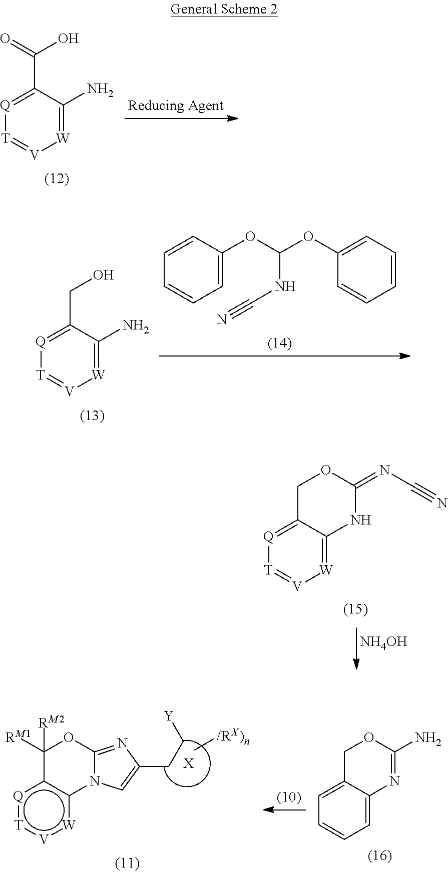

Compounds of formula (12) may be reacted with a reducing agent in the presence of an inert solvent at a temperature of from −5 to 50° C. to yield compounds of formula (13). Suitable solvents are aliphatic hydrocarbons, preferably an aliphatic C₅-C₁₆-hydrocarbon, more preferably a C₅-C₁₆-alkane, or C₅-C₁₆-cycloalkane, such as pentane, hexane, cyclohexane, or petrol ether; aromatic hydrocarbons, preferably an aromatic C₆-C₁₀-hydrocarbons, such as benzene, toluene, o-, m-, and p-xylene; ethers, preferably C₁-C₆-cycloalkyl ethers, C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, CH₃OCH₂CH₂OCH₃, dioxane, anisole, and THF; alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; water and mixtures thereof. Suitable reducing agents include BH₃, LiBH₄, AlH₃, or LiAlH₄ and mixtures thereof. The reducing agent is typically employed in an excess of compounds of formula (12).

Compounds of formula (13) may then be reacted with compounds of formula (14) to yield compounds of formula (15). Compounds of formula (14) are commercially available or may be prepared as described in Webb and Lee et al., Journal of Heterocyclic Chemistry (1982), 19(5), 1205-6. The reaction is typically carried out at a temperature of 50 to 120° C. in an inert solvent.

Suitable solvents are aliphatic hydrocarbons, preferably an aliphatic C₅-C₁₆-hydrocarbon, more preferably a C₅-C₁₆-alkane, or C₅-C₁₆-cycloalkane, such as pentane, hexane, cyclohexane, or petrol ether; aromatic hydrocarbons, preferably an aromatic C₆-C₁₀-hydrocarbons, such as benzene, toluene, o-, m-, and p-xylene; halogenated hydrocarbons, preferably halogenated aliphatic C₁-C₆-alkanes, or halogenated aromatic C₆-C₁₀-hydrocarbons, such as CH₂Cl₂, CHCl₃, CCl₄, CH₂ClCH₂Cl, CCl₃CH₃, CHCl₂CH₂Cl, CCl₂CCl₂, or chlorobenzene; ethers, preferably C₁-C₆-cycloalkyl ethers, C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, CH₃OCH₂CH₂OCH₃, dioxane, anisole, and THF; alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; and mixtures thereof. Typically an excess of compounds of formula (14) is employed relative to the amount of compounds of formula (13).

Compounds of formula (15) may subsequently be incubated at elevated temperatures such as 50 to 100° C. in an inert solvent and optionally in the presence of a base to yield compounds of formula (16). Reactions of this type have been described in Garratt, Peter et al, Tetrahedron (1989), 45(3), 829-34. Suitable solvents include aliphatic hydrocarbons, preferably an aliphatic C₅-C₁₆-hydrocarbon, more preferably a C₅-C₁₆-alkane, or C₅-C₁₆-cycloalkane, such as pentane, hexane, cyclohexane, or petrol ether; aromatic hydrocarbons, preferably an aromatic C₆-C₁₀-hydrocarbons, such as benzene, toluene, o-, m-, and p-xylene; halogenated hydrocarbons, preferably halogenated aliphatic C₁-C₆-alkanes, or halogenated aromatic C₆-C₁₀-hydrocarbons, such as CH₂Cl₂, CHCl₃, CCl₄, CH₂ClCH₂Cl, CCl₃CH₃, CHCl₂CH₂Cl, CCl₂CCl₂, or chlorobenzene; ethers, preferably C₁-C₆-cycloalkyl ethers, C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, CH₃OCH₂CH₂OCH₃, dioxane, anisole, and THF; esters, preferably esters of aliphtic C₁-C₆-alcohols with aliphatic C₁-C₆-carboxylic acids, esters of aromatic C₆-C₁₀-alcohols with aromatic C₆-C₁₀-carboxylic adcids, cyclic esters of ω-hydroxy-C₁-C₆-carboxylic acids, such as CH₃C(O)OCH₂CH₃, CH₃C(O)OCH₃, CH₃C(O)OCH₂CH₂CH₂CH₃, CH₃C(O)OCH(CH₃)CH₂CH₃, CH₃C(O)OC(CH₃), CH₃CH₂CH₂C(O)OCH₂CH₃, CH₃CH(OH)C(O)OCH₂CH₃, CH₃CH(OH)C(O)OCH₃, CH₃C(O)OCH₂CH(CH₃)₂, CH₃C(O)OCH(CH₃)₂, CH₃CH₂C(O)OCH₃, benzyl benzoate, and γ-butyrolactone; carbonates, such as ethylene carbonate, propylene carbonate, CH₃CH₂OC(O)OCH₂CH₃, and CH₃OC(O)OCH₃; nitriles, preferably C₁-C₆-nitriles, such as CH₃CN, and CH₃CH₂CN; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and CH₃C(O)C(CH₃)₃ (MTBK); alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; amides and urea derivatives, preferably DMF, NMP, dimethyl acetamide (DMA), DMI, DMPU, HMPA; moreover DMSO, sulfolane, and water.

Suitable bases are, in general, inorganic bases, such as alkali metal and alkaline earth metal hydroxides, such as LiOH, NaOH, KOH, and Ca(OH)₂; alkali metal and alkaline earth metal ox-ides, such as Li₂O, Na₂O, CaO, and MgO; alkali metal and alkaline earth metal hydrides, such as LiH, NaH, KH and CaH₂; alkali metal and alkaline earth metal carbonates, such as Li₂CO₃, K₂CO₃ and CaCO₃; alkali metal bicarbonates, such as NaHCO₃; alkali metal phosphates, such as NaH₂PO₄, Na₂HPO₄, Na₃PO₄, KH₂PO₄, K₂HPO₄, K₄PO₄; organic bases, for example secondary amines, such as pyrrolidine; tertiary amines, such as diisopropylethylamine, trimethylamine, triethylamine, triisopropylamine and N-methylpiperidine, imidazol, pyridine; substituted pyridines, such as collidine, lutidine and 4-dimethylaminopyridine, and also polycyclic amides, such as DBU, DABCO; or NH₃. Mixtures of the aforementioned bases are also possible. The bases are generally employed in catalytic amounts; however, they can also be used in equimolar amounts, in excess or, if appropriate, as solvent.

Compounds of formula (16) may then be reacted with compounds of formula (10) to yield compounds of formula (11) under conditions as described for the reaction of compounds of formula (9) with compounds of formula (10) under General Scheme 1.

Compounds of formula (17) falling under the definition of formula (I), wherein M is S, may be prepared by a series of synthesis steps as displayed under General Scheme 3.

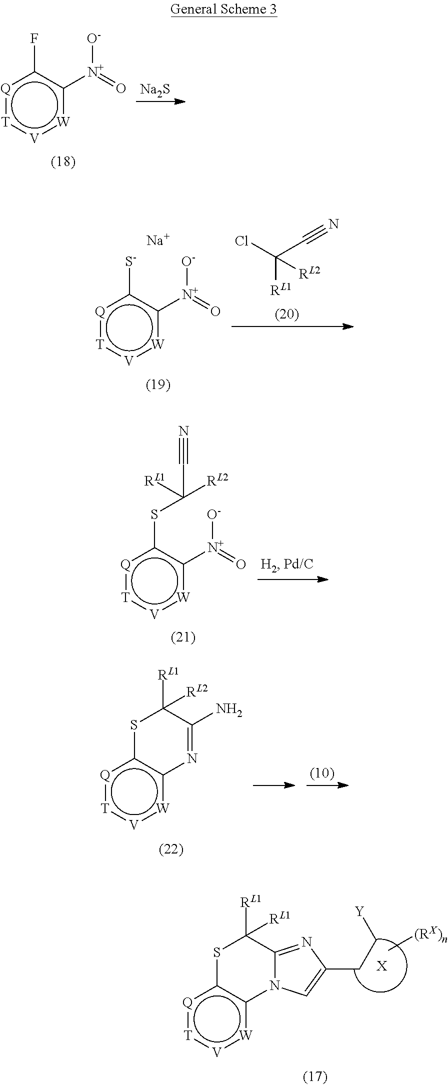

Compounds of formula (23) falling under the definition of formula (I), wherein L is S, may be prepared by a series of synthesis steps as displayed under General Scheme 4.

In a first step, compounds of formula (18) are reacted with N₂S in an inert solvent at a temperature of from −10 to 25° C. to afford compounds of formula (19). Suitable solvents include ethers, preferably C₁-C₆-cycloalkyl ethers, C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, CH₃OCH₂CH₂OCH₃, dioxane, anisole, and THF; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and CH₃C(O)C(CH₃)₃ (MTBK); alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; amides and urea derivatives, preferably DMF, NMP, dimethyl acetamide (DMA), DMI, DMPU, HMPA; moreover DMSO, sulfolane, water and mixtures thereof.

Compounds of formula (19) may then be reacted with compounds of formula (20) in an inert solvent at a temperature of from −10 to 40° C. to yield compounds of formula (21). Suitable solvents are aliphatic hydrocarbons, preferably an aliphatic C₅-C₁₆-hydrocarbon, more preferably a C₅-C₁₆-alkane, or C₅-C₁₆-cycloalkane, such as pentane, hexane, cyclohexane, or petrol ether; aromatic hydrocarbons, preferably an aromatic C₆-C₁₀-hydrocarbons, such as benzene, toluene, o-, m-, and p-xylene; halogenated hydrocarbons, preferably halogenated aliphatic C₁-C₆-alkanes, or halogenated aromatic C₆-C₁₀-hydrocarbons, such as CH₂Cl₂, CHCl₃, CCl₄, CH₂ClCH₂Cl, CCl₃OH₃, CHCl₂CH₂Cl, CCl₂CCl₂, or chlorobenzene; ethers, preferably C₁-C₆-cycloalkyl ethers, C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, CH₃OCH₂CH₂OCH₃, dioxane, anisole, and THF; alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; amides and urea derivatives, preferably DMF, NMP, dimethyl acetamide (DMA), DMI, DMPU, HMPA; moreover DMSO, sulfolane, and water.

Compouns of formula (21) may then be reduced by reaction with H₂ to yield compounds of formula (22) under reaction conditions as described for the reaction of compounds of formula (5) to compounds of formula (6) under General Scheme 1 above.

Compounds of formula (22) may then be reacted with compounds of formula (10) to yield compounds of formula (17) under reaction conditions as described for the reaction of compounds of formula (9) with compounds of formula (10) under General Scheme 1 above.

Compounds of formula (23) falling under the definition of formula (I), wherein L is S, may be prepared by a series of synthesis steps as displayed under General Scheme 4.

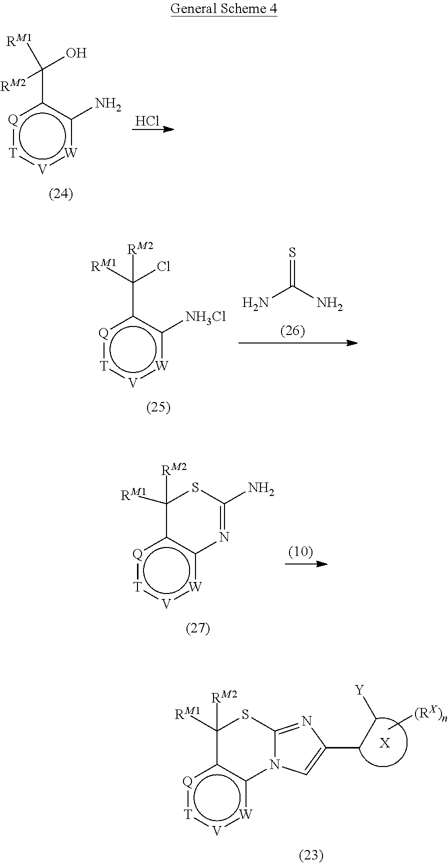

In a first step, compounds of formula (24) may be reacted with HCl in a polar solvent such as H₂O to yield compounds of formula (25). The reaction is typically carried out at a temperature of from 80 to 140° C.

Then, compounds of formula (25) may be reacted with thiourea (26) to yield compounds of formula (27). The reaction is typically carried out at elevated temperatures of from 50 to 120° C. in an inert solvent. Typical solvents are polar organic solvents. Suitable solvents includes ethers, preferably C₁-C₆-cycloalkyl ethers, C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, CH₃OCH₂CH₂OCH₃, dioxane, anisole, and THF; alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH, and mixtures thereof.

Compounds of formula (27) may then be reacted with compounds of formula (10) to yield compounds of formula (23). The reaction may be carried out under reaction conditions as described for the reaction of compounds of formula (9) with compounds of formula (10) under General Scheme 1 above.

Compounds of formula (28), falling under the definition of compounds of formula (I), wherein A is N and E is NR^{E} may for example be prepared as described under General Scheme 5.

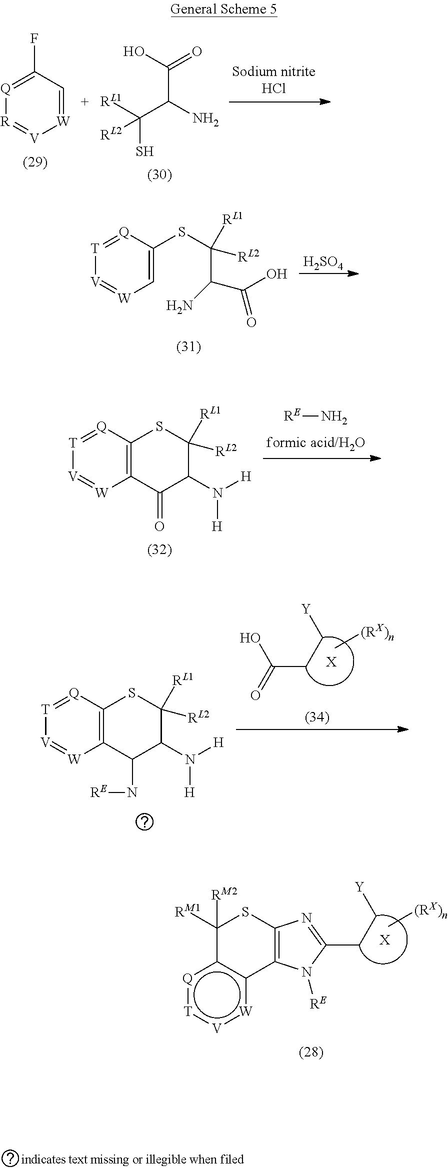

Compounds of formulae (29) and (30) are commercially available. They may be reacted in the presence of HCl and sodium nitrate under reaction conditions as described for example in Patt, J. T.; et al Journal of Labelled Compounds & Radiopharmaceuticals (2002), 45(14), 1229-1238. Compounds of formula (31) may then be converted to compounds of formula (32) in the presence of an acid like H2SO4 as described in Meng, Ling; et al Organic Letters (2020), 22(3), 1155-1159. In a next step, compounds of formula (32) may be reacted with amine compounds R^{E}—NH₂ in the presence of formic acid to yield compounds of formula (33). Such reactions have been described in Sindelar, K.; et al Collection of Czechoslovak Chemical Communications (1968), 33(12), 4315-27.

Finally, compounds of formula (33) may be reacted with compounds of formula (34) to yield compounds of formula (28). Such reactions have been previously described for example in EP application number 20168197.0, WO2016162318A1, p. 90, and EP3257853A1, e.g. p. 33-34.

By other way of example, compounds of formula (35), falling under the definition of compounds of formula (I), wherein A is N and E is NR^{E}, may be prepared as described under General Scheme 6.

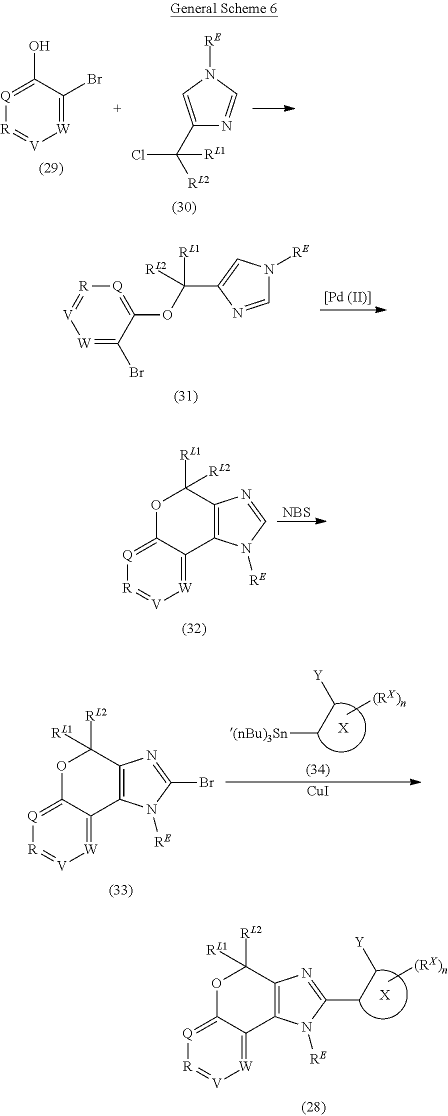

NBS is N-bromosuccinimmide; nBu is n-butyl

Compounds of formula (29) and (30) are commercially available or can be prepared by standard methods of organic chemistry. In a first step, compounds of formula (29) and compounds of formula (30) may be reacted to obtain compound (31). Typically, the reaction is carried out in a polar organic solvent, e.g. DMF, in the presence of a catalys, such as a metal salt, preferably a Cs-salt, at a temperature of from 10 to 50° C.

Compounds of formula (31) may then be converted to compounds (32) by catalysis with a Pd(II)-salt and a base in a Heck-type reaction. The reaction is typically carried out at a temperature of from 10 to 120° C. in a polar aprotic solvent, such a DMSO. Typical bases include those as described above under General Scheme 1, preferably alkali salts of acetic acid, such as potassium acetate or sodium acetate, alkali carbonates, such as potassium carbonate, and organic amine bases such as triethylamine. Typical Pd(II)-salts include halogenides and organic anions of Pd(II), such as acetate, and fumarate. Usually, a ligand is also added to the composition such as triphenylphosphan, phosphinooxazolines, or (2,2′-bis(diphenylphosphino)-1,1′-binaphthyl).

Compounds (32) may then be brominated to afford compounds (33). Typical brominating agents include N-bromosuccinimmide (NBS). Such reactions are generally carried out at a temperature of from 10 to 50° C. in a polar, aprotic solvent, such as DMF. Usually, an excess of NBS is applied. Typical ratios of compounds (32) to NBS may be from 1:1 to 1:10.

Finally, compounds (33) may be coupled with compounds (34) to obtain compounds (28) in a Stille-type reaction. Compounds (34) are available by standard methods from the respective bromine analogues of compounds (34). Such reactions include the use of a Pd(0)-additive, which may be generated in-situ by using a Pd(II)-salt. Typicaly further additives include Cu(I)-salts, in particular CuI. The reaction is typically carried out in an apolar organic solvent, such as a hydrocarbon solvent, in particular an aromatic hydrocarbon solvent, such as benzene or toluene. The reaction may be carried out at a temperature of from 50 to 150° C.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

The N-oxides may be prepared from the inventive compounds according to conventional oxidation methods, e. g. by treating compounds of formula (I) with an organic peracid such as metachloroperbenzoic acid (cf. WO 03/64572 or J. Med. Chem. 38(11), 1892-903, 1995); or with inorganic oxidizing agents such as hydrogen peroxide (cf. J. Heterocyc. Chem. 18(7), 1305-8, 1981) or oxone (cf. J. Am. Chem. Soc. 123(25), 5962-5973, 2001). The oxidation may lead to pure mono-N-oxides or to a mixture of different N-oxides, which can be separated by conventional methods such as chromatography.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

A skilled person will readily understand that the preferences for the substituents, also in particular the ones given in the tables below for the respective substituents, given herein in connection with compounds of formula (I) apply for the intermediates accordingly. Thereby, the substituents in each case have independently of each other or more preferably in combination the meanings as defined herein.

### Preferences

Embodiments and preferred compounds of the present invention for use in pesticidal methods and for insecticidal application purposes are outlined in the following paragraphs. The remarks made below concerning preferred embodiments of the variables of compounds of formula (I) are valid both on their own in combination with each other. The variables of the compounds of formula (I) have the following meanings, these meanings, both on their own and in combination with one another, being particular embodiments of the compounds of the formula (I). The following preferences are also valid for compounds of formula (1) to formula (33) above.

The variable A is CH, N, or NH. In one embodiment, A is N. In another embodiment, A is NH. The variable E is N, NH, O, S, or CR^{E}. In one embodiment, E is NR^{E} or CR^{E}. In another embodiment, A is N or NH, and E is NR^{E} or CR^{E}. In another embodiment, A is N and E is CR^{E}.

The variables G and J are independently C or N, provided that only one of E or G is N. Typically, both G and J are C. In one embodiment, G is N and J is C.

The variable Q is N or CRQ. In one embodiment, the variable Q is N. In another embodiment, the variable Q is CR^{Q}, preferably wherein R^{Q} is H, C₁-C₃-alkyl, C₁-C₃-haloalkyl, C₁-C₃-haloalkoxy, or C₁-C₃-alkoxy, more preferably wherein R^{Q} is H, C₁-C₃-fluoroalkyl, or C₁-C₃-fluoroalkoxy, most preferably wherein R^{Q} is H, CF₃, OCHF₂, or OCF₃, especially preferably wherein R^{Q} is H, CF₃, or OCF₃, such as H or CF_{3 .} In one embodiment, the variable Q is CRQ, preferably wherein R^{Q} is C₁-C₃-alkyl, C₁-C₃-haloalkyl, C₁-C₃-haloalkoxy, or C₁-C₃-alkoxy, more preferably wherein R^{Q} is C₁-C₃-alkoxy, C₁-C₃-fluoroalkyl, or C₁-C₃-fluoroalkoxy, most preferably wherein R^{Q} is CF₃, OCHF₂, or OCF₃, especially preferably wherein R^{Q} is CF₃, or OCF₃.

The variable T is N or C^{T}. In one embodiment, the variable Q is N. In another embodiment, the variable T is CR^{T}, preferably wherein R^{T} is H, C₁-C₃-alkyl, C₁-C₃-haloalkyl, C₁-C₃-alkoxy, or C₁-C₃-haloalkoxy, more preferably wherein R^{T} is H, C₁-C₃-fluoroalkyl, or C₁-C₃-fluoroalkoxy, most preferably wherein R^{T} is H, or CF₃, such as H.

The variable V is N or CR^{V}. In one embodiment, the variable V is N. In another embodiment, the variable V is CR^{V}, preferably wherein R^{V} is H, C₁-C₃-alkyl, C₁-C₃-haloalkyl, C₁-C₃-alkoxy, or C₁-C₃-haloalkoxy, more preferably wherein R^{V} is H, C₁-C₃-fluoroalkyl, or C₁-C₃-fluoroalkoxy, most preferably wherein R^{V} is H, CF₃ or OCF₃, especially preferably wherein R^{V} is H or CF₃, in particular wherein R^{V} is CF₃.

The variable W is N or CR^{W}. In one embodiment, the variable W is N. In another embodiment, the variable W is CR^{W}, preferably wherein R^{W} is H, C₁-C₃-alkyl, C₁-C₃-haloalkyl, C₁-C₃-alkoxy or C₁-C₃-haloalkoxy, more preferably wherein R^{W} is H, C₁-C₃-fluoroalkyl, or C₁-C₃-fluoroalkoxy, most preferably wherein R^{W} is H, CF₃ or OCF₃, especially preferably wherein R^{W} is H.

The group -M-L- is selected from O—CR^{L1}R^{L2}, S—CR^{L1}R^{L2}, CR^{M1}R^{M2}—O, or CR^{M1}R^{M2}—S. In other words, L may be O or S as long as M is CR^{M1}R^{M2}; and M may be O or S as long as L is CR^{L1}R^{L2}. In one embodiment, M is O and L is CR^{L1}R^{L2}. In another embodiment, M is S and L is CR^{L1}R^{L2}. In another embodiment, L is O and M is CR^{M1}R^{M2}. In another embodiment, L is S and M is CR^{M1}R^{M2}.

Prefered combinations of variables A, E, G, J, Q, T, V, and W are presented below as formulae (I-A) to (I-BK), wherein the variables have a meaning as defiend for formula (I).

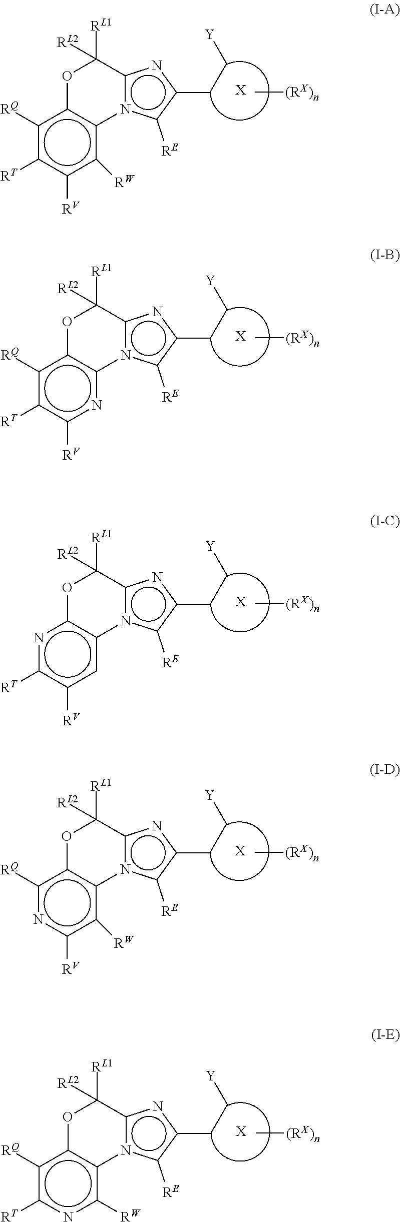

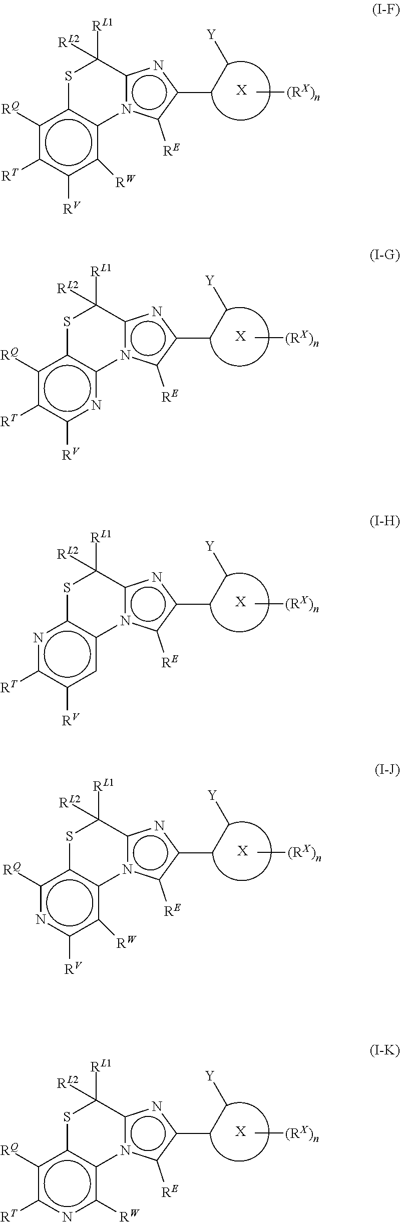

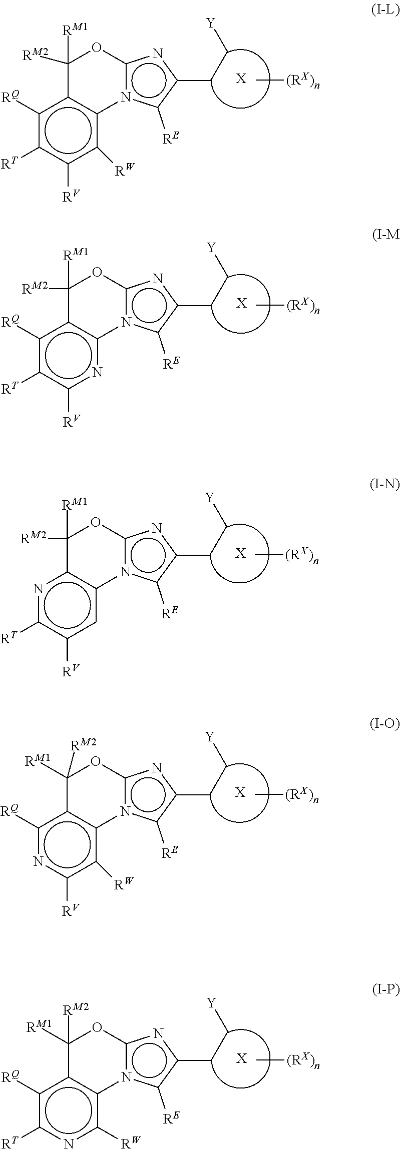

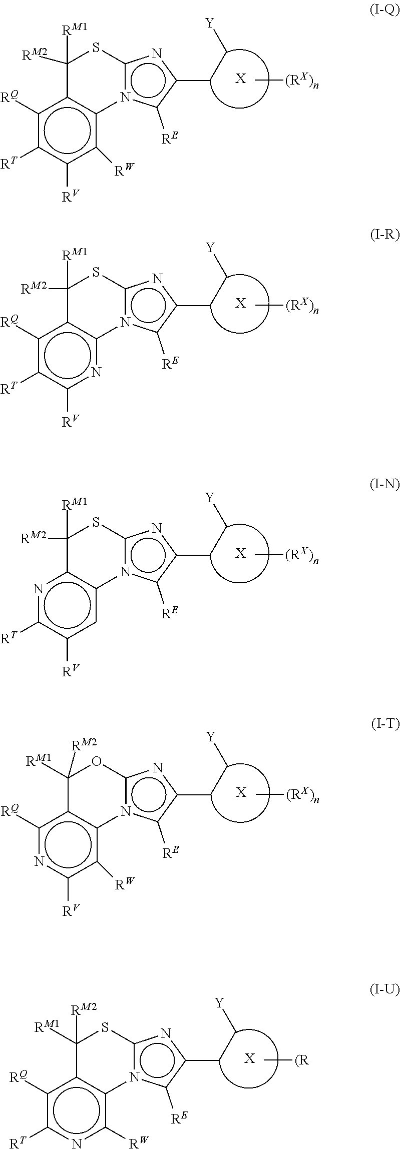

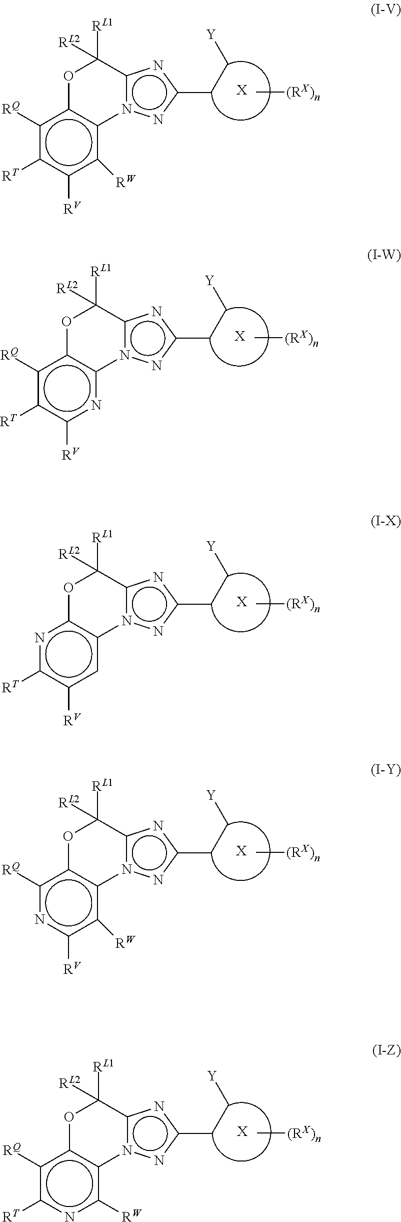

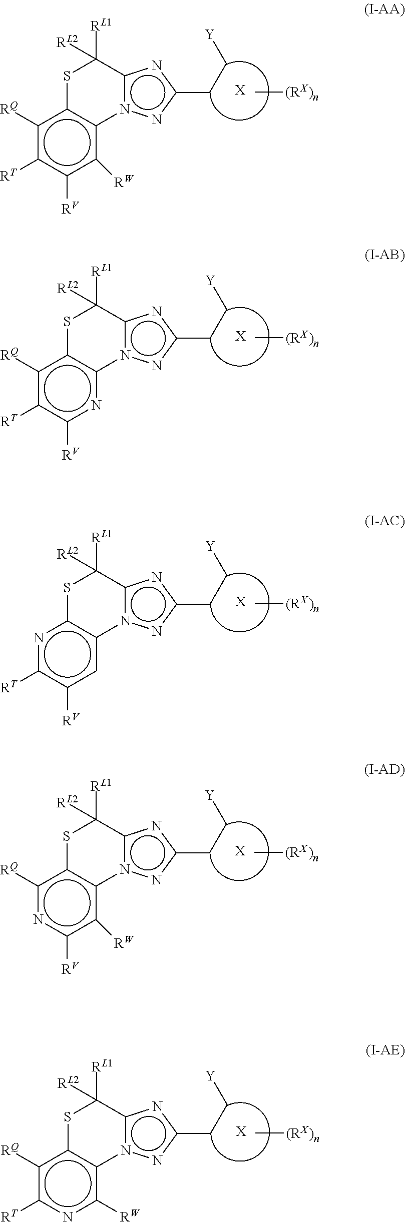

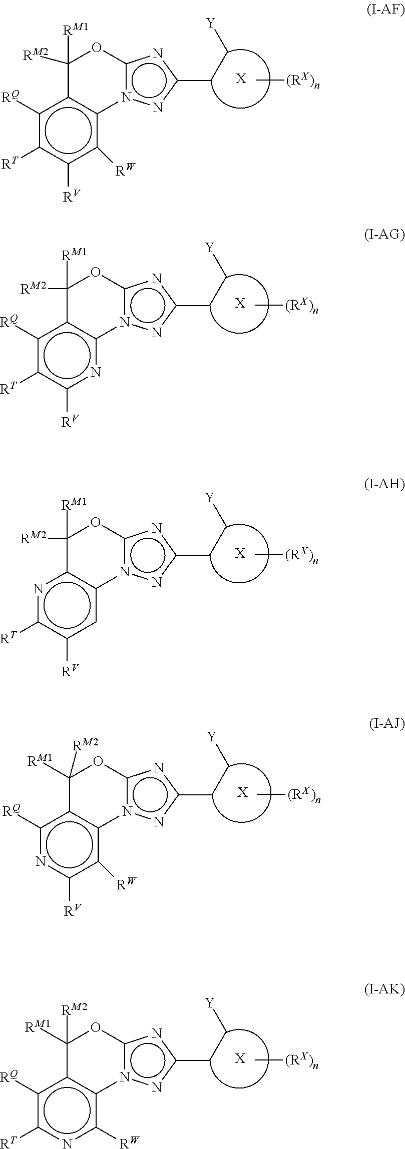

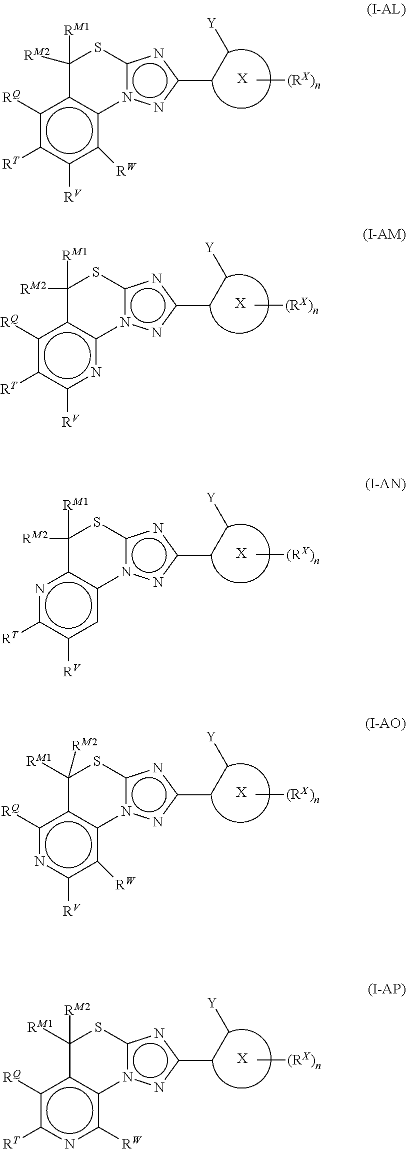

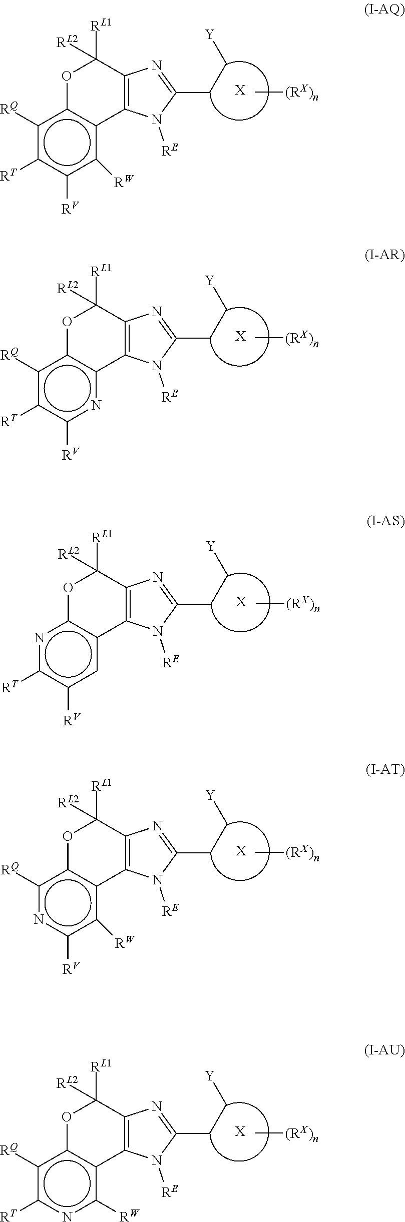

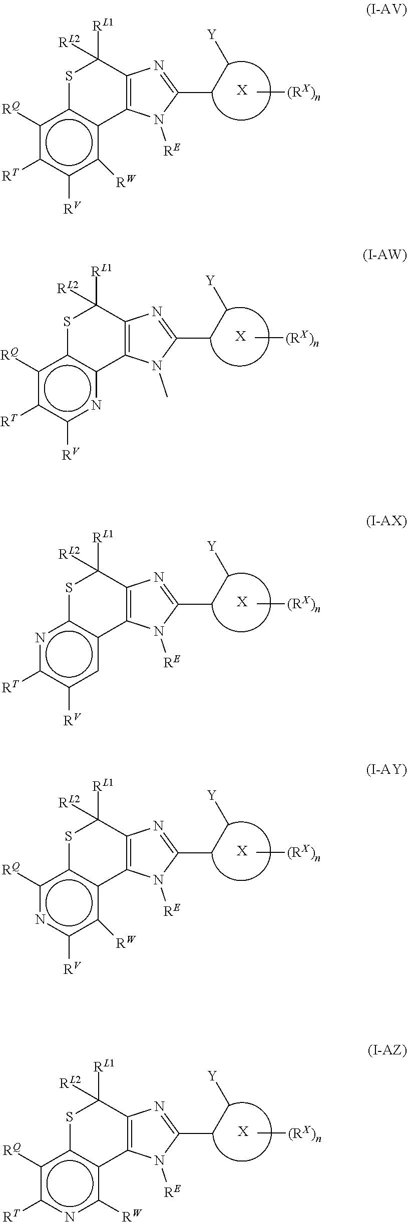

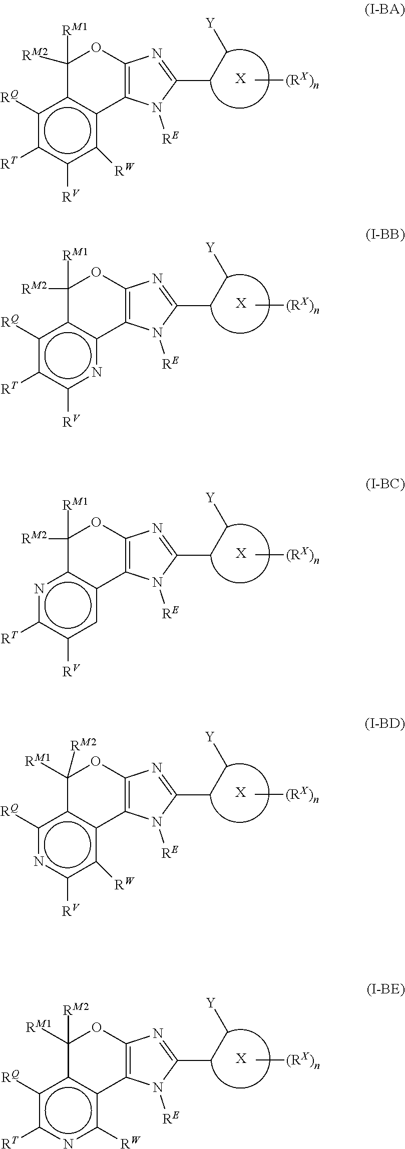

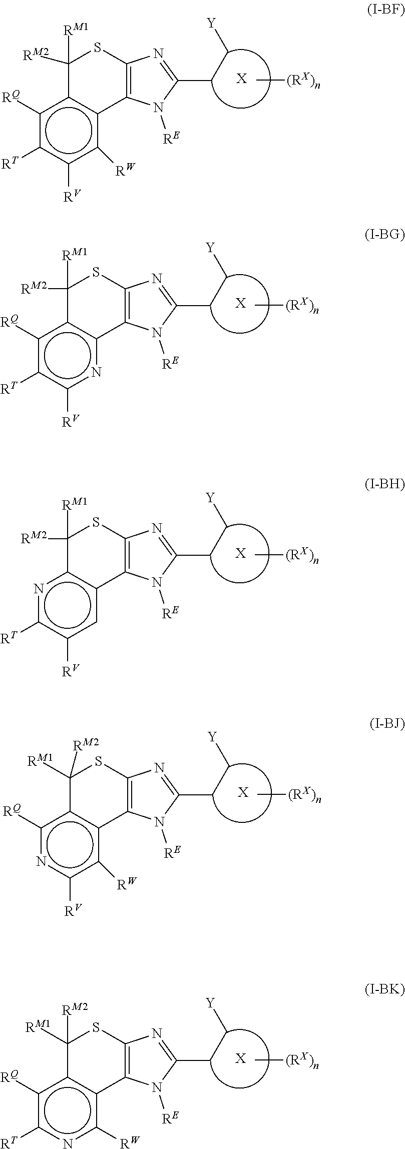

In one embodiment, compounds of formula (I) are compounds of formula (I-A), (I-F), (I-L), (I-Q), (I-B), (I-M), (I-R), (I-G), (I-AQ), (I-AV), (I-BA), or (I-BF). In another embodiment, compounds of formula (I) are compounds of formula (I-A), (I-F), (I-L), (I-Q), (I-B), (I-M), (I-R), or (I-G). In another embodiment, compounds of formula (I) are compounds of formula (I-A), (I-F), (I-L), (I-Q), (I-AQ), (I-AV), (I-BA), or (I-BF).

Typically, at least one of the variables Q, T, V or W is not N. In another embodiment, all varia-bles Q, T, V, and W are not N.

Accordingly, compounds of formula (I) are preferably of formulae (II), (III) or (IV)

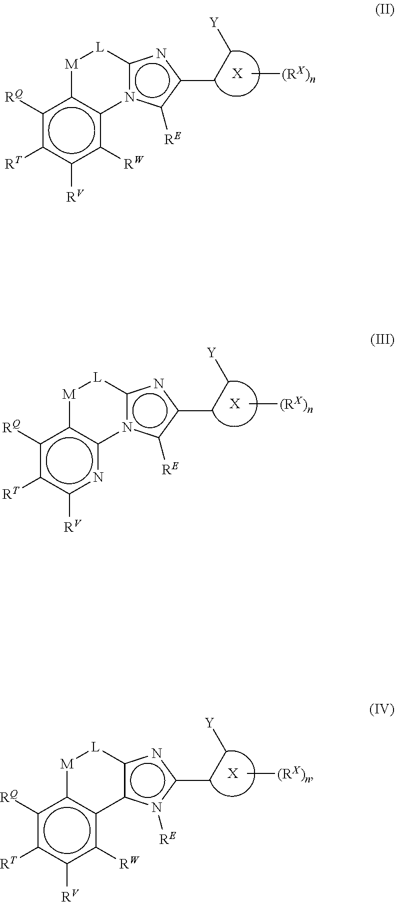

wherein the variables have a meaning as defined for formula (I). In one embodiment, compounds of formula (I) are compounds of formula (II). In another embodiment, compounds of formula (I) are compounds of formula (III). In another embodiment, compounds of formula (I) are compounds of formula (IV). In another embodiment, compounds of formula (I) are are compounds of formula (II) or formula (IV).

The variable Y is SR^{Y1}, S(O)R^{Y1}, S(O)₂R^{Y1}, S(═O)(═NR^{Y2})R^{Y1}, or S(═NR^{Y2})(═NR^{Y3})R^{Y1}. In one embodiment, the variable Y is SR^{Y1}. In another embodiment, the variable Y is S(O)R^{Y1}. In another embodiment, the variable Y is S(O)₂R^{Y1}. In another embodiment, the variable Y is S(═O)(═NR^{Y2})R^{Y1}. In another embodiment, the variable Y is S(═NR^{Y2})(═NR^{Y3})R^{Y1}.

Each R^{Y1}, R^{Y2}, R^{Y3} is independently selected from H, C₁-C₆-alkyl, C₃-C₆-cycloalkyl, C₂-C₆-alkenyl, C₃-C₆-cycloalkenyl, C₂-C₆-alkynyl, C₃-C₆-cycloalkyl-C₁-C₃-alkyl, which groups are unsubstituted, or substituted with one or more, same or different substituents selected from halogen and CN;
a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents selected from halogen, CN, C₁-C₃-alkyl, C₁-C₃-alkoxy, C₁-C₃-haloalkoxy, and wherein said N- and S-atoms are independently oxidized, or non-oxidized;
phenyl, which is unsubstituted, or substituted with one or more, same or different substituents selected from halogen, CN, C₁-C₃-alkyl, C₁-C₃-alkoxy, C₁-C₃-haloalkoxy;
or two substituents selected from R^{Y1}, R^{Y2}, R^{Y3} form, together with the S- or N-atoms to which they are bound, a 5- or 6- membered saturated, partially unsaturated, or fully unsaturated heterocycle, which heterocycle is unsubstituted, or substituted with one or more, same or different substituents selected from halogen, CN, C₁-C₃-alkyl, C₁-C₃-alkoxy, C₁-C₃-haloalkyl, C₁-C₃-haloalkox, and wherein said heterocycle comprises no, one or more, same or different heteroatoms O, N, or S in addition to the N- or S-atoms to which R^{Y1} and R^{Y2} are bound to.

In another embodiment, each R^{Y1}, R^{Y2}, R^{Y3} is independently selected from H, C₁-C₃-alkyl, which is unsubstituted, or substituted with one or more, same or different substituents selected from halogen; phenyl, which is unsubstituted, or substituted with one or more, same or different substituents selected from halogen, CN, C₁-C₃-alkyl, and C₁-C₃-haloalkyl; or two substituents selected from R^{Y1}, R^{Y2}, R^{Y3} form, together with the heteroatoms to which they are bound, a 5- or 6- membered partially unsaturated, or fully unsaturated heterocycle, which heterocycle is unsubstituted, or substituted with halogen, and wherein said heterocycle comprises no, one or more, same or different heteroatoms O, N, or S in addition to the heteroatoms to the two substituents selected from R^{Y1}, R^{Y2}, and R^{Y3} are bound to.

Typically, each R^{Y1}, R^{Y2}, R^{Y3} is selected from H, C₁-C₃-alkyl and C₁-C₃-haloalkyl. Preferably, R^{Y1} is selected from C₁-C₃-alkyl and C₁-C₃-haloalkyl, and R^{Y2} is selected from H, C₁-C₃-alkyl, and C₁-C₃-haloalkyl.

Accordingly, Y is preferably SO₂R^{Y1}, or S(═O)(═NR^{Y2})R^{Y1}, wherein R^{Y1} is C₁-C₃-alkyl or C₁-C₃-haloalkyl, and R^{Y2} is H, C₁-C₃-alkyl, or C₁-C₃-haloalkyl. More preferably, Y is SO₂R^{Y1}.

In another embodiment, R^{Y1} is C₁-C₆-alkyl, C₃-C₆-cycloalkyl, C₃-C₆-cycloalkyl-C₁-C₄-alkyl, which groups are halogenated or non-halogenated; CH₂R⁶, or phenyl, which is unsubstituted or substituted with R¹¹. Typically, R^{Y1} is C₁-C₄-alkyl, which is halogenated or non-halogenated, preferably C₁-C₃-alkyl, or C₁-C₃-haloalkyl, preferably CH₃CH₂.

The index n is 0, 1, 2, 3, or 4, if X is phenyl, or a 6-membered heteroaryl, or 0, 1, 2, or 3 if X is a 5-membered heteroaryl, preferably phenyl or 2-pyridyl Typically, n is 1. In one embodiment, n is 0. In another embodiment, n is 2. In another embodiment, n is 3.

R^{E}, R^{Q}, R^{T}, R^{V}, and R^{W} are independently H, halogen, N₃, CN, NO₂, SCN, SF₅; C₁-C₆-alkyl, C₁-C₆-alkoxy, C₂-C₆-alkenyl, tri-C₁-C₆-alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxyx-C₁-C₄-alkyl, which groups are halogenated or non-halogenated, C(═O)OR¹, NR²R³, C(═O)NR²R³, C(═O)R⁴, SO₂NR²R³, S(═O)ₘR⁵, OR⁶, SR⁶, or CH₂R⁶; phenyl, which is unsubstituted or substituted with one or more, same or different substituents R¹¹.

R^{E} is typically H, halogen; C₁-C₃-alkyl, C₁-C₃-alkoxy, C₂-C₃-alkenyl, C₂-C₃-alkynyl, C₃-C₅-cycloalkyl, which groups are halogenated or non-halogenated. In one embodiment, R^{E} is H, C₁-C₃-alkyl, or C₁-C₃-haloalkyl. In another embodiment, R^{E} is H, C₁-C₃-alkyl, C₁-C₃-alkoxy, which groups are unsubstituted or halogenate. In another embodiment, R^{E} is H or CH₃. In another embodiment, R^{E} is CH₃. In another embodiment, R^{E} is H.

R^{Q} is typically H, halogen; C₁-C₃-alkyl, C₁-C₃-alkoxy, C₂-C₃-alkenyl, C₂-C₃-alkynyl, C₃-C₅-cycloalkyl, which groups are halogenated or non-halogenated. In one embodiment, R^{Q} is H, C₁-C₃-alkyl, C₁-C₃-haloalkyl, C₁-C₃-alkoxy, or C₁-C₃-haloalkoxy. In another embodiment, R^{Q} is H, CHF₂, CF₃, OCHF₂, or OCF₃. In another embodiment, R^{Q} is H, CF₃ or OCF₃. In another embodiment, R^{Q} is H or CF₃. In one embodiment, R^{Q} is C₁-C₃-haloalkyl, C₁-C₃-alkoxy, or C₁-C₃-haloalkoxy, preferably C₁-C₃-haloalkyl, or C₁-C₃-haloalkoxy. In another embodiment, R^{Q} is CF₃, or OCF₃.

R^{T} is typically H, halogen; C₁-C₃-alkyl, C₁-C₃-alkoxy, C₂-C₃-alkenyl, C₂-C₃-alkynyl, C₃-C₅-cycloalkyl, which groups are halogenated or non-halogenated. In one embodiment, R^{T} is H, C₁-C₃-alkyl, C₁-C₃-haloalkyl, C₁-C₃-alkoxy, or C₁-C₃-haloalkoxy. In another embodiment, R^{T} is H, CHF₂, CF₃, OCHF₂, or OCF₃. In another embodiment, R^{Q} is R^{T} is H, C₁-C₃-haloalkyl, or C₁-C₃-haloalkoxy. In another embodiment, R^{T} is H, or CF₃. In another embodiment, R^{T} is H.

R^{V} is typically H, halogen; C₁-C₃-alkyl, C₁-C₃-alkoxy, C₂-C₃-alkenyl, C₂-C₃-alkynyl, C₃-C₅-cycloalkyl, which groups are halogenated or non-halogenated. In one embodiment, R^{V} is H, C₁-C₃-haloalkyl, C₁-C₃-alkoxy, or C₁-C₃-haloalkoxy. In another embodiment, R^{V} is H, CHF₂, CF₃, OCHF₂, or OCF₃. In another embodiment, R^{V} is H, CF₃ or OCF₃. In another embodinvent, R^{V} is H or CF₃. In another embodiment, R^{V} is H.

R^{W} is typically H, halogen; C₁-C₃-alkyl, C₁-C₃-alkoxy, C₂-C₃-alkenyl, C₂-C₃-alkynyl, C₃-C₅-cycloalkyl, which groups are halogenated or non-halogenated. In one embodiment, R^{V} is H, C₁-C₃-alkyl, C₁-C₃-haloalkyl, C₁-C₃-alkoxy, or C₁-C₃-haloalkoxy. In another embodiment, R^{W} is H, CHF₂, CF₃, OCHF₂, or OCF₃. In another embodiment, R^{W} is H, CF₃ or OCF₃. In another embodiment, R^{W} is H or CF₃. In another embodiment, R^{W} is H.

Accordingly, R^{Q}, R^{T}, R^{V} and R^{W} are typically independently H; or C₁-C₃-alkyl, C₁-C₃-alkoxy, which groups are halogenated or non-halogenated.

The cycle X is phenyl, or a 5- or 6-membered heteroaryl, preferably 2-pyridyl. For the avoidance of doubt, the cycle X is substituted with n substituents R^{X}. Also, for the avoidance of doubt, X is connected to Y and to the tricyclic system by direct chemical bonds to two adjacent ring members of X. Accordingly, compounds of formula (I) do not comprise any compounds wherein X is not connected to Y and to the tricyclic system by direct chemical bonds to two adjacent ring members of X, such as in 4-(8-fluoro-4H-thieno[3,2-c][1]benzopyran-2-yl)-2-(methylsulfonyl)-pyrimidine (CAS1099595-55-7).

In one embodiment, X is phenyl. In another embodiment, X is a 5-membered heteroaryl. In another embodiment, X is a 6-membered heteroaryl. In another embodiment, X is a 5-membered heteroaryl comprising one N-atom. In another embodiment, X is a 6-membered heteroaryl comprising at least one N-atom. In another embodiment, X is a 6-membered heteroaryl comprising two N-atoms.

Prefered 5- or 6-membered heteroaryls X are depicted below as formulae A-1 to A-48, wherein “&” stands for the connection to the trycyclic scaffold of compounds of formula (I). For the avoidance of doubt, the formulae A-1 to A-48 are preferred embodiments on their own and in combination for the following moiety of formula

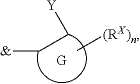

wherein “&” stands for the connection to the tricyclic scaffold in formula (I). In other words, the substituents Y and (R^{X})ₙ in formulae A-1 to A-48 are mere illustrations but are not part of the heteroaryl X.

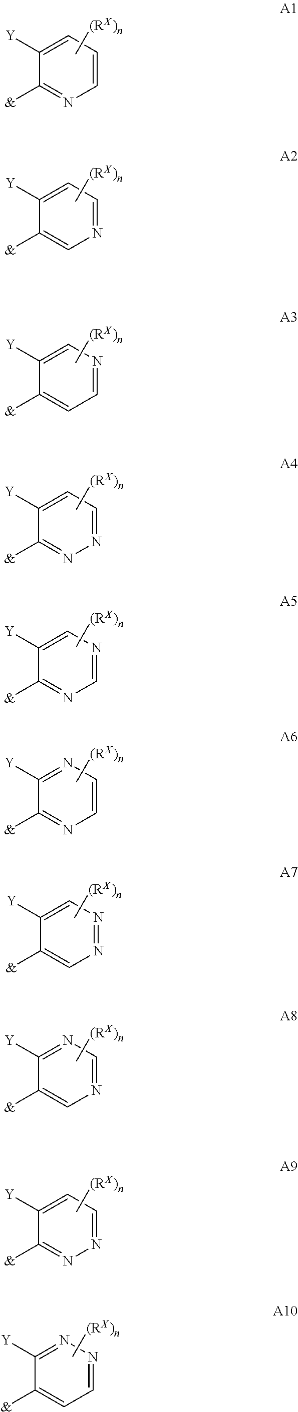

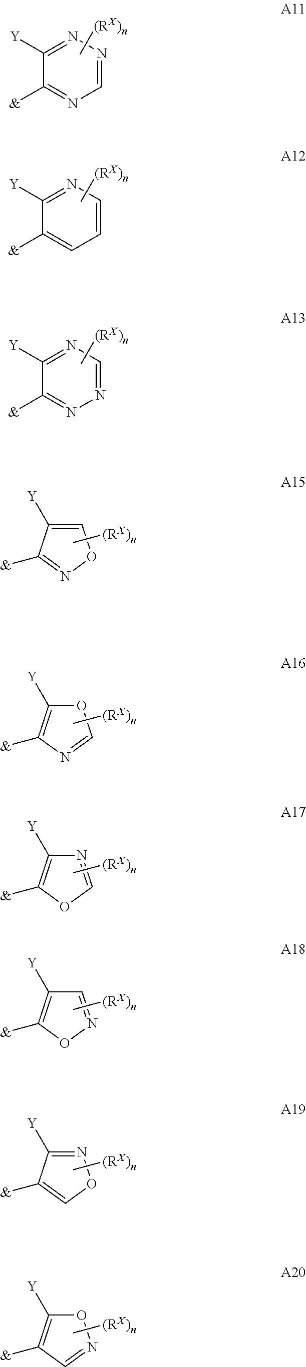

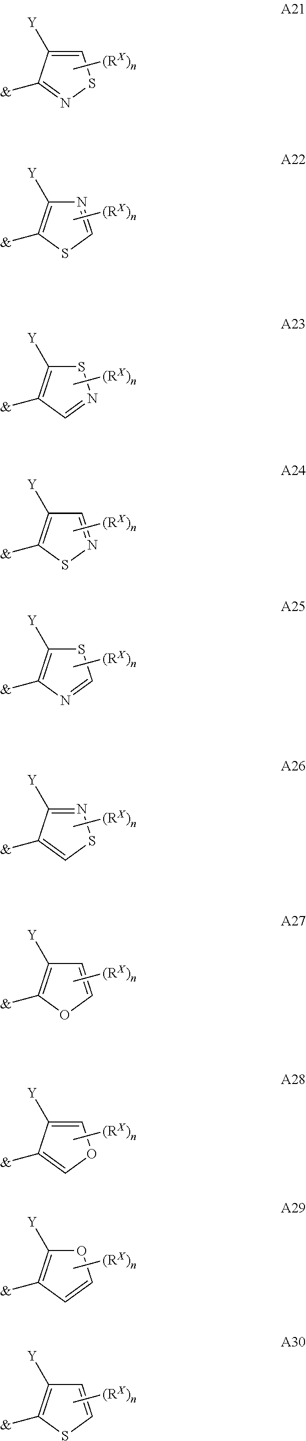

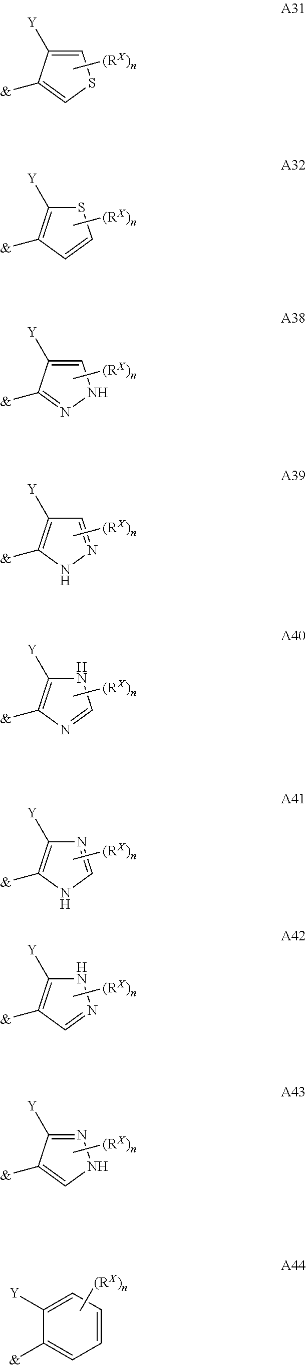

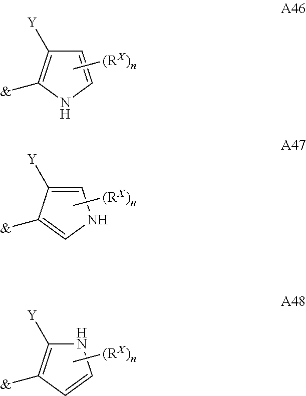

In one embodiment, X is selected from formulae A-1 to A-14. In one embodiment, X is selected from formulae A-1 to A-3. In another embodiment, Xis A-1. In another embodiment, X is A-2. In another embodiment, X is A-3.

R¹ is H; C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆-cycloalkoxy-C₁-C₄-alkyl, which groups are halogenated or non-halogenated; C₁-C₆-alkylen-NR²R³, C₁-C₆-alkylen-CN, or CH₂R⁶; or phenyl, which is unsubstituted, or substituted with one or more, same or different substituents R¹¹.

In one embodiment, R¹ is phenyl, which is unsubstituted or substituted with one or more, same or different substituents R¹¹. In another embodiment, R¹ is H; C₁-C₆-alkyl, C₂-C₆-alkenyl, C₂-C₆-alkynyl, C₁-C₆-alkoxy-C₁-C₄-alkyl, C₃-C₆-cycloalkyl, C₃-Calkyl-C₁-C₄-alkyl, or C₃-C₆-cycloalkoxy-C₁-C₄-alkyl, which groups are halogenated or non-halogenated. In another embodiment, R¹ is H; C₁-C₃-alkyl, C₂-C₃-alkenyl, C₂-C₃-alkynyl, which groups are halogenated or non-halogenated. In another embodiment, R¹ is C₁-C₃-alkyl or C₁-C₃-haloalkyl.

R¹¹ is halogen, N₃, OH, CN, NO₂, SCN, SF₅; C₁-C₆-alkyl, C₁-C₆-alkoxy, C₂-C₆-alkenyl, C₂-C₆-alkynyl, C₁-C₆-alkoxy-C₁-C₄-alkoxy, C₁-C₆-alkoxy-C₁-C₄-alkyl, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, which groups are halogenated or non-halogenated.

In one embodiment, R¹¹ is halogen, OH, CN, SF₅; C₁-C₆-alkyl, C₁-C₃-alkoxy, which groups are halogenated or non-halogenated. In one embodiment, R¹¹ is halogen; C₁-C₃-alkyl, or C₁-C₃-haloalkyl.

R² is H; C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are halogenated or non-halogenated; C(═O)R²¹, C(═O)OR²¹, C(═O)NR²¹, C₁-C₆-alkylen-CN, or CH₂R⁶; or phenyl, which is unsubstituted or substituted with one or more, same or different substituents R¹¹. In one embodiment, R² is H; C₁-C₆-alkyl, C₂-C₃-alkenyl, C₂-C₃-alkynyl, which groups are halogenated or non-halogenated. In another embodiment, R² is H. In another embodiment, R² is H; C₁-C₃-alkyl, or C₁-C₃-haloalkyl.

R²¹ is H; C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl; C₃-C₆-cycloalkyl-C₁-C₄-alkyl, C₃-C₆-cycloalkoxy-C₁-C₄ alkyl, phenyl, or a saturated, partially-, or fully unsaturated 5- or 6-membered heterocycle, wherein the cyclic moieties are unsubstituted or substituted with one or more, same or different substituents R¹¹. In one embodiment, R²¹ is H; C₁-C₃-alkyl, C₁-C₃-haloalkyl, or phenyl. In another embodiment, R²¹ is C₁-C₃-alkyl.

R³ is H; C₁-C₆-alkyl, C₂-C₆-alkenyl, C₂-C₆-alkynyl, C₁-C₆-cycloalkyl-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are halogenated or non-halogenated; C₁-C₆-alkylen-CN, or CH₂R⁶; phenyl, which is unsubstituted or substituted with one or more, same or different substituents R¹¹; or NR²R³ may also form an N-bound, saturated 3- to 8-membered heterocycle, which in addition to the nitrogen atom may have 1 or 2 further heteroatoms or heteroatom moieties selected from O, S(═O)ₘ, NH, and N—C₁-C₆-alkyl, and wherein the N-bound heterocycle is unsubstituted or substituted with one or more, same or different substituents selected from halogen, C₁-C₄-alkyl, C₁-C₄-haloalkyl, C₁-C₄-alkoxy and C₁-C₄-haloalkoxy. In one embodiment, R³ is H, C₁-C₃-alkyl, C₁-C₃-haloalkyl, or phenyl. In another embodiment, R³ is phenyl. In another embodiment, R³ is H. In another embodiment, R² is H and R³ is C₁-C₃-alkyl or phenyl.

R⁴ is selected from H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which are unsubstituted or substituted with halogen; CH₂R⁶, or phenyl, which is unsubstituted, or substituted with one or more, same or different substituents R¹¹. In one embodiment, R⁴ is H, C₁-C₃-alkyl, C₁-C₃-haloalkyl, or phenyl. In another embodiment, R⁴ is H. In another embodiment, R⁴ is C₁-C₃-alkyl, or C₁-C₃-haloalkyl.

R⁵ C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆-cycloalkoxy-C₁-C₄-alkyl, which groups are halogenated or non-halogenated; C₁-C₆-alkylen-NR²R³, C₁-C₆-alkylen-CN, CH₂R⁶; or phenyl, which is unsubstituted, or substituted with one or more, same or different substituents R¹¹. In one embodiment, R⁵ is C₁-C₆-alkyl, C₂-C₆-alkenyl, C₂-C₆-alkynyl, or phenyl, which groups are unhalogenated or halogenated. In another embodiment, R⁵ is C₁-C₃-alkyl or C₁-C₃-haloalkyl.

R⁶ is phenyl, which is unsubstituted or substituted with one or more, same or different substituents R¹¹. In one embodiment, R⁶ is phenyl. In another embodiment, R⁶ is phenyl that is unsubsituted or substituted with halogen, C₁-C₃-alkyl, C₁-C₃-haloalkyl, C₁-C₃-alkoxy, or C₁-C₃-haloalkoxy.

Each R^{L1}, R^{L2}, R^{M1}, R^{M2} is independently H, halogen, OH; C₁-C₄-alkyl, C₃-C₆-cycloalkyl, C₃-C₆-cycloalkyl-C₁-C₂-alkyl, C₂-C₄-alkenyl, C₃-C₆-cycloalkenyl, C₂-C₄-alkynyl, C₁-C₄-alkoxy, which groups are unsubstituted, or substituted with one or more, same or different substituents selected from halogen and CN; phenyl, which is unsubstituted, or substituted with one or more, same or different substituents selected from halogen, CN, C₁-C₃-alkoxy, and C₁-C₃-haloalkoxy; or R^{L1} and R^{L2}, or R^{M1} and R^{M2} form together with the carbon atom they are bonded to a group C═O, C═S, C═NR⁷R⁸, or C═NOR⁷.

Typically, each R^{L1}, R^{L2}, R^{M1}, R^{M2} is independently H, C₁-C₃-alkyl, which groups are unsubstituted, or substituted with one or more, same or different substituents selected from halogen and CN; or R^{L1} and R^{L2}, or R^{M1} and R^{M2} form together with the carbon atom they are bonded to a group C═O, or C═S. Preferably, each R^{L1}, R^{L2}, R^{M1} and R^{M2} is independently H, C₁-C₃-alkyl, which groups are unsubstituted, or substituted or halogenated; or R^{L1} and R^{L2}, or R^{M1} and R^{M2} form together with the carbon atom they are bonded to a group C═O, or C═S.

Each R⁷, R⁸ is independently H; C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted, or substituted with one or more, same or different substitutent selected from halogen, CN and OH; benzyl or phenyl, which is unsubstituted or substituted with one or more, same or different substituents R¹¹, or NR⁷R⁸ may also form an N-bound, saturated 3- to 6-membered heterocycle, which in addition to the nitrogen atom may have 1 or 2 further heteroatoms or heteroatom moieties selected from O, S(═O)ₘ, NH, and N—C₁-C₆-alkyl, and wherein the N-bound heterocycle is unsubstituted or substituted with one or more, same or different substituents selected from halogen, C₁-C₄-alkyl, C₁-C₄-haloalkyl, C₁-C₄-alkoxy and C₁-C₄-haloalkoxy.

In one embodiment, each R⁷, R⁸ is independently H; C₁-C₃-alkyl, C₃-C₆-cycloalkyl, C₃-C₆-cycloalkyl-C₁-C₄-alkyl, which groups are unsubstituted, or substituted or halogenated; benzyl or phenyl, which is unsubstituted or substituted with one or more, same or different substituents selected from halogen, C₁-C₃-alkyl, C₁-C₃-alkoxy, C₁-C₃-haloalkoxy, or C₁-C₃-haloalkyl.

Each R^{X} is independently halogen, N₃, OH, CN, NO₂, SCN, SF₅;
C₁-C₆-alkyl, C₁-C₆-alkoxy, C₂-C₆-alkenyl, tri-C₁-C₆- alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, which groups are unsubstituted or substituted with CN or halogen;
C(═O)OR¹, NR²R³, C(═O)NR²R³, C(═O)R⁴, SO₂NR²R³, S(═O)ₘR¹, OR⁶, SR⁶, CH₂R⁶, OC(═O)R⁴, NR³C(═O)R⁴, OC(═O)OR¹, OC(═O)NR²R³, OC(═O)SR¹, OC(═S)NR²R³, OC(═S)SR¹, ONR²R³, ON═CR¹R⁴, N═CR¹R⁴, NNR², NC(═O)R⁹, SC(═O)SR¹, SC(═O)NR²R³, C(═S)R⁶, C(═S)OR⁴, C(═NR²)R⁴, C(R^{10a})═N—C(R^{10b});
phenyl, which is unsubstituted or substituted with one or more, same or different substituents R¹¹;
a 5- or 6-membered saturated, partially unsaturated, or fully unsaturated heterocyclic ring, wherein said heterocyclic ring comprises one or more, same or different heteroatoms O, N, or S, and is unsubstituted, or substituted with one or more, same or different substituents R³¹, and wherein said N- and S-atoms are independently oxidized, or non-oxidized; or
a group of formula (S)

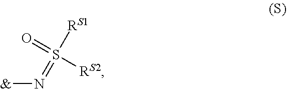

wherein each R^{S1}, R^{S2} is independently selected from C₁-C₆-alkyl, C₃-C₆-cycloalkyl, C₂-C₆-alkenyl, C₃-C₆-cycloalkenyl, C₂-C₆-alkynyl, C₃-C₆-cycloalkyl-C₁-C₃-alkyl, which groups are unsubstituted, or halogenated;
a 3- to 6-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said N- and S-atoms are independently oxidized, or non-oxidized; phenyl, which is unsubstituted, or substituted with one or more, same or different substituents selected from halogen, C₁-C₃-alkyl, C₁-C₃-alkoxy, C₁-C₃-haloalkyl, and C₁-C₃-haloalkoxy; or two substituents R^{S1}, R^{S2} form, together with the sulfur atom to which they are bound, a 5- or 6- membered saturated, partially unsaturated, or fully unsaturated heterocycle, which heterocycle is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said heterocycle comprises no, one or more, same or different heteroatoms O, N, or S in addition to the sulfur atom to which R^{S1} and R^{S2} are bound to.

Typically, each R^{X} is independently halogen, CN;
C₁-C₃-alkyl, C₁-C₃-alkoxy, C₃-C₆-cycloalkyl, C₃-C₆-cycloalkoxy, C₃-C₆-cycloalkyl-C₁-C₄-alkyl, which groups are unsubstituted or substituted with CN or halogen;
NR³C(═O)R⁹, C(R^{10a})═N—O(R^{10b});
phenyl, which is unsubstituted or substituted with one or more, same or different substituents R¹¹;
a 5- or 6-membered saturated, partially unsaturated, or fully unsaturated heterocyclic ring, wherein said heterocyclic ring comprises one or more, same or different heteroatoms O, N, or S, and is unsubstituted, or substituted with one or more, same or different substituents R³¹, and wherein said N- and S-atoms are independently oxidized, or non-oxidized; or
a group of formula (S),
wherein each R^{S1}, R^{S2} is independently selected from C₁-C₆-alkyl, C₃-C₆-cycloalkyl, which groups are unsubstituted, or halogenated;
or two substituents R^{S1}, R^{S2} form, together with the sulfur atom to which they are bound, a 5- or 6- membered saturated, partially unsaturated, or fully unsaturated heterocycle, which heterocycle is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said heterocycle comprises no, one or more, same or different heteroatoms O, N, or S in addition to the sulfur atom to which R^{S1} and R^{S2} are bound to.

In another embodiment, each R^{X} is independently halogen;
C₁-C₃-alkoxy, which groups are unsubstituted or substituted with CN or halogen;
NR³C(═O)R⁹, C(R^{10a})═N—O(R^{10b});
phenyl, which is unsubstituted or substituted with one or more, same or different substituents selected from halogen, C₁-C₃-alkyl, C₁-C₃-alkoxy, C₁-C₃-haloalkyl, and C₁-C₃-haloalkoxy;
a 5- or 6-membered saturated, partially unsaturated, or fully unsaturated heterocyclic ring, wherein said heterocyclic ring comprises one or more, same or different heteroatoms O, N, or S, and is unsubstituted, or substituted with one or more, same or different substituents selected from halogen, CN, C₁-C₃-alkyl, C₁-C₃-alkoxy, C₁-C₃-haloalkyl, and C₁-C₃-haloalkoxy, and wherein two substituents may form together with the carbon-atom to which they are bound a group (C═O), and wherein said N- and S-atoms are independently oxidized, or non-oxidized; or
a group of formula (S),
wherein each R^{S1}, R^{S2} is independently selected from C₁-C₃-alkyl, which groups are unsubstituted, or halogenated;
or two substituents R^{S1}, R^{S2} form, together with the sulfur atom to which they are bound, a 5- or 6- membered saturated, partially unsaturated, or fully unsaturated heterocycle, which heterocycle is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said heterocycle comprises no, one or more, same or different heteroatoms O, N, or S in addition to the sulfur atom to which R^{S1} and R^{S2} are bound to.

In another embodiment, each R^{X} is independently halogen;
C₁-C₃-alkyl, C₁-C₃-alkoxy, which groups are unsubstituted or substituted with CN or halogen (such as 1-cyanoisopropyl);
NR³C(═O)R⁹, C(R^{10a})═N—O(R^{10b});
phenyl, which is unsubstituted or halogenated;
a 5- or 6-membered saturated, partially unsaturated, or fully unsaturated heterocyclic ring, wherein said heterocyclic ring comprises one or more, same or different heteroatoms O, N, or S, and is unsubstituted, or substituted with one or more, same or different substituents selected from halogen, CN, C₁-C₃-alkyl, C₁-C₃-alkoxy, C₁-C₃-haloalkyl, and C₁-C₃-haloalkoxy, and wherein two substituents may form together with the carbon-atom to which they are bound a group (C═O), and wherein said N- and S-atoms are independently oxidized, or non-oxidized; or
a group of formula (S),
wherein each R^{S1}, R^{S2} is independently selected from C₁-C₃-alkyl, which groups are unsubstituted, or halogenated;
or two substituents R^{S1}, R^{S2} form, together with the sulfur atom to which they are bound, a 6-membered saturated, partially unsaturated, or fully unsaturated heterocycle, which heterocycle is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said heterocycle comprises no, one or more, same or different heteroatoms O, N, or S in addition to the sulfur atom to which R^{S1} and R^{S2} are bound to.

Preferably, R^{X} is phenyl, which is unsubstituted or substituted with halogen, preferably substituted with halogen, such as F.

In another embodiment, each R^{X} is independently halogen; C₃-C₆-cycloalkyl or phenyl, which groups are unsubstituted or substituted with halogen, preferably substituted with halogen, such as F;
C₁-C₃-alkoxy, which is substituted with CN;
   a 5- or 6-membered saturated, partially unsaturated, or fully unsaturated heterocyclic ring, wherein said heterocyclic ring comprises one or more, same or different heteroatoms O, N, or S, and is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein two substituents may form together with the carbon-atom to which they are bound a group (C═O); or
   a group of formula (S),
   wherein each R^{S1}, R^{S2} is independently selected from C₁-C₃-alkyl.

In another embodiment, each R^{X} is independently halogen; phenyl, which is unsubstituted or substituted with halogen, preferably substituted with halogen, such as F; or
a group of formula (S),
wherein each R^{S1}, R^{S2} is independently selected from C₁-C₃-alkyl.

R³¹ is halogen, N₃, OH, CN, NO₂, SCN, SF₅; C₁-C₆-alkyl, C₁-C₆-haloalkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₁-C₆-alkoxycarbonyl, C₃-C₆-cycloalkyl; C₃-C₆-cycloalkoxy, C₃-C₆-cycloalkyl-C₁-C₄-alkyl, C₃-C₆-cycloalkoxy-C₁-C₄ alkyl, phenyl, or a saturated, partially-, or fully unsaturated 5- or 6-membered heterocycle, wherein the cyclic moieties are unsubstituted or substituted with one or more, same or different substituents R¹¹; or two geminal substituents R³¹ form together with the atom to which they are bound a group ═O or ═S.

In one embodiment, R³¹ is halogen, CN, C₁-C₃-alkyl, C₁-C₃-haloalkyl, C₁-C₃-alkoxycarbonyl, or two geminal substituents form together with the atom to which they are bound a group ═O. In one embodiment, R³¹ is halogen, C₁-C₃-alkyl, C₁-C₃-haloalkyl, or two geminal substituents form together with the atom to which they are bound a group ═O.

In one embodiment, R³¹ is halogen, CN, C₁-C₃-alkyl, C₁-C₃-haloalkyl, C₁-C₃-alkoxycarbonyl, or two geminal substituents form together with the atom to which they are bound a group ═O.

In one embodiment, R³¹ is halogen, CN, C₁-C₃-alkyl, C₁-C₃-alkyl, C₁-C₃-haloalkyl, or two geminal substituents form together with the atom to which they are bound a group ═O.

Each R⁹ is independently C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with one or more, same or different substituents selected from CN and halogen. In one embodiment, each R⁹ is independently C₁-C₃-alkyl, or C₃-C₆-cycloalkyl, which groups are unsubstituted or substituted with one or more, same or different substituents selected from CN and halogen. In another embodiment, each R⁹ is independently C₃-C₆-cycloalkyl, which is unsubstituted or substituted with one or more, same or different substituents selected from CN. In another embodiment, R⁹ is 1-cyanocyclopropyl.

Each R^{10 a}is independently H, CN, OH, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen;
phenyl or benzyl, wherein the phenyl ring is unsubstituted or substituted with one or more, same or different substituents R¹¹. In one embodiment, R^{10a} is H, CN, C₁-C₃-alkyl, or C₁-C₃-haloalkyl. In another embodiment, R^{10a} is CN.

Each R^{10b} is independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with one or more, same or different substituents selected from halogen and CN; phenyl or benzyl, wherein the phenyl ring is unsubstituted or substituted with one or more, same or different substitutents R¹¹. In one embodiment, each R^{10b} is independently H, C₁-C₃-alkyl, or C₁-C₃-haloalkyl. In another embodiment, each R^{10b} is independently C₁-C₃-alkyl.

The index m is 0, 1, or 2. Typically, m is 0 or 2. In one embodiment, m is 2. In another embodiment, m is 0.

Preferably, compounds of formula (1) are compounds of formulae (II.1), (III.1), or (IV.1)

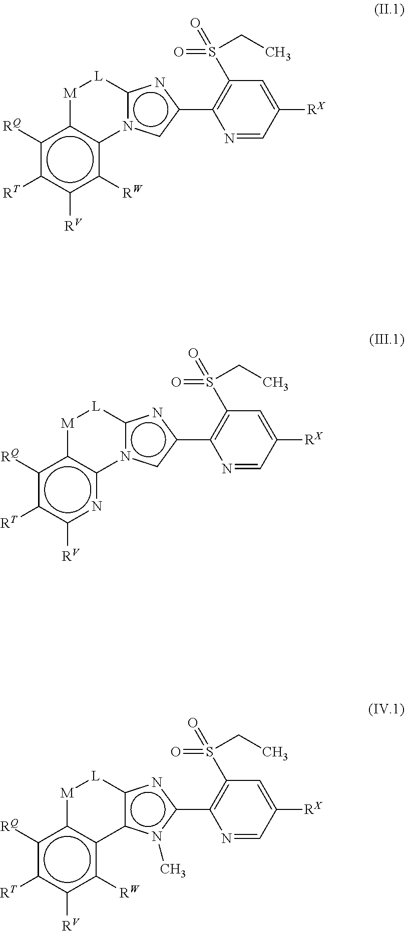

wherein all variables have a meaning as defined for formula (I). More preferably, compounds of formula (I) are compounds of formulae (II..1), or (IV.1).

Table A below contains combinations of meanings for variables M, L, and R^{X} in lines S-1 to S-205. The resective numbering S-1 to S-228 of the lines of Table A is used herein below as an abbreviation for the specific combination of meanings of the variables M, L, and R^{X} in this line.

Table B below contains combinations of meanings for variables R^{Q}, R^{T}, R^{V} and R^{W} in lines T-1 to T-29. The resective numbering T-1 to T-29 of the lines of Table B is used herein below as an abbreviation for the specific combination of meanings of the variables R^{Q}, R^{T}, R^{V} and RW in the line of Table C. Moreover, the meanings mentioned for the individual variables in Table A and Table B are per se, independently of the combination in which they are mentioned, a particularly preferred embodiment of the substituents in question.

TABLE A
assignment of lines S-1 to S-228 to combinations of M, L, R^{X}

| Line | M | L | R^{X} |
|---|---|---|---|
| S-1 | O | CH₂ | H |
| S-2 | O | CH₂ | CF₃ |
| S-3 | O | CH₂ | 4-fluorophenyl |
| S-4 | O | CH₂ | Br |
| S-5 | O | CH₂ | cyanomethyl |
| S-6 | O | CH₂ | 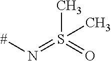 |
| S-7 | O | CH₂ | 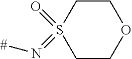 |
| S-8 | O | CH₂ | 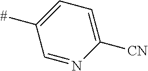 |
| S-9 | O | CH₂ | 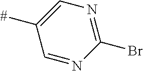 |
| S-10 | O | CH₂ | 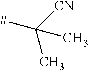 |
| S-11 | O | CH₂ | cyanomethyl |
| S-12 | O | CH₂ | 1-cyano-1- |
| | | | methyl-ethoxy |
| S-13 | O | CH₂ | 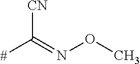 |
| S-14 | O | CH₂ | 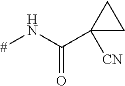 |
| S-15 | O | CH₂ | 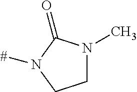 |
| S-16 | O | CH₂ | 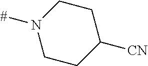 |
| S-17 | O | CH₂ | 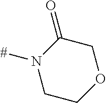 |
| S-18 | O | CH₂ | cyclopropyl |
| S-19 | O | CH₂ | 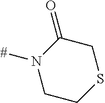 |
| S-20 | O | C(CH₃)₂ | H |
| S-21 | O | C(CH₃)₂ | CF₃ |
| S-22 | O | C(CH₃)₂ | 4-fluorophenyl |
| S-23 | O | C(CH₃)₂ | Br |
| S-24 | O | C(CH₃)₂ | cyanomethyl |
| S-25 | O | C(CH₃)₂ | 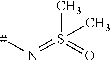 |
| S-26 | O | C(CH₃)₂ | 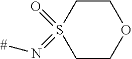 |
| S-27 | O | C(CH₃)₂ | 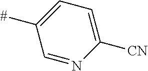 |
| S-28 | O | C(CH₃)₂ | 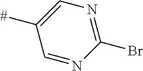 |
| S-29 | O | C(CH₃)₂ | 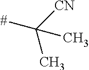 |
| S-30 | O | C(CH₃)₂ | cyanomethyl |
| S-31 | O | C(CH₃)₂ | 1-cyano-1- |
| | | | methyl-ethoxy |
| S-32 | O | C(CH₃)₂ | 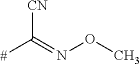 |
| S-33 | O | C(CH₃)₂ | 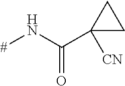 |
| S-34 | O | C(CH₃)₂ | 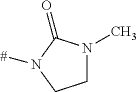 |
| S-35 | O | C(CH₃)₂ | 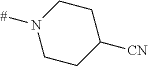 |
| S-36 | O | C(CH₃)₂ | 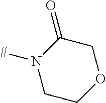 |
| S-37 | O | C(CH₃)₂ | cyclopropyl |
| S-38 | O | C(CH₃)₂ | 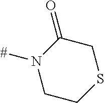 |
| S-39 | O | C(═O) | H |
| S-40 | O | C(═O) | CF₃ |
| S-41 | O | C(═O) | 4-fluorophenyl |
| S-42 | O | C(═O) | Br |
| S-43 | O | C(═O) | cyanomethyl |
| S-44 | O | C(═O) | 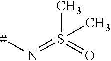 |
| S-45 | O | C(═O) | 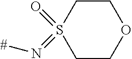 |
| S-46 | O | C(═O) | 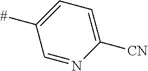 |
| S-47 | O | C(═O) | 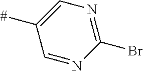 |
| S-48 | O | C(═O) | 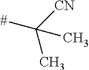 |
| S-49 | O | C(═O) | cyanomethyl |
| S-50 | O | C(═O) | 1-cyano-1- |
| | | | methyl-ethoxy |
| S-51 | O | C(═O) | 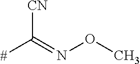 |
| S-52 | O | C(═O) | 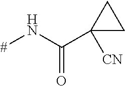 |
| S-53 | O | C(═O) | 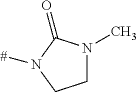 |
| S-54 | O | C(═O) | 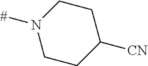 |
| S-55 | O | C(═O) | 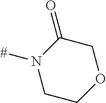 |
| S-56 | O | C(═O) | cyclopropyl |
| S-57 | O | C(═O) | 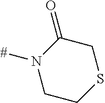 |
| S-58 | S | CH₂ | H |
| S-59 | S | CH₂ | CF₃ |
| S-60 | S | CH₂ | 4-fluorophenyl |
| S-61 | S | CH₂ | Br |
| S-62 | S | CH₂ | cyanomethyl |
| S-63 | S | CH₂ | 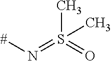 |
| S-64 | S | CH₂ | 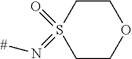 |
| S-65 | S | CH₂ | 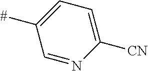 |
| S-66 | S | CH₂ | 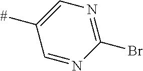 |
| S-67 | S | CH₂ | 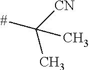 |
| S-68 | S | CH₂ | cyanomethyl |
| S-69 | S | CH₂ | 1-cyano-1- |
| | | | methyl-ethoxy |
| S-70 | S | CH₂ | 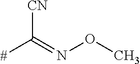 |
| S-71 | S | CH₂ | 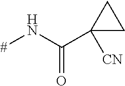 |
| S-72 | S | CH₂ | 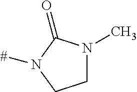 |
| S-73 | S | CH₂ | 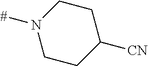 |
| S-74 | S | CH₂ | 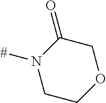 |
| S-75 | S | CH₂ | cyclopropyl |
| S-76 | S | CH₂ | 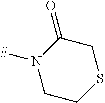 |
| S-77 | S | C(CH₃)₂ | H |
| S-78 | S | C(CH₃)₂ | CF |
| S-79 | S | C(CH₃)₂ | 4-fluorophenyl |
| S-80 | S | C(CH₃)₂ | Br |
| S-81 | S | C(CH₃)₂ | cyanomethyl |
| S-82 | S | C(CH₃)₂ | 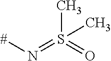 |
| S-83 | S | C(CH₃)₂ | 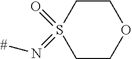 |
| S-84 | S | C(CH₃)₂ | 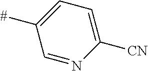 |
| S-85 | S | C(CH₃)₂ | 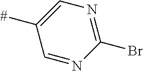 |
| S-86 | S | C(CH₃)₂ | 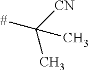 |
| S-87 | S | C(CH₃)₂ | cyanomethyl |
| S-88 | S | C(CH₃)₂ | 1-cyano-1- |
| | | | methyl-ethoxy |
| S-89 | S | C(CH₃)₂ | 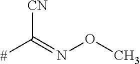 |
| S-90 | S | C(CH₃)₂ | 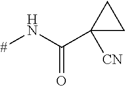 |
| S-91 | S | C(CH₃)₂ | 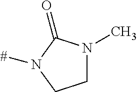 |
| S-92 | S | C(CH₃)₂ | 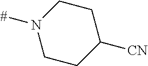 |
| S-93 | S | C(CH₃)₂ | 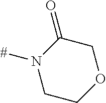 |
| S-94 | S | C(CH₃)₂ | cyclopropyl |
| S-95 | S | C(CH₃)₂ | 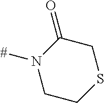 |
| S-96 | S | C(═O) | H |
| S-97 | S | C(═O) | CF₃ |
| S-98 | S | C(═O) | 4-fluorophenyl |
| S-99 | S | C(═O) | Br |
| S-100 | S | C(═O) | cyanomethyl |
| S-101 | S | C(═O) | 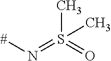 |
| S-102 | S | C(═O) | 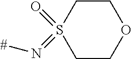 |
| S-103 | S | C(═O) | 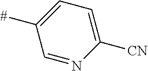 |
| S-104 | S | C(═O) | 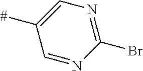 |
| S-105 | S | C(═O) | 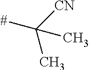 |
| S-106 | S | C(═O) | cyanomethyl |
| S-107 | S | C(═O) | 1-cyano-1- |
| | | | methyl-ethoxy |
| S-108 | S | C(═O) | 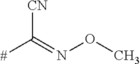 |
| S-109 | S | C(═O) | 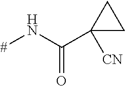 |
| S-110 | S | C(═O) | 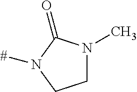 |
| S-111 | S | C(═O) | 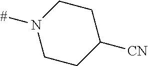 |
| S-112 | S | C(═O) | 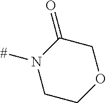 |
| S-113 | S | C(═O) | cyclopropyl |
| S-114 | S | C(═O) | 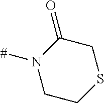 |
| S-115 | CH₂ | CH₂ | H |
| S-116 | CH₂ | CH₂ | CF₃ |
| S-117 | CH₂ | O | 4-fluorophenyl |
| S-118 | CH₂ | O | Br |
| S-119 | CH₂ | O | cyanomethyl |
| S-120 | CH₂ | O | 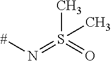 |
| S-121 | CH₂ | O | 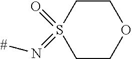 |
| S-122 | CH₂ | O | 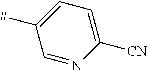 |
| S-123 | CH₂ | O | 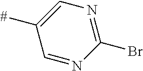 |
| S-124 | CH₂ | O | 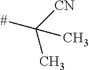 |
| S-125 | CH₂ | O | cyanomethyl |
| S-126 | CH₂ | O | 1-cyano-1- |
| | | | methyl-ethoxy |
| S-127 | CH₂ | O | 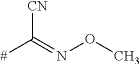 |
| S-128 | CH₂ | O | 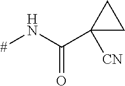 |
| S-129 | CH₂ | O | 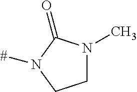 |
| S-130 | CH₂ | O | 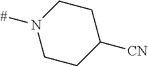 |
| S-131 | CH₂ | O | 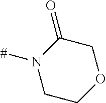 |
| S-132 | CH₂ | O | cyclopropyl |
| S-133 | CH₂ | O | 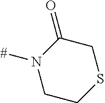 |
| S-134 | C(CH₃)₂ | O | H |
| S-135 | C(CH₃)₂ | O | CF₃ |
| S-136 | C(CH₃)₂ | O | 4-fluorophenyl |
| S-137 | C(CH₃)₂ | O | Br |
| S-138 | C(CH₃)₂ | O | cyanomethyl |
| S-139 | C(CH₃)₂ | O | 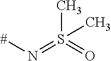 |
| S-140 | C(CH₃)₂ | O | 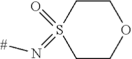 |
| S-141 | C(CH₃)₂ | O | 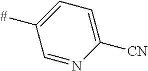 |
| S-142 | C(CH₃)₂ | O | 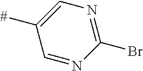 |
| S-143 | C(CH₃)₂ | O | 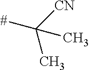 |
| S-144 | C(CH₃)₂ | O | cyanomethyl |
| S-145 | C(CH₃)₂ | O | 1-cyano-1- |
| | | | methyl-ethoxy |
| S-146 | C(CH₃)₂ | O | 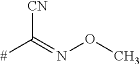 |
| S-147 | C(CH₃)₂ | O | 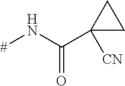 |
| S-148 | C(CH₃)₂ | O | 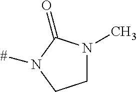 |
| S-149 | C(CH₃)₂ | O | 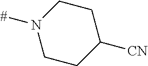 |
| S-150 | C(CH₃)₂ | O | 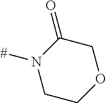 |
| S-151 | C(CH₃)₂ | O | cyclopropyl |
| S-152 | C(CH₃)₂ | O | 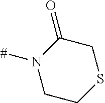 |
| S-153 | C(═O) | O | H |
| S-154 | C(═O) | O | CF₃ |
| S-155 | C(═O) | O | 4-fluorophenyl |
| S-156 | C(═O) | O | Br |
| S-157 | C(═O) | O | cyanomethyl |
| S-158 | C(═O) | O | 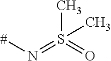 |
| S-159 | C(═O) | O | 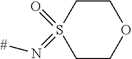 |
| S-160 | C(═O) | O | 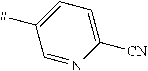 |
| S-161 | C(═O) | O | 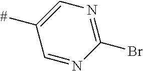 |
| S-162 | C(═O) | O | 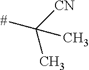 |
| S-163 | C(═O) | O | cyanomethyl |
| S-164 | C(═O) | O | 1-cyano-1- |
| | | | methyl-ethoxy |
| S-165 | C(═O) | O | 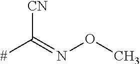 |
| S-166 | C(═O) | O | 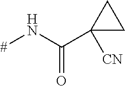 |
| S-167 | C(═O) | O | 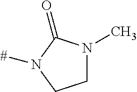 |
| S-168 | C(═O) | O | 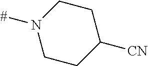 |
| S-169 | C(═O) | O | 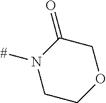 |
| S-170 | C(═O) | O | cyclopropyl |
| S-171 | C(═O) | O | 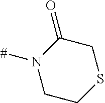 |
| S-172 | CH₂ | S | H |
| S-173 | CH₂ | S | CF₃ |
| S-174 | CH₂ | S | 4-fluorophenyl |
| S-175 | CH₂ | S | Br |
| S-176 | CH₂ | S | cyanomethyl |
| S-177 | CH₂ | S | 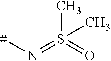 |
| S-178 | CH₂ | S | 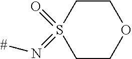 |
| S-179 | CH₂ | S | 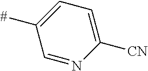 |
| S-180 | CH₂ | S | 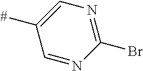 |
| S-181 | CH₂ | S | 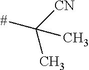 |
| S-182 | CH₂ | S | cyanomethyl |
| S-183 | CH₂ | S | 1-cyano-1- |
| | | | methyl-ethoxy |
| S-184 | CH₂ | S | 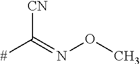 |
| S-185 | CH₂ | S | 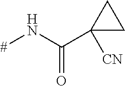 |
| S-186 | CH₂ | S | 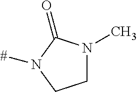 |
| S-187 | CH₂ | S | 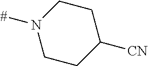 |
| S-188 | CH₂ | S | 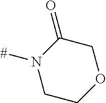 |
| S-189 | CH₂ | S | cyclopropyl |
| S-190 | CH₂ | S | 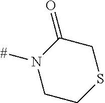 |
| S-191 | C(CH₃)₂ | S | H |
| S-192 | C(CH₃)₂ | S | CF₃ |
| S-193 | C(CH₃)₂ | S | 4-fluorophenyl |
| S-194 | C(CH₃)₂ | S | Br |
| S-195 | C(CH₃)₂ | S | cyanomethyl |
| S-196 | C(CH₃)₂ | S | 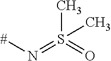 |
| S-197 | C(CH₃)₂ | S | 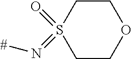 |
| S-198 | C(CH₃)₂ | S | 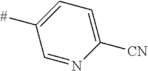 |
| S-199 | C(CH₃)₂ | S | 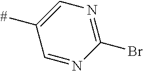 |
| S-200 | C(CH₃)₂ | S | 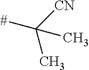 |
| S-201 | C(CH₃)₂ | S | cyanomethyl |
| S-202 | C(CH₃)₂ | S | 1-cyano-1- |
| | | | methyl-ethoxy |
| S-203 | C(CH₃)₂ | S | 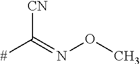 |
| S-204 | C(CH₃)₂ | S | 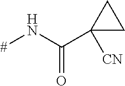 |
| S-205 | C(CH₃)₂ | S | 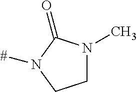 |
| S-206 | C(CH₃)₂ | S | 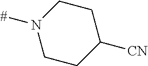 |
| S-207 | C(CH₃)₂ | S | 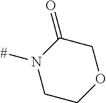 |
| S-208 | C(CH₃)₂ | S | cyclopropyl |
| S-209 | C(CH₃)₂ | S | 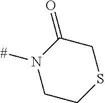 |
| S-210 | C(═O) | S | H |
| S-211 | C(═O) | S | CF₃ |
| S-212 | C(═O) | S | 4-fluorophenyl |
| S-213 | C(═O) | S | Br |
| S-214 | C(═O) | S | cyanomethyl |
| S-215 | C(═O) | S | 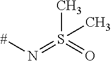 |
| S-216 | C(═O) | S | 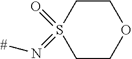 |
| S-217 | C(═O) | S | 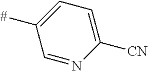 |
| S-218 | C(═O) | S | 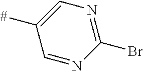 |
| S-219 | C(═O) | S | 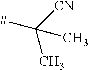 |
| S-220 | C(═O) | S | cyanomethyl |
| S-221 | C(═O) | S | 1-cyano-1- |
| | | | methyl-ethoxy |
| S-222 | C(═O) | S | 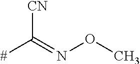 |
| S-223 | C(═O) | S | 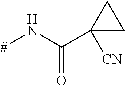 |
| S-224 | C(═O) | S | 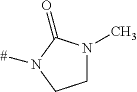 |
| S-225 | C(═O) | S | 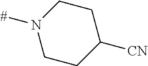 |
| S-226 | C(═O) | C | 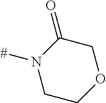 |
| S-227 | C(═O) | S | cyclopropyl |
| S-228 | C(═O) | S | 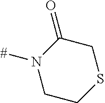 |

TABLE B
assignment of lines T-1 to T-37 to
combinations of R^{Q}, R^{T}, R^{V} and R^{W}.

| Line | R^{Q} | R^{T} | R^{V} | R^{W} |
|---|---|---|---|---|
| T-1 | H | H | H | H |
| T-2 | CF₃ | H | H | H |
| T-3 | OCF₃ | H | H | H |
| T-4 | CH₃ | H | H | H |
| T-5 | OCH₃ | H | H | H |
| T-6 | H | CF₃ | H | H |
| T-7 | H | OCF₃ | H | H |
| T-8 | H | CH₃ | H | H |
| T-9 | H | OCH₃ | H | H |
| T-10 | H | H | CF₃ | H |
| T-11 | H | H | OCF₃ | H |
| T-12 | H | H | CH₃ | H |
| T-13 | H | H | OCH₃ | H |
| T-14 | H | H | H | CF₃ |
| T-15 | H | H | H | OCF₃ |
| T-16 | H | H | H | CH₃ |
| T-17 | H | H | H | OCH₃ |
| T-18 | CF₃ | CF₃ | H | H |
| T-19 | CF₃ | H | CF₃ | H |
| T-20 | CF₃ | H | H | CF₃ |
| T-21 | H | CF₃ | CF₃ | H |
| T-22 | H | CF₃ | H | CF₃ |
| T-23 | H | H | CF₃ | CF₃ |
| T-24 | OCF₃ | OCF₃ | H | H |
| T-25 | OCF₃ | H | OCF₃ | H |
| T-26 | OCF₃ | H | H | OCF₃ |
| T-27 | H | OCF₃ | OCF₃ | H |
| T-28 | H | OCF₃ | H | OCF₃ |
| T-29 | H | H | OCF₃ | OCF₃ |
| T-30 | CF₃ | CF₃ | CF₃ | H |
| T-31 | CF₃ | H | CF₃ | CF₃ |
| T-32 | CF₃ | CF₃ | H | CF₃ |
| T-33 | H | CF₃ | CF₃ | CF₃ |
| T-34 | OCF₃ | OCF₃ | OCF₃ | H |
| T-35 | OCF₃ | H | OCF₃ | OCF₃ |
| T-36 | OCF₃ | OCF₃ | H | OCF₃ |
| T-37 | H | OCF₃ | OCF₃ | OCF₃ |

The following Tables 1 to 111 represent preferred embodiments of combinations of variables R^{Q}, R^{T}, R^{V}, R^{W}, L, M, and R^{X} with formulae (II.1), (III.1), or (IV.1). In case the variable does not occur in the respective formula, it should be clear that no definition is required; in other words the definition of the non-existing variable is not considered.

Table 1: Compound of formula (II.1), wherein R^{Q}, R^{T}, R^{V} and R^{W} are as defined as in line T-1 of Table B, and wherein the definition of the variables M, L, and R^{X} is as defined in a line of Table A.

Table 2: Compound of formula (II.1), wherein R^{Q}, R^{T}, R^{V} and R^{W} are as defined as in line T2 of Table B, and wherein the definition of the variables M, L, and R^{X} is as defined in a line of Table A.

Table 3: Compound of formula (II.1), wherein R^{Q}, R^{T}, R^{V} and R^{W} are as defined as in line T3 of Table B, and wherein the definition of the variables M, L, and R^{X} is as defined in a line of Table A.

Table 4: Compound of formula (II.1), wherein R^{Q}, R^{T}, R^{V} and R^{W} are as defined as in line T4 of Table B, and wherein the definition of the variables M, L, and R^{X} is as defined in a line of Table A.

Table 5: Compound of formula (II.1), wherein R^{Q}, R^{T}, R^{V} and R^{W} are as defined as in line T5 of Table B, and wherein the definition of the variables M, L, and R^{X} is as defined in a line of Table A.

Table 6: Compound of formula (II.1), wherein R^{Q}, R^{T}, R^{V} and R^{W} are as defined as in line T6 of Table B, and wherein the definition of the variables M, L, and R^{X} is as defined in a line of Table A.

Table 7: Compound of formula (II.1), wherein R^{Q}, R^{T}, R^{V} and R^{W} are as defined as in line T7 of Table B, and wherein the definition of the variables M, L, and R^{X} is as defined in a line of Table A.

Table 8: Compound of formula (II.1), wherein R^{Q}, R^{T}, R^{V} and R^{W} are as defined as in line T8 of Table B, and wherein the definition of the variables M, L, and R^{X} is as defined in a line of Table A.

Table 9: Compound of formula (II.1), wherein R^{Q}, R^{T}, R^{V} and R^{W} are as defined as in line T9 of Table B, and wherein the definition of the variables M, L, and R^{X} is as defined in a line of Table A.

Table 10: Compound of formula (II.1), wherein R^{Q}, R^{T}, R^{V} and R^{W} are as defined as in line T10 of Table B, and wherein the definition of the variables M, L, and R^{X} is as defined in a line of Table A.

Table 11: Compound of formula (II.1), wherein R^{Q}, R^{T}, R^{V} and R^{W} are as defined as in line T11 of Table B, and wherein the definition of the variables M, L, and R^{X} is as defined in a line of Table A.

Table 12: Compound of formula (II.1), wherein R^{Q}, R^{T}, R^{V} and R^{W} are as defined as in line T12 of Table B, and wherein the definition of the variables M, L, and R^{X} is as defined in a line of Table A.

Table 13: Compound of formula (II.1), wherein R^{Q}, R^{T}, R^{V} and R^{W} are as defined as in line T13 of Table B, and wherein the definition of the variables M, L, and R^{X} is as defined in a line of Table A.

Table 14: Compound of formula (II.1), wherein R^{Q}, R^{T}, R^{V} and R^{W} are as defined as in line T14 of Table B, and wherein the definition of the variables M, L, and R^{X} is as defined in a line of Table A.

Table 15: Compound of formula (II.1), wherein R^{Q}, R^{T}, R^{V} and R^{W} are as defined as in line T15 of Table B, and wherein the definition of the variables M, L, and R^{X} is as defined in a line of Table A.

Table 16: Compound of formula (II.1), wherein R^{Q}, R^{T}, R^{V} and R^{W} are as defined as in line T16 of Table B, and wherein the definition of the variables M, L, and R^{X} is as defined in a line of Table A.

Table 17: Compound of formula (II.1), wherein R^{Q}, R^{T}, R^{V} and R^{W} are as defined as in line T17 of Table B, and wherein the definition of the variables M, L, and R^{X} is as defined in a line of Table A.

Table 18: Compound of formula (II.1), wherein R^{Q}, R^{T}, R^{V} and R^{W} are as defined as in line T18 of Table B, and wherein the definition of the variables M, L, and R^{X} is as defined in a line of Table A.

Table 19: Compound of formula (II.1), wherein R^{Q}, R^{T}, R^{V} and R^{W} are as defined as in line T19 of Table B, and wherein the definition of the variables M, L, and R^{X} is as defined in a line of Table A.

Table 20: Compound of formula (II.1), wherein R^{Q}, R^{T}, R^{V} and R^{W} are as defined as in line T20 of Table B, and wherein the definition of the variables M, L, and R^{X} is as defined in a line of Table A.

Table 21: Compound of formula (II.1), wherein R^{Q}, R^{T}, R^{V} and R^{W} are as defined as in line T21 of Table B, and wherein the definition of the variables M, L, and R^{X} is as defined in a line of Table A.

Table 22: Compound of formula (II.1), wherein R^{Q}, R^{T}, R^{V} and R^{W} are as defined as in line T22 of Table B, and wherein the definition of the variables M, L, and R^{X} is as defined in a line of Table A.

Table 23: Compound of formula (II.1), wherein R^{Q}, R^{T}, R^{V} and R^{W} are as defined as in line T23 of Table B, and wherein the definition of the variables M, L, and R^{X} is as defined in a line of Table A.

Table 24: Compound of formula (II.1), wherein R^{Q}, R^{T}, R^{V} and R^{W} are as defined as in line T24 of Table B, and wherein the definition of the variables M, L, and R^{X} is as defined in a line of Table A.

Table 25: Compound of formula (II.1), wherein R^{Q}, R^{T}, R^{V} and R^{W} are as defined as in line T25 of Table B, and wherein the definition of the variables M, L, and R^{X} is as defined in a line of Table A.

Table 26: Compound of formula (II.1), wherein R^{Q}, R^{T}, R^{V} and R^{W} are as defined as in line T26 of Table B, and wherein the definition of the variables M, L, and R^{X} is as defined in a line of Table A.

Table 27: Compound of formula (II.1), wherein R^{Q}, R^{T}, R^{V} and R^{W} are as defined as in line T27 of Table B, and wherein the definition of the variables M, L, and R^{X} is as defined in a line of Table A.

Table 28: Compound of formula (II.1), wherein R^{Q}, R^{T}, R^{V} and R^{W} are as defined as in line T28 of Table B, and wherein the definition of the variables M, L, and R^{X} is as defined in a line of Table A.

Table 29: Compound of formula (II.1), wherein R^{Q}, R^{T}, R^{V} and R^{W} are as defined as in line T29 of Table B, and wherein the definition of the variables M, L, and R^{X} is as defined in a line of Table A.

Table 30: Compound of formula (II.1), wherein R^{Q}, R^{T}, R^{V} and R^{W} are as defined as in line T30 of Table B, and wherein the definition of the variables M, L, and R^{X} is as defined in a line of Table A.

Table 31: Compound of formula (II.1), wherein R^{Q}, R^{T}, R^{V} and R^{W} are as defined as in line T31 of Table B, and wherein the definition of the variables M, L, and R^{X} is as defined in a line of Table A.

Table 32: Compound of formula (II.1), wherein R^{Q}, R^{T}, R^{V} and R^{W} are as defined as in line T32 of Table B, and wherein the definition of the variables M, L, and R^{X} is as defined in a line of Table A.

Table 33: Compound of formula (II.1), wherein R^{Q}, R^{T}, R^{V} and R^{W} are as defined as in line T33 of Table B, and wherein the definition of the variables M, L, and R^{X} is as defined in a line of Table A.

Table 34: Compound of formula (II.1), wherein R^{Q}, R^{T}, R^{V} and R^{W} are as defined as in line T34 of Table B, and wherein the definition of the variables M, L, and R^{X} is as defined in a line of Table A.

Table 35: Compound of formula (II.1), wherein R^{Q}, R^{T}, R^{V} and R^{W} are as defined as in line T35 of Table B, and wherein the definition of the variables M, L, and R^{X} is as defined in a line of Table A.

Table 36: Compound of formula (II.1), wherein R^{Q}, R^{T}, R^{V} and R^{W} are as defined as in line T36 of Table B, and wherein the definition of the variables M, L, and R^{X} is as defined in a line of Table A.

Table 37: Compound of formula (II.1), wherein R^{Q}, R^{T}, R^{V} and R^{W} are as defined as in line T37 of Table B, and wherein the definition of the variables M, L, and R^{X} is as defined in a line of Table A.

Table 38: Compound of formula (III.1), wherein R^{Q}, R^{T}, R^{V} are as defined as in line T-1 of Table B, and wherein the definition of the variables M, L, and R^{X} is as defined in a line of Table A.

Table 39: Compound of formula (III.1), wherein R^{Q}, R^{T}, R^{V} are as defined as in line T2 of Table B, and wherein the definition of the variables M, L, and R^{X} is as defined in a line of Table A.

Table 40: Compound of formula (III.1), wherein R^{Q}, R^{T}, R^{V} are as defined as in line T3 of Table B, and wherein the definition of the variables M, L, and R^{X} is as defined in a line of Table A.

Table 41: Compound of formula (III.1), wherein R^{Q}, R^{T}, R^{V} are as defined as in line T4 of Table B, and wherein the definition of the variables M, L, and R^{X} is as defined in a line of Table A.

Table 42: Compound of formula (III.1), wherein R^{Q}, R^{T}, R^{V} are as defined as in line T5 of Table B, and wherein the definition of the variables M, L, and R^{X} is as defined in a line of Table A.

Table 43: Compound of formula (III.1), wherein R^{Q}, R^{T}, R^{V} are as defined as in line T6 of Table B, and wherein the definition of the variables M, L, and R^{X} is as defined in a line of Table A.

Table 44: Compound of formula (III.1), wherein R^{Q}, R^{T}, R^{V} are as defined as in line T7 of Table B, and wherein the definition of the variables M, L, and R^{X} is as defined in a line of Table A.

Table 45: Compound of formula (III.1), wherein R^{Q}, R^{T}, R^{V} are as defined as in line T8 of Table B, and wherein the definition of the variables M, L, and R^{X} is as defined in a line of Table A.

Table 46: Compound of formula (III.1), wherein R^{Q}, R^{T}, R^{V} are as defined as in line T9 of Table B, and wherein the definition of the variables M, L, and R^{X} is as defined in a line of Table A.

Table 47: Compound of formula (III.1), wherein R^{Q}, R^{T}, R^{V} are as defined as in line T10 of Table B, and wherein the definition of the variables M, L, and R^{X} is as defined in a line of Table A.

Table 48: Compound of formula (III.1), wherein R^{Q}, R^{T}, R^{V} are as defined as in line T11 of Table B, and wherein the definition of the variables M, L, and R^{X} is as defined in a line of Table A.

Table 49: Compound of formula (III.1), wherein R^{Q}, R^{T}, R^{V} are as defined as in line T12 of Table B, and wherein the definition of the variables M, L, and R^{X} is as defined in a line of Table A.

Table 50: Compound of formula (III.1), wherein R^{Q}, R^{T}, R^{V} are as defined as in line T13 of Table B, and wherein the definition of the variables M, L, and R^{X} is as defined in a line of Table A.

Table 51: Compound of formula (III.1), wherein R^{Q}, R^{T}, R^{V} are as defined as in line T14 of Table B, and wherein the definition of the variables M, L, and R^{X} is as defined in a line of Table A.

Table 52: Compound of formula (III.1), wherein R^{Q}, R^{T}, R^{V} are as defined as in line T15 of Table B, and wherein the definition of the variables M, L, and R^{X} is as defined in a line of Table A.

Table 53: Compound of formula (III.1), wherein R^{Q}, R^{T}, R^{V} are as defined as in line T16 of Table B, and wherein the definition of the variables M, L, and R^{X} is as defined in a line of Table A.

Table 54: Compound of formula (III.1), wherein R^{Q}, R^{T}, R^{V} are as defined as in line T17 of Table B, and wherein the definition of the variables M, L, and R^{X} is as defined in a line of Table A.

Table 55: Compound of formula (III.1), wherein R^{Q}, R^{T}, R^{V} are as defined as in line T18 of Table B, and wherein the definition of the variables M, L, and R^{X} is as defined in a line of Table A.

Table 56: Compound of formula (III.1), wherein R^{Q}, R^{T}, R^{V} are as defined as in line T19 of Table B, and wherein the definition of the variables M, L, and R^{X} is as defined in a line of Table A.

Table 57: Compound of formula (III.1), wherein R^{Q}, R^{T}, R^{V} are as defined as in line T20 of Table B, and wherein the definition of the variables M, L, and R^{X} is as defined in a line of Table A.

Table 58: Compound of formula (III.1), wherein R^{Q}, R^{T}, R^{V} are as defined as in line T21 of Table B, and wherein the definition of the variables M, L, and R^{X} is as defined in a line of Table A.

Table 59: Compound of formula (III.1), wherein R^{Q}, R^{T}, R^{V} are as defined as in line T22 of Table B, and wherein the definition of the variables M, L, and R^{X} is as defined in a line of Table A.

Table 60: Compound of formula (III.1), wherein R^{Q}, R^{T}, R^{V} are as defined as in line T23 of Table B, and wherein the definition of the variables M, L, and R^{X} is as defined in a line of Table A.

Table 61: Compound of formula (III.1), wherein R^{Q}, R^{T}, R^{V} are as defined as in line T24 of Table B, and wherein the definition of the variables M, L, and R^{X} is as defined in a line of Table A.

Table 62: Compound of formula (III.1), wherein R^{Q}, R^{T}, R^{V} are as defined as in line T25 of Table B, and wherein the definition of the variables M, L, and R^{X} is as defined in a line of Table A.

Table 63: Compound of formula (III.1), wherein R^{Q}, R^{T}, R^{V} are as defined as in line T26 of Table B, and wherein the definition of the variables M, L, and R^{X} is as defined in a line of Table A.

Table 64: Compound of formula (III.1), wherein R^{Q}, R^{T}, R^{V} are as defined as in line T27 of Table B, and wherein the definition of the variables M, L, and R^{X} is as defined in a line of Table A.

Table 65: Compound of formula (III.1), wherein R^{Q}, R^{T}, R^{V} are as defined as in line T28 of Table B, and wherein the definition of the variables M, L, and R^{X} is as defined in a line of Table A.

Table 66: Compound of formula (III.1), wherein R^{Q}, R^{T}, R^{V} are as defined as in line T29 of Table B, and wherein the definition of the variables M, L, and R^{X} is as defined in a line of Table A.

Table 67: Compound of formula (III.1), wherein R^{Q}, R^{T}, R^{V} are as defined as in line T30 of Table B, and wherein the definition of the variables M, L, and R^{X} is as defined in a line of Table A.

Table 68: Compound of formula (III.1), wherein R^{Q}, R^{T}, R^{V} are as defined as in line T31 of Table B, and wherein the definition of the variables M, L, and R^{X} is as defined in a line of Table A.

Table 69: Compound of formula (III.1), wherein R^{Q}, R^{T}, R^{V} are as defined as in line T32 of Table B, and wherein the definition of the variables M, L, and R^{X} is as defined in a line of Table A.

Table 70: Compound of formula (III.1), wherein R^{Q}, R^{T}, R^{V} are as defined as in line T33 of Table B, and wherein the definition of the variables M, L, and R^{X} is as defined in a line of Table A.

Table 71: Compound of formula (III.1), wherein R^{Q}, R^{T}, R^{V} are as defined as in line T34 of Table B, and wherein the definition of the variables M, L, and R^{X} is as defined in a line of Table A.

Table 72: Compound of formula (III.1), wherein R^{Q}, R^{T}, R^{V} are as defined as in line T35 of Table B, and wherein the definition of the variables M, L, and R^{X} is as defined in a line of Table A.

Table 73: Compound of formula (III.1), wherein R^{Q}, R^{T}, R^{V} are as defined as in line T36 of Table B, and wherein the definition of the variables M, L, and R^{X} is as defined in a line of Table A.

Table 74: Compound of formula (III.1), wherein R^{Q}, R^{T}, R^{V} are as defined as in line T37 of Table B, and wherein the definition of the variables M, L, and R^{X} is as defined in a line of Table A.

Table 75: Compound of formula (IV.1), wherein R^{Q}, R^{T}, R^{V} and R^{W} are as defined as in line T1 of Table B, and wherein the definition of the variables M, L, and R^{X} is as defined in a line of Table A.

Table 76: Compound of formula (IV.1), wherein R^{Q}, R^{T}, R^{V} and R^{W} are as defined as in line T2 of Table B, and wherein the definition of the variables M, L, and R^{X} is as defined in a line of Table A.

Table 77: Compound of formula (IV.1), wherein R^{Q}, R^{T}, R^{V} and R^{W} are as defined as in line T3 of Table B, and wherein the definition of the variables M, L, and R^{X} is as defined in a line of Table A.

Table 78: Compound of formula (IV.1), wherein R^{Q}, R^{T}, R^{V} and R^{W} are as defined as in line T4 of Table B, and wherein the definition of the variables M, L, and R^{X} is as defined in a line of Table A.

Table 79: Compound of formula (IV.1), wherein R^{Q}, R^{T}, R^{V} and R^{W} are as defined as in line T5 of Table B, and wherein the definition of the variables M, L, and R^{X} is as defined in a line of Table A.

Table 80: Compound of formula (IV.1), wherein R^{Q}, R^{T}, R^{V} and R^{W} are as defined as in line T6 of Table B, and wherein the definition of the variables M, L, and R^{X} is as defined in a line of Table A.

Table 81: Compound of formula (IV.1), wherein R^{Q}, R^{T}, R^{V} and R^{W} are as defined as in line T7 of Table B, and wherein the definition of the variables M, L, and R^{X} is as defined in a line of Table A.

Table 82: Compound of formula (IV.1), wherein R^{Q}, R^{T}, R^{V} and R^{W} are as defined as in line T8 of Table B, and wherein the definition of the variables M, L, and R^{X} is as defined in a line of Table A.

Table 83: Compound of formula (IV.1), wherein R^{Q}, R^{T}, R^{V} and R^{W} are as defined as in line T9 of Table B, and wherein the definition of the variables M, L, and R^{X} is as defined in a line of Table A.

Table 84: Compound of formula (IV.1), wherein R^{Q}, R^{T}, R^{V} and R^{W} are as defined as in line T10 of Table B, and wherein the definition of the variables M, L, and R^{X} is as defined in a line of Table A.

Table 85: Compound of formula (IV.1), wherein R^{Q}, R^{T}, R^{V} and R^{W} are as defined as in line T11 of Table B, and wherein the definition of the variables M, L, and R^{X} is as defined in a line of Table A.

Table 86: Compound of formula (IV.1), wherein R^{Q}, R^{T}, R^{V} and R^{W} are as defined as in line T12 of Table B, and wherein the definition of the variables M, L, and R^{X} is as defined in a line of Table A.

Table 87: Compound of formula (IV.1), wherein R^{Q}, R^{T}, R^{V} and R^{W} are as defined as in line T13 of Table B, and wherein the definition of the variables M, L, and R^{X} is as defined in a line of Table A.

Table 88: Compound of formula (IV.1), wherein R^{Q}, R^{T}, R^{V} and R^{W} are as defined as in line T14 of Table B, and wherein the definition of the variables M, L, and R^{X} is as defined in a line of Table A.

Table 89: Compound of formula (IV.1), wherein R^{Q}, R^{T}, R^{V} and R^{W} are as defined as in line T15 of Table B, and wherein the definition of the variables M, L, and R^{X} is as defined in a line of Table A.

Table 90: Compound of formula (IV.1), wherein R^{Q}, R^{T}, R^{V} and R^{W} are as defined as in line T16 of Table B, and wherein the definition of the variables M, L, and R^{X} is as defined in a line of Table A.

Table 91: Compound of formula (IV.1), wherein R^{Q}, R^{T}, R^{V} and R^{W} are as defined as in line T17 of Table B, and wherein the definition of the variables M, L, and R^{X} is as defined in a line of Table A.

Table 92: Compound of formula (IV.1), wherein R^{Q}, R^{T}, R^{V} and R^{W} are as defined as in line T18 of Table B, and wherein the definition of the variables M, L, and R^{X} is as defined in a line of Table A.

Table 93: Compound of formula (IV.1), wherein R^{Q}, R^{T}, R^{V} and R^{W} are as defined as in line T19 of Table B, and wherein the definition of the variables M, L, and R^{X} is as defined in a line of Table A.

Table 94: Compound of formula (IV.1), wherein R^{Q}, R^{T}, R^{V} and R^{W} are as defined as in line T20 of Table B, and wherein the definition of the variables M, L, and R^{X} is as defined in a line of Table A.

Table 95: Compound of formula (IV.1), wherein R^{Q}, R^{T}, R^{V} and R^{W} are as defined as in line T21 of Table B, and wherein the definition of the variables M, L, and R^{X} is as defined in a line of Table A.

Table 96: Compound of formula (IV.1), wherein R^{Q}, R^{T}, R^{V} and R^{W} are as defined as in line T22 of Table B, and wherein the definition of the variables M, L, and R^{X} is as defined in a line of Table A.

Table 97: Compound of formula (IV.1), wherein R^{Q}, R^{T}, R^{V} and R^{W} are as defined as in line T23 of Table B, and wherein the definition of the variables M, L, and R^{X} is as defined in a line of Table A.

Table 98: Compound of formula (IV.1), wherein R^{Q}, R^{T}, R^{V} and R^{W} are as defined as in line T24 of Table B, and wherein the definition of the variables M, L, and R^{X} is as defined in a line of Table A.

Table 99: Compound of formula (IV.1), wherein R^{Q}, R^{T}, R^{V} and R^{W} are as defined as in line T25 of Table B, and wherein the definition of the variables M, L, and R^{X} is as defined in a line of Table A.

Table 100: Compound of formula (IV.1), wherein R^{Q}, R^{T}, R^{V} and R^{W} are as defined as in line T26 of Table B, and wherein the definition of the variables M, L, and R^{X} is as defined in a line of Table A.

Table 101: Compound of formula (IV.1), wherein R^{Q}, R^{T}, R^{V} and R^{W} are as defined as in line T27 of Table B, and wherein the definition of the variables M, L, and R^{X} is as defined in a line of Table A.

Table 102: Compound of formula (IV.1), wherein R^{Q}, R^{T}, R^{V} and R^{W} are as defined as in line T28 of Table B, and wherein the definition of the variables M, L, and R^{X} is as defined in a line of Table A.

Table 103: Compound of formula (IV.1), wherein R^{Q}, R^{T}, R^{V} and R^{W} are as defined as in line T29 of Table B, and wherein the definition of the variables M, L, and R^{X} is as defined in a line of Table A.

Table 104: Compound of formula (IV.1), wherein R^{Q}, R^{T}, R^{V} and R^{W} are as defined as in line T30 of Table B, and wherein the definition of the variables M, L, and R^{X} is as defined in a line of Table A.

Table 105: Compound of formula (IV.1), wherein R^{Q}, R^{T}, R^{V} and R^{W} are as defined as in line T31 of Table B, and wherein the definition of the variables M, L, and R^{X} is as defined in a line of Table A.

Table 106: Compound of formula (IV.1), wherein R^{Q}, R^{T}, R^{V} and R^{W} are as defined as in line T32 of Table B, and wherein the definition of the variables M, L, and R^{X} is as defined in a line of Table A.

Table 107: Compound of formula (IV.1), wherein R^{Q}, R^{T}, R^{V} and R^{W} are as defined as in line T33 of Table B, and wherein the definition of the variables M, L, and R^{X} is as defined in a line of Table A.

Table 108: Compound of formula (IV.1), wherein R^{Q}, R^{T}, R^{V} and R^{W} are as defined as in line T34 of Table B, and wherein the definition of the variables M, L, and R^{X} is as defined in a line of Table A.

Table 109: Compound of formula (IV.1), wherein R^{Q}, R^{T}, R^{V} and R^{W} are as defined as in line T35 of Table B, and wherein the definition of the variables M, L, and R^{X} is as defined in a line of Table A.

Table 110: Compound of formula (IV.1), wherein R^{Q}, R^{T}, R^{V} and R^{W} are as defined as in line T36 of Table B, and wherein the definition of the variables M, L, and R^{X} is as defined in a line of Table A.

Table 111: Compound of formula (IV.1), wherein R^{Q}, R^{T}, R^{V} and R^{W} are as defined as in line T37 of Table B, and wherein the definition of the variables M, L, and R^{X} is as defined in a line of Table A.

In one embodiment, the compounds of formula (I) are compounds of formula (II), (III), or (IV) wherein
R^{E}, R^{Q}, R^{T}, R^{V} and R^{W} are independently H; or
   C₁-C₃-alkyl, C₁-C₃-alkoxy, which groups are unsubstituted or halogenated;
each R^{L1}, R^{L2}, R^{M1}, R^{M2} is independently H; C₁-C₃-alkyl, C₁-C₃-haloalkyl; or R^{L1} and R^{L2}, or R^{M1}
   and R^{M2} form together with the carbon atom they are bonded to a group C═O;
each R^{X} is independently halogen;
C₁-C₃-alkyl, C₁-C₃-alkoxy, which groups are unsubstituted or substituted with CN or halogen (such as 1-cyanoisopropyl);
NR³C(═O)R⁹, C(R^{10a})═N—O(R^{10b});
phenyl, which is unsubstituted or halogenated;
a 5- or 6-membered saturated, partially unsaturated, or fully unsaturated heterocyclic ring, wherein said heterocyclic ring comprises one or more, same or different heteroatoms O, N, or S, and is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said N- and S-atoms are independently oxidized, or non-oxidized; or
a group of formula (S),
wherein each R^{S1}, R^{S2} is independently selected from C₁-C₃-alkyl, which groups are unsubstituted, or halogenated;
or two substituents R^{S1}, R^{S2} form, together with the sulfur atom to which they are bound, a 6-membered saturated, partially unsaturated, or fully unsaturated heterocycle, which heterocycle is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said heterocycle comprises no, one or more, same or different heteroatoms O, N, or S in addition to the sulfur atom to which R^{S1} and R^{S2} are bound to.
R^{Y} is C₁-C₃-alkyl, which is unhalogenated or halogenated.
X is phenyl, or a 5- or 6-membered heteroaryl;
Y is SR^{Y1}, SOR^{Y1}, or SO₂R^{Y1}
n is 0 or 1.

In another embodiment, the compounds of formula (I) are compounds of formula (II), (III), or (IV) wherein
R^{E}, R^{Q}, R^{T}, R^{V} and R^{W} are independently H; or
   C₁-C₃-alkyl, C₁-C₃-alkoxy, which groups are unsubstituted or halogenated;
each R^{L1}, R^{L2}, R^{M1}, R^{M2} is independently H; C₁-C₃-alkyl, C₁-C₃-haloalkyl; or R^{L1} and R^{L2}, or R^{M1} and R^{M2} form together with the carbon atom they are bonded to a group C═O;
each R^{X} is independently halogen;
C₁-C₃-alkyl, C₁-C₃-alkoxy, which groups are unsubstituted or substituted with CN or halogen (such as 1-cyanoisopropyl);
NR³C(═O)R⁹, C(R^{10a})═N—OR^{10b});
phenyl, which is unsubstituted or halogenated;
a 5- or 6-membered saturated, partially unsaturated, or fully unsaturated heterocyclic ring, wherein said heterocyclic ring comprises one or more, same or different heteroatoms O, N, or S, and is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said N- and S-atoms are independently oxidized, or non-oxidized; or
a group of formula (S),
wherein each R^{S1}, R^{S2} is independently selected from C₁-C₃-alkyl, which groups are unsubstituted, or halogenated;
or two substituents R^{S1}, R^{S2} form, together with the sulfur atom to which they are bound, a 6-membered saturated, partially unsaturated, or fully unsaturated heterocycle, which heterocycle is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said heterocycle comprises no, one or more, same or different heteroatoms O, N, or S in addition to the sulfur atom to which R^{S1} and R^{S2} are bound to.
R⁹ is C₁-C₃-alkyl or C₁-C₃-haloalkyl;
R^{10a} is H, CN, C₁-C₃-alkyl;
R^{19b} is H, C₁-C₃-haloalkyl.
R^{Y1} is C₁-C₃-alkyl, which is unhalogenated or halogenated.
X is phenyl, or 2-pyridyl;
Y is S, SO, or SO₂
n is 0 or 1.

In another embodiment, the compounds of formula (I) are compounds of formula (II), (III), or (IV) wherein
R^{E}, R^{Q}, R^{T}, R^{V} and R^{W} are independently H; or
   C₁-C₃-alkyl, C₁-C₃-alkoxy, which groups are unsubstituted or halogenated;
each R^{L1}, R^{L2}, R^{M1}, R^{M2} is independently H; C₁-C₃-alkyl, C₁-C₃-haloalkyl; or R^{L1} and R^{L2}, or R^{M1} and R^{M2} form together with the carbon atom they are bonded to a group C═O;
each R^{X} is independently halogen;
C₁-C₆-cycloalkyl, which is unsubstituted or substituted with halogen;
phenyl, which is unsubstituted or halogenated;
a 5- or 6-membered saturated, partially unsaturated, or fully unsaturated heterocyclic ring, wherein said heterocyclic ring comprises one or more, same or different heteroatoms O, N, or S, and is unsubstituted, or substituted with one or more, same or different substituents selected from halogen, C₁-C₃-alkyl, C₁-C₃-alkoxy, C₁-C₃-haloalkyl, and C₁-C₃-haloalkoxy, or wherein the ring contains a group C═O that is connected to the other ring members via the carbon atom; or
a group of formula (S),
wherein each R^{S1}, R^{S2} is independently selected from C₁-C₃-alkyl, which groups are unsubstituted, or halogenated;
R⁹ is C₁-C₃-alkyl or C₁-C₃-haloalkyl;
R^{Y1} is C₁-C₃-alkyl, which is unhalogenated or halogenated.
X is phenyl, or 2-pyridyl;
Y is SR^{Y1}, SOR^{Y1}, or SO₂R^{Y1};
n is 0 or 1.

In another embodiment, the compounds of formula (I) are compounds of formula (II), or (IV) wherein
R^{E}, R^{Q}, R^{T}, R^{V} and R^{W} are independently H; or
   C₁-C₃-alkyl, C₁-C₃-alkoxy, which groups are unsubstituted or halogenated;
each R^{L1}, R^{L2}, R^{M1}, R^{M2} is independently H;
each R^{X} is independently halogen;
C₁-C₆-cycloalkyl, which is unsubstituted or substituted with halogen;
phenyl, which is unsubstituted or halogenated;
a 5- or 6-membered saturated, partially unsaturated, or fully unsaturated heterocyclic ring, wherein said heterocyclic ring comprises one or more, same or different heteroatoms O, N, or S, and is unsubstituted, or substituted with one or more, same or different substituents selected from halogen, C₁-C₃-alkyl, C₁-C₃-alkoxy, C₁-C₃-haloalkyl, and C₁-C₃-haloalkoxy, or wherein the ring contains a group C═O that is connected to the other ring members via the carbon atom; or a group of formula (S),
wherein each R^{S1}, R^{S2} is independently selected from C₁-C₃-alkyl, which groups are unsubstituted, or halogenated;
R^{Y1} is C₁-C₃-alkyl, which is unhalogenated or halogenated.
X is phenyl, or 2-pyridyl;
Y is SR^{Y1}, SOR^{Y1}, or SO₂R^{Y1}
n is 0 or 1.

In another embodiment, the compounds of formula (I) are compounds of formula (II), or (IV) wherein
R^{E}, R^{Q}, R^{T}, R^{V} and R^{W} are independently H; or
   C₁-C₃-alkyl, C₁-C₃-alkoxy, which groups are unsubstituted or halogenated;
each R^{L1}, R^{L2}, R^{M1}, R^{M2} is independently H;
each R^{X} is independently halogen;
phenyl, which is unsubstituted or halogenated;
a group of formula (S),
wherein each R^{S1}, R^{S2} is independently selected from C₁-C₃-alkyl;
R^{Y1} is C₁-C₃-alkyl, which is unhalogenated or halogenated.
X is 2-pyridyl;
Y is SR^{Y1}, SOR^{Y1}, or SO₂R^{Y1};
n is 0 or 1.

The term “compound(s) of the invention” refers to compound(s) of formula (I), or “compound(s) (I)”, and includes their salts, tautomers, stereoisomers, and N-oxides.

The invention also relates to agrochemical compositions comprising an auxiliary and at least one compound (I).

An agrochemical composition comprises a pesticidally effective amount of a compound (I).

The compounds I can be converted into customary types of agro-chemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials e.g. seeds (e.g. GF). These and further compositions types are defined in the “Catalogue of pesticide formulation types and international coding system”, Technical Monograph No. 2, 6th Ed. May 2008, CropLife International. The compositions are prepared in a known manner, e.g. described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents. Suitable solid carriers or fillers are mineral earths.

Suitable surfactants are surface-active compounds, e.g. anionic, cationic, nonionic, and amphoteric surfactants, block polymers, polyelectrolytes. Such surfactants can be used as emusifier, dispersant, solubilizer, wetter, penetration enhancer, protective colloid, or adjuvant. Surfactants are listed in McCutcheon's, Vol. 1: Emulsifiers & Detergents, McCutcheon's Directories, Glen Rock, USA, 2008 (International or North American Ed.). Suitable anionic surfactants are alkali, alkaline earth, or ammonium salts of sulfonates, sulfates, phosphates, carboxylates. Suitable nonionic surfactants are alkoxylates, N-subsituted fatty acid amides, amine oxides, esters, sugar-based surfactants, polymeric surfactants. Suitable cationic surfactants are qua-ternary surfactants.

The agrochemical compositions generally comprise between 0.01 and 95%, preferably between 0.1 and 90%, and most preferably between 0.5 and 75%, by weight of active substance. The active substances are employed in a purity of from 90% to 100%, preferably from 95% to 100%.

Various types of oils, wetters, adjuvants, or fertilizer may be added to the active substances or the compositions comprising them as premix or, if appropriate not until immediately prior to use (tank mix). These agents can be admixed with the compositions according to the invention in a weight ratio of 1:100 to 100:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, of the ready-to-use spray liquor are applied per hectare of agricultural useful area.

The compounds I are suitable for use in protecting crops, plants, plant propagation materials, e.g. seeds, or soil or water, in which the plants are growing, from attack or infestation by animal pests. Therefore, the invention also relates to a plant protection method, which comprises contacting crops, plants, plant propagation materials, e.g. seeds, or soil or water, in which the plants are growing, to be protected from attack or infestation by animal pests, with a pesticidally effective amount of a compound (I).

The compounds I are also suitable for use in combating or controlling animal pests. There-fore, the invention also relates to a method of combating or controlling animal pests, which comprises contacting the animal pests, their habitat, breeding ground, or food supply, or the crops, plants, plant propagation materials, e.g. seeds, or soil, or the area, material or environment in which the animal pests are growing or may grow, with a pesticidally effective amount of a compound (I).

The compounds I are effective through both contact and ingestion to any and all developmental stages, such as egg, larva, pupa, and adult.

The compounds I can be applied as such or in form of compositions comprising them.

The application can be carried out both before and after the infestation of the crops, plants, plant propagation materials by the pests.

The term “contacting” includes both direct contact (applying the compounds/compositions directly on the animal pest or plant) and indirect contact (applying the compounds/compositions to the locus).

The term “animal pest” includes arthropods, gastropods, and nematodes. Preferred animal pests according to the invention are arthropods, preferably insects and arachnids, in particular insects.

The term “plant” includes cereals, e.g. durum and other wheat, rye, barley, triticale, oats, rice, or maize (fodder maize and sugar maize/sweet and field corn); beet, e.g. sugar beet, or fodder beet; fruits, e.g. pomes, stone fruits, or soft fruits, e.g. apples, pears, plums, peaches, nectarines, almonds, cherries, papayas, strawberries, raspberries, blackberries or gooseberries; leguminous plants, e.g. beans, lentils, peas, alfalfa, or soybeans; oil plants, e.g. rapeseed (oilseed rape), turnip rape, mustard, olives, sunflowers, coconut, cocoa beans, castor oil plants, oil palms, ground nuts, or soybeans; cucurbits, e.g. squashes, pumpkins, cucumber or melons; fiber plants, e.g. cotton, flax, hemp, or jute; citrus fruit, e.g. oranges, lemons, grape-fruits or mandarins; vegetables, e.g. eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, e.g. avocados, cinnamon, or camphor; energy and raw material plants, e.g. corn, soybean, rapeseed, sugar cane or oil palm; tobacco; nuts, e.g. walnuts; pistachios; coffee; tea; bananas; vines; hop; sweet leaf (Stevia); natural rubber plants or ornamental and forestry plants, shrubs, broad-leaved trees or evergreens, eucalyptus; turf; lawn; grass. Preferred plants include potatoes sugar beets, tobacco, wheat, rye, barley, oats, rice, corn, cotton, soybeans, rapeseed, legumes, sunflowers, coffee, or sugar cane; fruits; vines; ornamentals; or vegetables, e.g. cucumbers, tomatoes, beans or squashes.

The term “seed” embraces seeds and plant propagules including true seeds, seed pieces, suckers, corms, bulbs, fruit, tubers, grains, cuttings, cut shoots, and means preferably true seeds.

“Pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions e.g. desired pesticidal effect and duration, weather, target species, locus, mode of application.

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare.

The compounds I are also suitable for use against non-crop insect pests. For use against said non-crop pests, compounds I can be used as bait composition, gel, general insect spray, aerosol, as ultra-low volume application and bed net (impregnated or surface applied).

The term “non-crop insect pest” refers to pests, which are particularly relevant for non-crop targets, e.g. ants, termites, wasps, flies, ticks, mosquitoes, bed bugs, crickets, or cockroaches, such as: Aedes aegypti, Musca domestica, Tribolium spp.; termites such as Reticulitermes flavipes, Coptotermes formosanus; roaches such as Blatella germanica, Periplaneta Americana; ants such as Solenopsis invicta, Linepithema humile, and Camponotus pennsylvanicus.

The bait can be a liquid, a solid or a semisolid preparation (e.g. a gel). For use in bait compositions, the typical content of active ingredient is from 0.001 wt % to 15 wt %, desirably from 0.001 wt % to 5 wt % of active compound.

The compounds I and its compositions can be used for protecting wooden materials such as trees, board fences, sleepers, frames, artistic artifacts, etc. and buildings, but also construction materials, furniture, leathers, fibers, vinyl articles, electric wires and cables etc. from ants, termites and/or wood or textile destroying beetles, and for controlling ants and termites from doing harm to crops or human beings (e.g. when the pests invade into houses and public facilities or nest in yards, orchards or parks).

Customary application rates in the protection of materials are, e.g., from 0.001 g to 2000 g or from 0.01 g to 1000 g of active compound per m² treated material, desirably from 0.1 g to 50 g per m². Insecticidal compositions for use in the impregnation of materials typically contain from 0.001 to 95 wt %, preferably from 0.1 to 45 wt %, and more preferably from 1 to 25 wt % of at least one repellent and/or insecticide.

### Pests

The compounds of the invention are especially suitable for efficiently combating animal pests e.g. arthropods, and nematodes including:
insects from the sub-order of Auchenorrhyncha, e.g. Amrasca biguttula, Empoasca spp., Nephotettix virescens, Sogatella furcifera, Mahanarva spp., Laodelphax striatellus, Nilaparvata lugens, Diaphorina citri;
Lepidoptera, e.g. Helicoverpa spp., Heliothis virescens, Lobesia botrana, Ostrinia nubilalis, Plutella xylostella, Pseudoplusia includens, Scirpophaga incertulas, Spodoptera spp., Trichoplusia ni, Tuta absoluta, Cnaphalocrocis medialis, Cydia pomonella, Chilo suppressalis, Anticarsia gemmatalis, Agrotis ipsilon, Chrysodeixis includens;
True bugs, e.g. Lygus spp., Stink bugs such as Euschistus spp., Halyomorpha halys, Nezara viridula, Piezodorus guildinii, Dichelops furcatus;
Thrips, e.g. Frankliniella spp., Thrips spp., Dichromothrips corbettii;
Aphids, e.g. Acyrthosiphon pisum, Aphis spp., Myzus persicae, Rhopalosiphum spp., Schizaphis graminum, Megoura viciae;
Whiteflies, e.g. Trialeurodes vaporariorum, Bemisia spp.;
Coleoptera, e.g. Phyllotreta spp., Melanotus spp., Meligethes aeneus, Leptinotarsa decimlineata, Ceutorhynchus spp., Diabrotica spp., Anthonomus grandis, Atomaria linearia, Agriotes spp., Epilachna spp., Phyllotreta spp (e.g. P. vittula), Popilla spp. (e.g. Popilla japonica);
Flies, e.g. Delia spp., Ceratitis capitate, Bactrocera spp., Liriomyza spp.;
Coccoidea, e.g. Aonidiella aurantia, Ferrisia virgate;
Anthropods of class Arachnida (Mites), e.g. Penthaleus major, Tetranychus spp.;
Nematodes, e.g. Heterodera glycines, Meloidogyne spp., Pratylenchus spp., Caenorhabditis elegans.

### Animal Health

The compounds I are suitable for use in treating or protecting animals against infestation or infection by parasites. Therefore, the invention also relates to the use of a compound of the invention for the manufacture of a medicament for the treatment or protection of animals against infestation or infection by parasites. Furthermore, the invention relates to a method of treating or protecting animals against infestation and infection by parasites, which comprises orally, topically or parenterally administering or applying to the animals a parasiticidally effective amount of a compound (I).

The invention also relates to the non-therapeutic use of compounds of the invention for treating or protecting animals against infestation and infection by parasites. Moreover, the invention relates to a non-therapeutic method of treating or protecting animals against infestation and infection by parasites, which comprises applying to a locus a parasiticidally effective amount of a compound (I).

The compounds of the invention are further suitable for use in combating or controlling parasites in and on animals. Furthermore, the invention relates to a method of combating or controlling parasites in and on animals, which comprises contacting the parasites with a parasitically effective amount of a compound (I).

The invention also relates to the non-therapeutic use of compounds I for controlling or combating parasites. Moreover, the invention relates to a non-therapeutic method of combating or controlling parasites, which comprises applying to a locus a parasiticidally effective amount of a compound (I).

The compounds I can be effective through both contact (via soil, glass, wall, bed net, carpet, blankets or animal parts) and ingestion (e.g. baits). Furthermore, the compounds I can be applied to any and all developmental stages.

The compounds I can be applied as such or in form of compositions comprising them.

The term “locus” means the habitat, food supply, breeding ground, area, material or environment in which a parasite is growing or may grow outside of the animal.

As used herein, the term “parasites” includes endo- and ectoparasites. In some embodiments of the invention, endoparasites can be preferred. In other embodiments, ectoparasites can be preferred. Infestations in warm-blooded animals and fish include lice, biting lice, ticks, nasal bots, keds, biting flies, muscoid flies, flies, myiasitic fly larvae, chiggers, gnats, mosquitoes and fleas.

The compounds of the invention are especially useful for combating the following parasites: Cimex lectularius, Rhipicephalus sanguineus, and Ctenocephalides fells.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

The compounds I may be applied in total amounts of 0.5 mg/kg to 100 mg/kg per day, preferably 1 mg/kg to 50 mg/kg per day.

For oral administration to warm-blooded animals, the compounds I may be formulated as animal feeds, animal feed premixes, animal feed concentrates, pills, solutions, pastes, suspensions, drenches, gels, tablets, boluses and capsules. For oral administration, the dosage form chosen should provide the animal with 0.01 mg/kg to 100 mg/kg of animal body weight per day of the compounds I, preferably with 0.5 mg/kg to 100 mg/kg of animal body weight per day.

Alternatively, the compounds I may be administered to animals parenterally, e.g., by intraruminal, intramuscular, intravenous or subcutaneous injection. The compounds I may be dispersed or dissolved in a physiologically acceptable carrier for subcutaneous injection. Alternatively, the compounds I may be formulated into an implant for subcutaneous administration. In addition the compounds I may be transdermally administered to animals. For parenteral administration, the dosage form chosen should provide the animal with 0.01 mg/kg to 100 mg/kg of animal body weight per day of the compounds I.

The compounds I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds I. In addition, the compounds I may be formulated as ear tags for animals, particularly quadrupeds e.g. cattle and sheep.

Oral solutions are administered directly.

Solutions for use on the skin are trickled on, spread on, rubbed in, sprinkled on or sprayed on.

Gels are applied to or spread on the skin or introduced into body cavities.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures.

Emulsions can be administered orally, dermally or as injections.

Suspensions can be administered orally or topically/dermally.

Semi-solid preparations can be administered orally or topically/dermally.

For the production of solid preparations, the active compound is mixed with suitable excipients, if appropriate with addition of auxiliaries, and brought into the desired form.

The compositions which can be used in the invention can comprise generally from about 0.001 to 95% of the compound I.

Ready-to-use preparations contain the compounds acting against parasites, preferably ectoparasites, in concentrations of 10 ppm to 80% by weight, preferably from 0.1 to 65% by weight, more preferably from 1 to 50% by weight, most preferably from 5 to 40% by weight.

Preparations which are diluted before use contain the compounds acting against ectoparasites in concentrations of 0.5 to 90% by weight, preferably of 1 to 50% by weight.

Furthermore, the preparations comprise the compounds of formula (I) against endoparasites in concentrations of 10 ppm to 2% by weight, preferably of 0.05 to 0.9% by weight, very particularly preferably of 0.005 to 0.25% by weight.

Solid formulations which release compounds of the invention may be applied in total amounts of 10 mg/kg to 300 mg/kg, preferably 20 mg/kg to 200 mg/kg, most preferably 25 mg/kg to 160 mg/kg body weight of the treated animal in the course of three weeks.

The following examples illustrate the invention.

### A. PREPARATION OF COMPOUNDS

Materials: Unless otherwise noted, reagents and solvents were purchased at highest commercial quality and used without further purification. Dry tetrahydrofuran (THF), ethylacetate (EtOAc), dimethylsulfoxide (DMSO), acetone, ethanol (EtOH), benzene, dimethylformamide (DMF), diisopropylethylamine (DI PEA), hexafluorophosphate azabenzotriazole tetramethyl uronium (HATU), pyridine, and CH₂Cl₂ were purchased from commercial providers.

All reactions were monitored by thin-layer chromatography (TLC) using Merck silica gel 60 F₂54 pre-coated plates (0.25 mm). Flash chromatography was carried out with Kanto Chemical silica gel (Kanto Chemical, silica gel 60N, spherical neutral, 0.040-0.050 mm, Cat.-No. 37563-84). ¹H NMR spectra were recorded on JEOL JNM-ECA-500 (500 MHz). Chemical shifts are expressed in ppm downfield from the internal solvent peaks for acetone-d₆ (¹H; δ=2.05 ppm) and CD₃OD (¹H; δ=3.30 ppm), and J values are given in Hertz. The following abbreviations were used to explain the multiplicities: s=singlet, d=doublet, t=triplet, q=quartet, dd=double doublet, dt=double triplet, m=multiplet, br=broad. High-resolution mass spectra were measured on a JEOL JMS-T100LP.

### Characterization

Method A: The compounds were characterized by coupled High Performance Liquid Chromatography with mass spectrometry (HPLC/MS). UHPLC-MS on Shimadzu Nexera UHPLC & Shimadzu LCMS 20-20 ESI. Analytical UHPLC column: Phenomenex Kinetex 1,7 μm XB-C18 100A; 50×2.1 mm; mobile phase: A: water+0.1% TFA; B: acetonitrile; gradient: 5-100% B in 1.50 minutes; 100% B 0.20 min; flow: 0.8-1.0 mL/min in 1.50 minutes at 60° C. MS-method: ESI positive; mass range (m/z) 100-700.

Method B: Agilent 1260 HPLC MSD: 6125B single quadrupole MSD Column: Luna C18, 2.0*50 mm, 5 μm Column Temp: 40 Mobile Phase:A: 0.04% TFA in H₂O Mobile

Phase: B: 0.02% TFA in ACN Flow Rate: 1 ml/min

Method C: Shimadzu LC-20AB. Analytical UHPLC column: C-18, 50 mm, 2.1 mm, 5 micron; mobile phase: A: 0.04% TFA in Water. B: 0.02% TFA in Acetonitrile. Flow Rate: 1.2 mL/min, Injection Vol: 0.3 μL; Gradient: 10% B to 80% B in 4 min, 10% B for 30 sec. Run time: 4.5 min at 40° C.

### Synthesis Example 1: Preparation of 2-[3-ethylsulfonyl-5-(4-fluorophenyl)-2-pyridyl]-6-(trifluoromethyl)-4H-imidazo[2,1-c][1,4]benzoxazine (Compound I.4)

### Step 1: Preparation of 1-[3-ethylsulfonyl-5-(4-fluorophenyl)-2-pyridyl]ethenone (1)

To the solution of 1-(5-bromo-3-ethylsulfonyl-2-pyridyl)ethenone (17.12 mmol) and (4-fluorophenyl)boronic acid (22.26 mmol) in 1,4-dioxane (50 ml) was added K₂CO₃ (51.37 mmol) in H₂O (10 mL) and Pd(PPh₃)₄ (0.85 mmol). The resulting reactionmixture was stirred at 110° C. for 2 hours. The reachtion mixture was quenched with H₂O and extracted. The combined organic layers were washed, dried and concentrated. The crude concentrate was purified by column chromatography to afford 1-[3-ethylsulfonyl-5-(4-fluorophenyl)-2-pyridyl]ethenone (5 g, 95% yield) as a yellow solid. ¹H NMR (400 MHz, CHLOROFORM-d) δ ppm 8.90 (d, J=2.1 Hz, 1H), 8.42 (d, J=2.1 Hz, 1H), 7.60-7.53 (m, 2H), 7.21-7.14 (m, 2H), 3.56 (q, J=7.4 Hz, 2H), 2.73-2.62 (m, 3H), 1.30 (t, J=7.4 Hz, 3H)

### Step 2: Preparation of 2-bromo-1-[3-ethylsulfonyl-5-(4-fluorophenyl)-2-pyridyl]ethenone

To the solution of 1-[3-ethylsulfonyl-5-(4-fluorophenyl)-2-pyridyl]ethenone (16.3 mmol) in toluene (100 ml) was added HBr (33% in CH₃COOH, 15 ml) dropwise at 0° C. and the resulting reaction mixture was stirred at 25° C. for 15 min. Then, Br 2 (2.8 g, 17.9 mmol) was added dropwise at 25° C. The reaction mixture was stirred at 25° C. for 16 hours. The reaction mixture was then quenched with an aqueous saturated solution of NaHCO₃ and extracted. The combined organic layers were washed, dried and concentrated. The crude product was triturated with 2-methoxy-2-methylpropan afford 2-bromo-1-[3-ethylsulfonyl-5-(4-fluorophenyl)-2-pyridyl]ethenone (5.3 g, 90% yield). ¹H NMR (400 MHz, DMSO-d₆) δ ppm 9.29 (d, J=2.1 Hz, 1H), 8.58 (d, J=2.0 Hz, 1H), 8.05-7.88 (m, 2H), 7.53-7.34 (m, 2H), 4.95 (s, 2H), 3.72-3.54 (m, 2H), 1.33-1.15 (m, 3H)

### Step 3: Preparation of 2-nitro-6-(trifluoromethyl)phenol

To the solution of 2-(trifluoromethyl)phenol (150 mmol) in CH₃COOH (50 ml) was added HNO₃ (8 ml) dropwise at 0° C. The resulting reaction mixture was stirred at 25° C. for 2 hours. The reaction mixture was quenched and extracted. The combined organic layers were washed, dried and concentrated. The resulting crude product was purified by column to afford 2-nitro-6(trifluoromethyl)phenol (25 g, crude) and used in next step directly.

### Step 4: Preparation of ethyl 2-[2-nitro-6-(trifluoromethyl)phenoxy]acetate

To the solution of 2-nitro-6-(trifluoromethyl)phenol (120 mmol) and K₂CO₃ (300 mmol) in CH₃CN (500 ml) was added ethyl 2-bromoacetate (130 mmol) and the resulting reaction mixture was stirred at 80° C. for 2 hours. The reaction mixture was quenched and extracted. The combined organic layers were washed, dried and concentrated to give ethyl 2-[2-nitro-6-(trifluoromethyl)phenoxy]acetate (26 g, crude), which was used in next step directly.

### Step 5: Preparation of 8-(trifluoromethyl)-4H-1,4-benzoxazin-3-one

To a solution of ethyl 2-[2-nitro-6-(trifluoromethyl)phenoxy]acetate (88.7 mmol) in THF (300 ml) was added Pd/C (9.4 g, 8.9 mmol) and the resulting reaction mixture was stirred at 50° C. for 72 hours under H₂ (50 Psi). Then, the reaction mixture was filtered, and the filtrate was concentrated to afford a crude product. The crude product was purified by column to afford 8-(trifluoromethyl)-4H-1,4-benzoxazin-3-one (10 g, 31% yield for 3 steps) as a white solid. ¹H-NMR (400 MHz, CDCl₃) δ ppm 9.09 (br s, 1H), 7.32-7.26 (m, 1H), 7.06 (br d, J=4.5 Hz, 2H), 4.74 (s, 2H)

### Step 6: Preparation of 8-(trifluoromethyl)-4H-1,4-benzoxazine-3-thione

To a solution of 8-(trifluoromethyl)-4H-1,4-benzoxazin-3-one (46 mmol) in toluene (150 ml) was added P₂S₅ (230 mmol) and the mixture was stirred at 110° C. for 32 hours. The reaction mixture was concentrated and the resulting crude product was purified by silica gel column to afford 8-(trifluoromethyl)-4H-1,4-benzoxazine-3-thione (5.7 g, 53% yield). ¹H-NMR (400 MHz, DMSO-d₆) δ ppm 12.96 (br s, 1H), 7.38-7.32 (m, 2H), 7.18-7.14 (m, 1H), 4.98 (s, 2H)

### Step 7: Preparation of 3-methylsulfanyl-8-(trifluoromethyl)-2H-1,4-benzoxazine

To a solution of 8-(trifluoromethyl)-4H-1,4-benzoxazine-3-thione (24.46 mmol) and K₂CO₃ (73.39 mmol) in CH₃CN (50 ml) was added CH₃I (36.69 mmol) and the resulting reaction mixture was stirred at 25° C. for 16 hours. The reaction mixture was quenched and extracted. The combined organic layers were washed, dried and concentrated. The resulting crude product was purified by column to afford 3-methylsulfanyl-8-(trifluoromethyl)-2H-1,4-benzoxazine (4.8 g, 79% yield). ¹H-NMR (400MHz, CDCl₃) δ ppm 7.45 (br t, J=7.6 Hz, 1H), 7.34 (br t, J=7.5 Hz, 1H), 7.05 (q, J=7.7 Hz, 1H), 4.59 (d, J=7.9 Hz, 2H), 2.58 (d, J=7.7 Hz, 3H)

### Step 8: Preparation of 8-(trifluoromethyl)-2H-1,4-benzoxazin-3-amine

To a solution of 3-methylsulfanyl-8-(trifluoromethyl)-2H-1,4-benzoxazine (19.35 mmol) in NH₃/CH₃OH (100 ml) was stirred at 25° C. for 16 hours. LC-MS showed the starting material was consumed completely. The mixture was concentrated and the crude was purified by trituration by methyl-tert-butyl ether (20 ml) to afford 8-(trifluoromethyl)-2H-1,4-benzoxazin-3-amine (3.4 g, 81% yield). ¹H NMR (400 MHz, DMSO-d₆) δ ppm 7.08 (br t, J=7.6 Hz, 2H), 7.00-6.94 (m, 1H), 4.53 (s, 2H)

### Step 9: Preparation of 2-[3-ethylsulfonyl-5-(4-fluorophenyl)-2-pyridyl]-6-(trifluoromethyl)-4H-imidazo[2,1-c][1,4]benzoxazine (Compound I.4)

To a solution of 8-(trifluoromethyl)-2H-1,4-benzoxazin-3-amine (2.3 mmol) and 2-bromo-1-[3-ethylsulfonyl-5-(4-fluorophenyl)-2-pyridyl]ethenone (1.1 g, 2.8 mmol) in (CH₃)₃COH (50 ml) was added 4 Å molecular sieves (500 mg). The resulting reaction mixture was stirred at 110° C. for 48 hours.

Then, the reaction mixture was filtered, and the filtrate was concentrated to afford a crude product. The crude product was purified by HPLC and triturated in CH₃CN to afford 2-[3-ethylsulfonyl-5-(4-fluorophenyl)-2-pyridyl]-6-(trifluoromethyl)-4H-imidazo[2,1-c][1,4]benzoxazine (112.5 mg, 10% yield) as a yellow solid. ¹H NMR (400 MHz, DMSO-d₆) δ ppm 9.20 (d, J=1.3 Hz, 1H), 8.65 (s, 1H), 8.53 (d, J=1.5 Hz, 1H), 8.23 (d, J=8.3 Hz, 1H), 7.93 (dd, J=5.7, 8.1 Hz, 2H), 7.60 (d, J=7.9 Hz, 1H), 7.46-7.33 (m, 3H), 5.57 (s, 2H), 4.13 (q, J=7.3 Hz, 2H), 1.27 (t, J=7.5 Hz, 3H) LC/MS retention time: 1,659 min, m/z=504 (M+H⁺)

### Synthesis Example 2: Synthesis of 2-[3-ethylsulfonyl-5-(4-fluorophenyl)-2-pyridyl]-6-(trifluoromethyl)-5H-imidazo[1,2-a][3,1]benzoxazine (Compound I.3)

### Step 1: Preparation of [2-amino-6-(trifluoromethyl)phenyl]methanol

To the solution of [2-carboxy-3-(trifluoromethyl)phenyl]ammonium; chloride (25 g, 100 mmol) in THF (300 ml) was added BH₃·(CH₃)₂S (10 M, 26 ml, 260 mmol) at 0° C. and the resulting reaction mixture was stirred at 25° C. for 16 hours. After completion of the reaction, CH₃OH (100 ml) was added dropwise to the mixture at 0° C. The reaction mixture was then concentrated and the resulting crude product was added to H₂O and extracted. The combined organic layers were washed, dried, and concentrated to afford [2-amino-6-(trifluoromethyl)phenyl]methanol (10 g, crude), which was used in the next step directly.

### Step 2: (E)-[5-(trifluoromethyl)-1,4-dihydro-3,1-benzoxazin-2-ylidene]cyanamide

A solution of [2-amino-6-(trifluoromethyl)phenyl]methanol (4 g, 21 mmol) and diphenoxymethylcyanamide in (CH₃)₂CHOH (100 ml) was stirred at 80° C. for 16 hours. After completion of the reaction, the reaction mixture was concentrated and purification by HPLC to afford (E)-[5-(trifluoromethyl)-1,4-dihydro-3,1-benzoxazin-2-ylidene]cyanamide (2 g, 40% yield).

### Step 3: Preparation of 5-(trifluoromethyl)-4H-3,1-benzoxazin-2-amine

A solution of (E)-[5-(trifluoromethyl)-1,4-dihydro-3,1-benzoxazin-2-ylidene]cyanamide (4.1 mmol) in NH₄OH (10 ml) was stirred at 90° C. for 8 hours. After completion of the reaction, the reaction mixture was quenched with H₂O and extracted. The combined organic layers were washed, dried and concentrated to afford 5-(trifluoromethyl)-4H-3,1-benzoxazin-2-amine (600 mg, crude), which was used in the next step directly.

### Step 4: Preparation of 2-[3-ethylsulfonyl-5-(4-fluorophenyl)-2-pyridyl]-6-(trifluoromethyl)-5H-imidazo[1,2-a][3,1]benzoxazine (Compound I.3)

To a solution of 5-(trifluoromethyl)-4H-3,1-benzoxazin-2-amine (1.4 mmol) and 2-bromo-1-[3-ethylsulfonyl-5-(4-fluorophenyl)-2-pyridyl]ethenone (1.4 mmol) in (CH₃)₃COH (50 ml) was added 4 Å molecular sieves (300 mg) and the resulting reaction mixture was stirred at 110° C. for 48 hours. After completion of the reaction, thre reaction mixture was filtered, and the filtrate was concentrated to afford a crude product. The crude product was purified by HPLC and triturated by CH₃CN to afford 2-[3-ethylsulfonyl-5-(4-fluorophenyl)-2-pyridyl]-6-(trifluoromethyl)-5H-imidazo[1,2-a][3,1]benzoxazine (73 mg, 3% yield for 4 steps). ¹H-NMR (400 MHz, DMSO-d₆) δ ppm 9.17 (s, 1H), 8.51 (s, 1H), 8.41 (s, 1H), 8.15 (br d, J=7.4 Hz, 1H), 7.96-7.88 (m, 2H), 7.78-7.68 (m, 2H), 7.41 (br t, J=8.6 Hz, 2H), 5.67 (s, 2H), 4.13 (q, J=7.3 Hz, 2H), 1.26 (br t, J=7.3 Hz, 3H) LC/MS retention time: 1,646 min, m/z=504 (M+H⁺).

### Synthesis Example 3: Synthesis of 2-[3-ethylsulfonyl-5-(4-fluorophenyl)-2-pyridyl]-6-(trifluoromethyl)-4H-imidazo[2,1-c[]1,4]benzothiazine (Compound I.2)

### Step 1: Preparation of sodium;2-nitro-6-(trifluoromethyl)benzenethiolate

To the solution of 2-fluoro-1-nitro-3-(trifluoromethyl)benzene (47.8 mmol) in DMF (100 ml) was added Na₂S (95.6 mmol) slowly at 0° C. and the mixture was stirred at 0° C. for 2 hours. After completion of the reaction, sodium;2-nitro-6-(trifluoromethyl)benzenethiolate was used for the next step directly as DMF solution.

### Step 2: Preparation of 2-[2-nitro-6-(trifluoromethyl)phenyl]sulfanylacetonitrile

To a solution of sodium;2-nitro-6-(trifluoromethyl)benzenethiolate (23.9 mmol) in DMF (20 ml) was added 2-chloroacetonitrile (71.8 mmol) dropwise at 0° C. The resulting reaction mixture was stirred at 25° C. for 2 hours. After completion of the reaction, the reaction was quenched with H₂O and the resulting mixture was extracted. The combined organic layers were washed, dried and concentrated to afford a crude product. The crude product was purified by column to afford 2-[2-nitro-6-(trifluoromethyl)phenyl]sulfanylacetonitrile (13 g, crude), which was used in the next step directly. ¹H-NMR (400 MHz, CDCl₃) δ ppm 8.05 (d, J=8.2 Hz, 1H), 7.92 (d, J=8.0 Hz, 1H), 7.83-7.77 (m, 1H), 3.78 (s, 2H)

### Step 3: Preparation of 8-(trifluoromethyl)-2H-1,4-benzothiazin-3-amine

To a solution of 2-[2-nitro-6-(trifluoromethyl)phenyl]sulfanylacetonitrile (7.6 mmol) in CH₃COOCH₂CH₃ (120 ml) was added Pd/C (81 mg, 0.76 mmol) and the resulting reaction mixture was stirred at 25° C. for 4 hours under H₂ (50 Psi). After completion of the reaction, the reaction mixture was filtered, and the filtrate was concentrated to afford 8-(trifluoromethyl)-2H-1,4-benzothiazin-3-amine (1.1 g, 62.6% yield). ¹H-NMR (400 MHz, CDCl₃) δ ppm 7.34-7.29 (m, 1H), 7.24-7.17 (m, 2H), 3.11 (s, 2H).

### Step 4: Preparation of 2-[3-ethylsulfonyl-5-(4-fluorophenyl)-2-pyridyl]-6-(trifluoromethyl)-4H-imidazo[2,1-d][1,4]benzothiazine (Compound I.2)

To a solution of 8-(trifluoromethyl)-2H-1,4-benzothiazin-3-amine (1.5 mmol) and 2-bromo-1-[3-ethylsulfonyl-5-(4-fluorophenyl)-2-pyridyl]ethenone (1.5 mmol) in (CH₃)₃COH (50 ml) was added 4 Å molecular sieves (350 mg) and the resulting reaction mixture was stirred at 110° C. for 48 hours. After completion of the reaction, the mixture was filtered and the filtrate was concentrated to afford a crude product. The crude product was purified by trituration in CH₃CN to afford 2-[3-ethylsulfonyl-5-(4-fluorophenyl)-2-pyridyl]-6-(trifluoromethyl)-4H-imidazo[2,1-c][1,4]benzothiazine (125 mg, 16% yield). ¹H NMR (400 MHz, DMSO-d₆) δ ppm 9.20 (d, J=2.2 Hz, 1H), 8.54 (s, 2H), 8.20 (d, J=8.6 Hz, 1H), 7.97-7.89 (m, 2H), 7.76 (d, J=8.3 Hz, 1H), 7.65-7.56 (m, 1H), 7.42 (t, J=8.9 Hz, 2H), 4.36 (s, 2H), 4.19 (q, J=7.1 Hz, 2H), 1.28 (t, J=7.3 Hz, 3H) LC/MS retention time: 1,668 min, m/z=520 (M+H⁺)

### Synthesis Example 4: Synthesis of 2-[3-ethylsulfonyl-5-(4-fluorophenyl)-2-pyridyl]-6-(trifluoromethyl)-5H-imidazo[1,2-a][3,1]benzothiazine (Compound I.1)

### Step 1: Preparation of [2-(chloromethyl)-3-(trifluoromethyl)phenyl]ammonium;hydrochloride

A solution of [2-amino-6-(trifluoromethyl)phenyl]methanol (20.9 mmol) in concentrated, aqueous solution of HCl (12 N, 100 ml) was stirred at 100° C. for 16 hours. After completion of the reaction, the reaction mixture was concentrated and the resulting crude product was purified by trituration in CH₃COOCH₂CH₃ to afford [2-(chloromethyl)-3-(trifluoromethyl)-phenyl]ammonium;hydrochloride (3.8 g, crude), which was used in the next step directly.

### Step 2: Preparation of 5-(trifluoromethyl)-4H-3,1-benzothiazin-2-amine

To a solution of [2-(chloromethyl)-3-(trifluoromethyl)phenyl]ammonium;hydrochloride (16.3 mmol) in (CH₃)₂CHOH (100 ml) was added thiourea (2.5 g, 32.6 mmol) and the resulting reaction mixture was stirred at 80° C. for 16 hours. After completion of the reaction, the pH of the reaction mixture was adjusted to 8 by addition of a saturated aqueous solution of NaHCO₃ at 0° C. The resulting mixture was extracted and the combined organic layers were washed, dried and concentrated to afford a crude product. The crude product was purified by trituration in methyl-tert-butyl ether to afford 5-(trifluoromethyl)-4H-3,1-benzothiazin-2-amine (2.9 g, 78% yield), which was used in next step directly. ¹H-NMR (400 MHz, DMSO-d₆) δ ppm 7.38-7.25 (m, 4H), 7.14 (dd, J=1.8, 7.2 Hz, 1H), 3.97 (s, 2H).

### Step 3: Preparation of 2-[3-ethylsulfonyl-5-(4-fluorophenyl)-2-pyridyl]-6-(trifluoromethyl)-5H-imidazo[1,2-a][3,1]benzothiazine (I.1)

To a solution of 5-(trifluoromethyl)-4H-3,1-benzothiazin-2-amine (2.2 mmol) and 2-bromo-1-[3-ethylsulfonyl-5-(4-fluorophenyl)-2-pyridyl]ethenone (1.7 mmol) in (CH₃)₃COH (100 ml) was added 4 Å molecular sieves (2 g) and the resulting reaction mixture was stirred at 110° C. for 48 hours. After completion of the reaction, the reaction mixture was filtered, and the filtrate was concentrated to afford a crude concentrate. The crude concentrate was purified by trituration in CH₃CN to afford 2-[3-ethylsulfonyl-5-(4-fluorophenyl)-2-pyridyl]-6-(trifluoromethyl)-5H-imidazo[1,2-a][3,1]benzothiazine (166.3 mg, 17% yield). ¹H-NMR (400 MHz, DMSO-d₆) δ ppm 9.20 (d, J=2.1 Hz, 1H), 8.71 (s, 1H), 8.53 (d, J=2.3 Hz, 1H), 8.18 (d, J=8.2 Hz, 1H), 7.93 (dd, J=5.4, 8.8 Hz, 2H), 7.81-7.75 (m, 1H), 7.75-7.66 (m, 1H), 7.41 (t, J=8.8 Hz, 2H), 4.43 (s, 2H), 4.12 (q, J=7.4 Hz, 2H), 1.27 (t, J=7.4 Hz, 3H). LC/MS retention time: 1,699 min, m/z=520 (M+H⁺)

### Synthesis Example 5: Synthesis of [5-ethylsulfonyl-6-[6-(trifluoromethyl)-4H-imidazo[2,1-c][1,4]benzoxazin-2-yl]-3-pyridyl]imino-dimethyl-oxo-lambda6-sulfane (Compound I.5)

### Step-1: Preparation of 2-nitro-6-(trifluoromethyl) phenol

To a stirred solution of 2-(trifluoromethyl)phenol (20.0 g) in dichloroethane (300 mL) was added H₂SO₄ (72.6 g) at 20° C. Then, HNO₃ (11.7 g, 68% purity) was added at 0-5° C. and the resulting mixture was stirred at 5° C. for 1 hour. After the completion of the reaction, the mixture was poured into ice water (1.20 kg ice, 1.80 kg H₂O) with dichloroethane (200 mL). The resulting mixture was allowed to warm to 25° C. with stirring. The aqueous phase was extracted and the combined organic phases concentrated under reduced pressure to give a crude product. The crude product was purified by column chromatography on silica gel to afford 2-nitro-6-(trifluoromethyl)phenol (24.0 g) as a yellow solid. ¹H NMR: (400 MHz, CDCl₃): δ 11.23 (s, 1H), 8.33-8.35 (m, 1H), 7.92-7.94 (m, 1H), 7.10-7.14 (m, 1H).

### Step 2: Preparation of 2-[2-nitro-6-(trifluoromethyl)phenoxy]acetonitrile

To a stirred solution of 2-nitro-6-(trifluoromethyl)phenol (25.0 g) in dimethylformamide (175 mL) was added K₂CO₃ (33.4 g) and 2-chloroacetonitrile (36.4 g) at 25° C. Then, the resulting reaction mixture was stirred at 80° C. for 12 hours. After the completion of the reaction, the reaction mixture was added into H₂O, and extracted. The combined organic phases were washed, dried, filtered and concentrated. The resulting crude product was purified by column chromatography on silica gel to afford 2-[2-nitro-6-(trifluoromethyl)phenoxy]acetonitrile (25.0 g) as a brown oil. ¹H NMR: (400 MHz, CDCl₃): δ 8.19-8.21 (m, 1H), 7.95-7.97 (m, 1H), 7.51-7.55 (m, 1H), 4.93 (s, 2H).

### Step 3: Preparation of 8-(trifluoromethyl)-2H-1,4-benzoxazin-3-amine

A mixture of Fe (15.6 g), NH₄Cl (25.0 g) in CH₃CH₂OH (92.0 mL), H₂O (46.0 mL) was heated to 70° C. Then 2-[2-nitro-6-(trifluoromethyl)phenoxy]acetonitrile (23.0 g) was added to the mixture in portions during a period of 1 hour. Then the resulting reaction mixture was stirred at 80° C. for 1 hour. After the completion the reaction, the internal temperature of the reaction mixture was cooled down to 30° C. EtOAc (60.0 mL) was added to the above reaction mixture, which was subsequently agitated for 30 min. The resulting suspension was filtered, and the filter cake was washed. Organic phases were combined, washed, and concentrated to give a crude product. The crude product was purified by column chromatography on silica gel to give 8-(trifluoromethyl)-2H-1,4-benzoxazin-3-amine (11.7 g) as a white solid. ¹H NMR: (400 MHz DMSO-d₆) δ 7.06-7.10 (m, 3H), 6.95-6.98 (m, 1H), 4.53 (s, 2H).

### Step 4: Preparation of 2-(5-bromo-3-ethylsulfonyl-2-pyridyl)-6-(trifluoromethyl)-4H-imidazo[2,1-c][1,4]benzoxazine

To a stirred solution of 8-(trifluoromethyl)-2H-1,4-benzoxazin-3-amine (1 g) in (CH₃)₃CO (2 V) was added 2-bromo-1-(5-bromo-3-ethylsulfonyl-2-pyridyl)ethanone (1.71 g). A molecular sieve was added to the resulting reaction mixture (1 g), which was subsequently heated to 100° C. for 24 hours. After completion of the reaction, the reaction mixture was filtered. The filtrate was collected and concentrated under reduced pressure to get a crude product. The crude product was purified by column chromatography to afford 2-(5-bromo-3-ethylsulfonyl-2-pyridyl)-6-(trifluoromethyl)-4H-imidazo[2,1-c][1,4]benzoxazine as a yellow solid (0.7 g). LCMS: m/z 488.28, Retention Time: 1.22 min, [M+H] 489.7

### Step 5: Preparation of [5-ethylsulfonyl-6-[6-(trifluoromethyl)-4H-imidazo[2,1-c][1,4]benzoxazin-2-yl]-3-Pyridyl]imino-dimethyl-oxo-lambda6-sulfane (Compound I.5)

To a stirred solution of 2-(5-bromo-3-ethylsulfonyl-2-pyridyl)-6-(trifluoromethyl)-4H-imidazo[2,1-c][1,4]benzoxazine (0.083 g) in 1,4-dioxane (5 V), dimethyl sulfoximine (0.079 g), xanthphos (0.0196 g) and Cs₂CO₃ (0.083 g) were added and the resulting mixture was de-gased under N₂ for 15 minutes. Subsequently, Pd₂(dba)₃ (0.015 g) was added to the mixture, which was then heated to 110° C. in a microwave for 4 hours. After the completion the reaction, the mixture was concentrated to get a crude product. The crude product was purified by column chromatography to afford compound I.5 as a yellow solid (85% yield). LCMS: m/z 500.51, Retention Time: 0.9920 min, [M+H] 501.1

The following examples of formula I-A-A1 were prepared according to, or in analogy to, the above synthesis examples 1-5

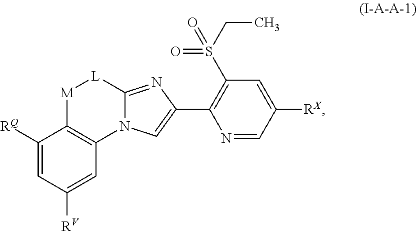

wherein the variables L, M, R^{Q}, R^{V}, and R^{X} have a meaning as defined in Table C:

TABLE C
Compounds of formula (I) that were synthesized according to or
in analogy to the Synthesis Examples 1-5.

| | | | | | | Phys. |
|---|---|---|---|---|---|---|
| | | | | | | Chem. Data*: |
| | | | | | | Retention time |
| | | | | | | [min]; m/z*; |
| | | | | | | HPLC |
| Compound | L | M | R^{Q} | R^{V} | R^{X} | Method |
| I.1 | S | CH₂ | CF₃ | H | 4-fluorophenyl | 1.699; 520; A |
| I.2 | CH₂ | S | CF₃ | H | 4-fluorophenyl | 1.668; 520; A |
| I.3 | O | CH₂ | CF₃ | H | 4-fluorophenyl | 1.646; 504; A |
| I.4 | CH₂ | O | CF₃ | H | 4-fluorophenyl | 1.659; 504; A |
| I.5 | CH₂ | O | CF₃ | H | 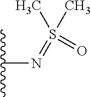 [SMILES: *N=S(C)(C)=O] | 0.992; 501.1; A |
| I.6 | O | CH₂ | CF₃ | H | 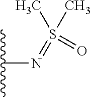 [SMILES: *N=S(C)(C)=O] | 0.973; 501.1; A |
| I.7 | S | CH₂ | OCH₃ | H | Br | 1.425; 467 |
| | | | | | | (M + 2H) |
| I.8 | CH₂ | S | CF₃ | H | 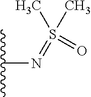 [SMILES: *N=S(C)(C)=O] | 1.017; 517; A |
| I.9 | S | CH₂ | OCF₃ | H | Br | 1.32; 520; C |
| I.10 | CH₂ | S | OCH₃ | H | Br | 1.256; 467 |
| | | | | | | (M + 2H); C |
| I.11 | S | CH₂ | OCH₃ | H | 4-fluorophenyl | 1.29; 482; A |
| I.12 | S | CH₂ | OCH₃ | H | 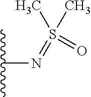 [SMILES: *N=S(C)(C)=O] | 0.920; 479.1; A |
| I.13 | S | CH₂ | OCH₃ | H | 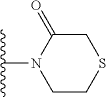 [SMILES: *N1CCSCC1=O] | 1.035; 503.1; A |
| I.14 | CH₂ | O | OCF₃ | H | Br | 2.805; 516.9; B |
| I.15 | S | CH₂ | OCH₃ | H | cyclopropyl | 1.124; 427.8; A |

*mass charge ratio m/z.

### Synthesis Example 6: Synthesis of 2-(5-bromo-3-ethylsulfonyl-2-pyridyl)-1-methyl-6-(trifluoromethoxy)-4H-chromeno[3,4-d]imidazole (Compound I.16)

### Step-1: Synthesis of (1-methylimidazol-4-yl)methanol

To a stirred solution of 1-methylimidazole-4-carboxylic acid (50 g) in THF (320 mL) was added LiAlH₄ (21.1 g) in portions at 0° C., and then the resulting reaction mixture was stirred at 0-20° C. for 16 hours. After the completion of the reaction, the reaction mixture was quenched with addition of Na₂SO₄·10 H₂O (200 g) and subsequently stirred at 20° C. for 1 hour. The resulting mixture was filtered and the filtrate was concentrated to afford a crude solid product. The crude product was purified by trituration with (CH₃OH: CH₂Cl₂=1:10, 1100 mL), and concentrated to give the (1-methylimidazol-4-yl)methanol (41 g, crude) as yellow oil without further purification and used directly.

### Step-2: Preparation of 4-(chloromethyl)-1-methyl-imidazole

To a stirred solution of (1-methylimidazol-4-yl)methanol (31 g) in CH₂Cl₁ (150 mL) was added SOCl₂ (300 mL) dropwise at 0° C. Then, the resulting reaction mixture was stirred at 0-20° C. for 16 hours. After the completion of the reaction, the reaction mixture was concentrated and the resulting residue was washed with CH₂Cl₂, and filtered to give the 4-(chloromethyl)-1-methyl-imidazole (40 g) as brown solid without further purification and used directly.

¹H NMR:(400 MHz, DMSO-d₆) δ=9.19 (s, 1H), 7.78 (s, 1H), 4.89 (s, 2H), 3.85 (s, 3H)

### Step 3: Preparation of 2,5-dibromopyridin-3-amine

To a stirred solution of 2,5-dibromo-3-nitro-pyridine (22.5 g) in CH₃COOH (80 mL) was added Fe (powder, 22.3 g) portions at 10° C., and then the resulting mixture was stirred at 80° C. for 1 hour. After the completion of the reaction, the reaction mixture was quenched with water, extracted, washed, dried and concentrated to give the crude product. The crude procut was purified by column chromatography to give 2,5-dibromopyridin-3-amine (15 g) as yellow solid.

¹H NMR: (400 MHz, CDCl₃) δ=7.81 (d, J=2.3 Hz, 1H), 7.14 (d, J=2.1 Hz, 1H), 4.50-4.18 (brs, 2H)

### Step 4: Preparation of 2,5-dibromo-3-ethylsulfanyl-pyridine

To a sstirred solution of 2,5-dibromopyridin-3-amine (15 g) in dichloroethane: CH₂Cl₂ (75 mL) was added tert-butyl nitrite (9.5 gm) at 20° C., followed by the addition of a solution of diethyl disulfide (11.3 g) in dichloroethane (25 mL), which was added dropwise at 40° C. The resulting mixture was stirred at 40° C. for 2 hours. After the completion of the reaction, the reaction mixture was quenched, extracted, washed, dried, filtered and concentrated. The resulting crude product was purified by column chromatography to afford the 2,5-dibromo-3-ethylsulfanyl-pyridine (12 g) as brown oil.

¹H NMR: (400 MHz, CDCl₃) δ=8.17 (d, J=2.1 Hz, 1H), 7.50 (d, J=2.3 Hz, 1H), 2.96 (q, J=7.4 Hz, 2H), 1.42 (t, J=7.4 Hz, 3H).

### Step 5: Preparation of (5-bromo-3-ethylsulfanyl-2-pyridyl)-tributyl-stannane

To a stirred solution of 2,5-dibromo-3-ethylsulfanyl-pyridine (2 g) in toluene (25 mL) was added n-butyl lithium (8.1 mmol) dropwise at −65° C. The resulting mixture was stirred at −65° C. for 1 hour under N₂, followed by the addition of ClSn(Bu)₃ (2.6 g), which was added dropwise at −65° C. The resulting mixture was stirred at −65° C. for 16 hours under N₂. After the completion of the reaction, the reaction mixture was quenched, extracted, washed, dried, filtered and concentrated. The resulting crude product was purified by column chromatography to afford the (5-bromo-3-ethylsulfanyl-2-pyridyl)-tributyl-stannane (1.8 g).

¹H NMR: (400 MHz, DMSO-d₆) δ=8.61 (s, 1H), 7.93 (s, 1H), 3.05 (q, J=7.3 Hz, 2H), 1.51 (m, 6H), 1.59-1.32 (m, 6H), 1.31-1.17 (m, 3H), 1.15-1.08 (m, 6H), 0.83 (t, J=7.3 Hz, 9H).

### Step 6: Preparation of 1-(methoxymethoxy)-2-(trifluoromethoxy)benzene

To a stirred solution of 2-(trifluoromethoxy)phenol (27.5 g) and diethylamine (49.8 g) in CH₂Cl₂ (300 mL) was added chloromethyl methyl ether (31 g) at 0° C. The resulting composition was stirred at 0° C. for 0.5 hours. After the completion of the reaction starting material, two parallel reactions were combined and work up together. The reaction mixture was quenched into water, extracted, washed, dried, filtered and concentrated to give the 1-(methoxymethoxy)-2-(trifluoromethoxy)benzene (75 g, crude) as yellow oil without further purification and used directly. ¹H NMR: (400 MHz, CDCl₃) δ=7.31-7.25 (m, 3H), 7.08-6.98 (m, 1H), 5.25 (s, 2H), 3.53 (s, 3H)

### Step 7: Preparation of 1-bromo-2-(methoxymethoxy)-3-(trifluoromethoxy)benzene

To a stirred solution of 1-(methoxymethoxy)-2-(trifluoromethoxy)benzene (37.5 g) in THF (500 mL) was added n-butyl lithium (253.4 mmol) at −65° C. The resulting composition was stirred at −65° C. for 0.5 hours. Then, dibromotetrafluoroethane (65.4 g) in THF (100 mL) was added to the mixture dropwise, which was subsequently stirred at −65° C. for 1 hour. After the completion of the reaction, two parallel reactions were combined and worked up together: The reaction mixture was quenched, extracted, washed, dried, filtered and concentrated to afford a crude product. The crude product was purified by column chromatography to give the 1-bromo-2-(methoxymethoxy)-3-(trifluoromethoxy)benzene (66.8 g) as yellow oil. ¹H NMR: (400 MHz, CDCl₃) δ=7.52 (dd, J=1.4, 8.1 Hz, 1H), 7.25 (td, J=1.4, 8.3 Hz, 1H), 7.03 (t, J=8.2 Hz, 1H), 5.18 (s, 2H), 3.67 (s, 3H)

### Step 8: Preparation of 2-bromo-6-(trifluoromethoxy)phenol

To a stirred solution of 1-bromo-2-(methoxymethoxy)-3-(trifluoromethoxy)benzene (33.4 g) in CH₃OH (300 mL) was added toluene sulfonic acid (38.1 g) at 20° C. The resulting composition was stirred at 20° C. for 3 hours. After the completion of the reaction, two parallel reactions were combined and worked up together. The reaction mixture was quenched, extracted, washed, dried, filtered and concentrated to give the 2-bromo-6-(trifluoromethoxy)phenol (75 g, crude) as yellow oil without further purification and used directly. ¹H NMR: (400 MHz, CDCl₃) δ=7.43 (dd, J=1.4, 8.1 Hz, 1H), 7.20 (td, J=1.4, 8.3 Hz, 1H), 7.02-7.02 (m, 1H), 6.24 (brs, 1H).

### Step 9: Preparation of 4-[[2-bromo-6-(trifluoromethoxy)phenoxy]methyl]-1-methyl-imidazole

A composition was prepared comprising 2-bromo-6-(trifluoromethoxy)phenol (37.5 g), 4-(chloromethyl)-1-methyl-imidazole (6.5 g), and Cs₂CO₃ (38 g) in dimethylformamide (100 mL), which composition was stirred at 20° C. for 16 hours. After the completion of the reaction, two parallel reactions were combined and worked up together. The reaction mixture was quenched, extracted, washed, dried, filtered and concentrated tp afford a crude product. The crude product was purified by column chromatographyto give the 4-[[2-bromo-6-(trifluoromethoxy)phenoxy]methyl]-1-methyl-imidazole (12 g) as yellow oil. ¹H NMR: (400 MHz, CDCl₃) δ=7.53-7.44 (m, 2H), 7.23 (d, J=8.2 Hz, 1H), 7.07 (s, 1H), 7.01 (t, J=8.2 Hz, 1H), 5.07 (s, 2H), 3.71 (s, 3H)

### Step 10: Preparation of 1-methyl-6-(trifluoromethoxy)-4H-chromeno[3,4-d]imidazole

A composition was prepared comprising 4-[[2-bromo-6-(trifluoromethoxy)phenoxy]methyl]-1-methyl-imidazole (5.5 g), K(CH₃COO) (6.1 g), Pd(CH₃COO)₂ (706 mg) and triphenylphosphan (1.6 g) in DMSO (60 mL) at 20° C. The composition was then stirred at 100° C. for 16 hours under N₂. After the completion of the reaction, two parallel reactions were combined and worked up together. The reaction mixture was quenched, extracted, washed, dried, filtered and concentrated to afford a crude product. The crude product was purified by column chromatography to afford 1-methyl-6-(trifluoromethoxy)-4H-chromeno[3,4-d]imidazole (5 g) as a brown solid. ¹H NMR: (400 MHz, CDCl₃) δ=7.44 (s, 1H), 7.31 (dd, J=1.3, 7.8 Hz, 1H), 7.08 (d, J=8.2 Hz, 1H), 6.99-6.90 (m, 1H), 5.40 (s, 2H), 3.92 (s, 3H).

### Step 11: Preparation of 2-bromo-1-methyl-6-(trifluoromethoxy)-4H-chromeno[3,4-d]imidazole

A composition was prepared containing 1-methyl-6-(trifluoromethoxy)-4H-chromeno[3,4-d]imidazole (2.5 g) in dimethylformamide (50 mL). N-bromosuccinimide (2.5 g) was added to the compositon at 20° C., which composition was subsequently stirred at 20° C. for 3 hours. After the completion of the reaction, two parallel reactions were combined and worked up together. The reaction mixture was quenched, extracted, washed, dried, filtered and concentrated to afford a crude product. The crude product was purified by trituration ((petrol ether:EtOAc=5:1) to afford 2-bromo-1-methyl-6-(trifluoromethoxy)-4H-chromeno[3,4-d]imidazole (2.3 g) as yellow solid. ¹H NMR: (400 MHz, CDCl₃) δ=7.32 (dd, J=1.3, 7.8 Hz, 1H), 7.12 (td, J=1.2, 8.3 Hz, 1H), 7.01-6.94 (m, 1H), 5.34 (s, 2H), 3.90 (s, 3H)

### Step 12: Preparation of 2-(5-bromo-3-ethylsulfanyl-2-pyridyl)-1-methyl-6-(trifluoromethoxy)-4H-chromeno[3,4-d]imidazole

To a stirred solution of 2-bromo-1-methyl-6-(trifluoromethoxy)-4H-chromeno[3,4-d]imidazole (1.06 g) and (5-bromo-3-ethylsulfanyl-2-pyridyl)-tributyl-stannane (2.6 g) in toluene (60 mL) was added [1,1′-Bis-(diphenylphosphino)-ferrocen]-dichloro-palladium(II) (450 mg) and CuI (152 mg) at 20° C. The resultinc reaction mixture was then stirred at 120° C. for 16 hours. under N₂. After the completion of the reaction, the reaction mixture was quenched, extracted, washed, dried, filtered and concentrated to afford a crude product. The crude product was purified by column chromatography to afford 2-(5-bromo-3-ethylsulfanyl-2-pyridyl)-1-methyl-6-(trifluoromethoxy)-4H-chromeno[3,4-d]imidazole (800 mg) as yellow solid. ¹H NMR: (400 MHz, CDCl₃) δ=8.49 (d, J=2.0 Hz, 1H), 7.79 (d, J=2.0 Hz, 1H), 7.43 (dd, J=1.4, 7.8 Hz, 1H), 7.17-7.07 (m, 1H), 6.99 (t, J=8.0 Hz, 1H), 5.49 (s, 2H), 3.99 (s, 3H), 3.00-2.92 (m, 2H), 1.39 (t, J=7.4 Hz, 3H).

### Step 13: Preparation of 2-(5-bromo-3-ethylsulfonyl-2-pyridyl)-1-methyl-6-(trifluoromethoxy)-4H-chromeno[3,4-d]imidazole

To a solution of 2-(5-bromo-3-ethylsulfanyl-2-pyridyl)-1-methyl-6-(trifluoromethoxy)-4H-chromeno[3,4-d]imidazole (800 mg) in CH₂Cl₂ (10 mL) was added meta-chloroperoxybenzoic acid (839 mg) at 0° C. The resulting reaction mixture was stirred at 0° C. for 1 hour. After the completion of the reaction, the reaction mixture was quenched, extracted, washed, dried, filtered and concentrated of afford a crude product. The crude product was purified HPLC to afford compound I.16 (200 mg, 24.1%) as a white solid. ¹H NMR: (400 MHz, CDCl₃) δ=9.00 (d, J=2.1 Hz, 1H), 8.62 (d, J=2.1 Hz, 1H), 7.42 (dd, J=1.4, 7.9 Hz, 1H), 7.19-7.14 (m, 1H), 7.05-6.99 (m, 1H), 5.45 (s, 2H), 3.89-3.81 (m, 5H), 1.38 (t, J=7.4 Hz, 3H).

### B. BIOLOGICAL EXAMPLES

The activity of the compounds of formula (I) of the present invention could be demonstrated and evaluated in biological tests described in the following. If not otherwise specified, the test solutions are prepared as follows: The active compound is dissolved at the desired concentration in a mixture of 1:1 (vol:vol) distilled water:acteone. The test solution is prepared at the day of use. Test solutions are prepared in general at concentrations of 1000 ppm, 500 ppm, 300 ppm, 100 ppm, 80 ppm, and 30 ppm (wt/vol).

### Yellow Fever Mosquito (Aedes aegypti)

For evaluating control of yellow fever mosquito (Aedes aegypti) the test unit consisted of 96-well-microtiter plates containing 200 μl of tap water per well and 5-15 freshly hatched A. aegypti larvae. The active compounds were formulated using a solution containing 75% (v/v) water and 25% (v/v) DMSO. Different concentrations of formulated compounds or mixtures were sprayed onto the insect diet at 2.5 μl, using a custom built micro atomizer, at two replications. After application, microtiter plates were incubated at 28+1° C., 80+5% RH for 2 days. Larval mortality was then visually assessed. In this test, compounds I.1, I.2, I.3, and I.4 at 80 ppm showed over 70% mortality in comparison with untreated controls. In this test, compounds I.5, I.6 at 250 ppm showed over 70% mortality in comparison with untreated controls.

### Boll Weevil (Anthonomus grandis)

For evaluating control of boll weevil (Anthonomus grandis) the test unit consisted of 96-well-microtiter plates containing an insect diet and 5-10 A. grandis eggs. The compounds were formulated using a solution containing 75% v/v water and 25% v/v DMSO. Different concentrations of formulated compounds were sprayed onto the insect diet at 5 μl, using a custom built micro atomizer, at two replications. After application, microtiter plates were incubated at about 25±1° C. and about 75±5% relative humidity for 5 days. Egg and larval mortality was then visually assessed. In this test, compounds I.1, I.2, I.3 and I.4 at 10 ppm showed over 70% mortality in comparison with untreated controls. In this test, compounds I.5 and I.6 at 250 ppm showed over 70% mortality in comparison with untreated controls.

### Tobacco Budworm (Heliothis virescens)

For evaluating control of tobacco budworm (Heliothis virescens) the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom built micro atomizer, at two replications. After application, microtiter plates were incubated at about 28±1° C. and about 80±5% relative humidity for 5 days. Egg and larval mortality was then visually assessed. In this test, compounds I.1, I.2, I.3, and I.4 at 30 ppm showed over 70% mortality in comparison with untreated controls.

### Green Peach Aphid (Myzus persicae)

The active compounds were formulated by a Tecan liquid handler in 100% cyclohexanone as a 10,000 ppm solution supplied in tubes. The 10,000 ppm solution was serially diluted in 100% cyclohexanone to make interim solutions. These served as stock solutions for which final dilutions were made by the Tecan in 50% acetone:50% water (v/v) into 10or 20 ml glass vials. A nonionic surfactant (Kinetic®) was included in the solution at a volume of 0.01% (v/v). The vials were then inserted into an automated electrostatic sprayer equipped with an atomizing nozzle for application to plants/insects. Bell pepper plants at the first true-leaf stage were infested prior to treatment by placing heavily infested leaves from the main colony on top of the treatment plants. Aphids were allowed to transfer overnight to accomplish an infestation of 30-50 aphids per plant and the host leaves were removed. The infested plants were then sprayed by an automated electrostatic plant sprayer equipped with an atomizing spray nozzle. The plants were dried in the sprayer fume hood, removed, and then maintained in a growth room under fluorescent lighting in a 14:10 light:dark photoperiod at about 25° C. and about 20-40% relative humidity. Aphid mortality on the treated plants, relative to mortality on untreated control plants, was determined after 5 days. In this test, compounds I.2 and I.3 at 90 ppm showed over 70% mortality in comparison with untreated controls. In this test, compounds I.5 and I.6 at 250 ppm showed over 70% mortality in comparison with untreated controls.

### Red Spider Mite (Tetranychus kanzawai)

The active compound is dissolved at the desired concentration in a mixture of 1:1 (vol:vol) distilled water:acetone. Add surfactant (Kinetic) is added at a rate of 0.01% (vol/vol). The test solution is prepared at the day of use. Potted cowpea beans of 4-5 days of age are cleaned with tap water and sprayed with 1-2 ml of the test solution using air driven DeVilbiss® hand atomizer at 20-30 psi (≈1.38 to 2.07 bar). The treated plants are allowed to air dry and afterwards inoculated with 30 or more mites by clipping a cassava leaf section from rearing population. Treated plants are placed inside a holding room at about 25-26° C. and about 65-70% relative humidity. Estimate percent mortality is assessed 72 hours after treatment. In this test, compound I.3 at 800 ppm showed over 70% mortality in comparison with untreated controls.

### Southern Armyworm (Spodoptera eridania), 2nd Instar Larvae

The active compounds were formulated by a Tecan liquid handler in 100% cyclohexanone as a 10,000 ppm solution supplied in tubes. The 10,000 ppm solution was serially diluted in 100% cyclohexanone to make interim solutions. These served as stock solutions for which final dilutions were made by the Tecan in 50% acetone:50% water (v/v) into 10 or 20 ml glass vials. A nonionic surfactant (Kinetic®) was included in the solution at a volume of 0.01% (v/v). The vials were then inserted into an automated electrostatic sprayer equipped with an atomizing nozzle for application to plants/insects. Lima bean plants (variety Sieva) were grown 2 plants to a pot and selected for treatment at the 1^{st} true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. Ten to 11 armyworm larvae were placed into the bag and the bags zipped closed. Test plants were maintained in a growth room at about 25° C. and about 20-40% relative humidity for 4 days, avoiding direct exposure to fluorescent light (14:10 light:dark photoperiod) to prevent trapping of heat inside the bags. Mortality and reduced feeding were assessed 4 days after treatment, compared to untreated control plants. In this test, compounds I.1, I.2, I.3, and I.4 at 1 ppm showed over 70% mortality in comparison with untreated controls. In this test, compound I.9 at 300 ppm showed over 70% mortality in comparison with untreated controls.

1. A compound of formula (I), or an agrochemically or veterinarily acceptable salt, stereoisomer, tautomer, or N-oxide thereof 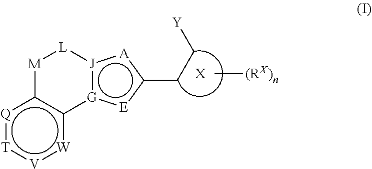
wherein the variables in formula (I) have the following meaning
A is CH, N, or NH;
E is N, O, S, NR^{E}, or CR^{E};
G, J are independently C or N, provided that only one of E or G is N;
the group -M-L- is O—CR^{L1}R^{L2}, S—CR^{L1}R^{L2}, CR^{M1}R^{M2}—O, or CR^{M1}R^{M2}—S;
Q is N or CR^{Q};
T is N or CR^{T};
V is N or CR^{v};
W is N or CR^{W};
X is phenyl or a 5- or 6-membered heteroaryl;
Y is SR^{Y1}, S(O)R^{Y1}, S(O)₂R^{Y1}, S(═O)(═NR^{Y2})R^{Y1}, or
S(═NR^{Y2})(═NR^{Y3})R^{Y1};
R^{E}, R^{Q}, R^{T}, R^{V}, and R^{W} are independently H, halogen, N₃, CN, NO₂, SCN, SF₅; C₁-C₆-alkyl, C₁-C₆-alkoxy, C₂-C₆-alkenyl, tri-C₁-C₆-alkysilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxyx-C₁-C₄-alkyl, which groups are halogenated or non-halogenated,
C(═O)OR¹, NR²R³, C(═O)NR²R³, C(═O)R⁴, SO₂NR²R³, S(═O)ₘR⁵, OR⁶, SR⁶, or CH₂R⁶;
phenyl, which is unsubstituted or substituted with one or more, same or different substituents R¹¹;
R¹ H;
C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆ -cycloalkoxy-C₁-C₄-alkyl, which groups are halogenated or non-halogenated; or
phenyl, which is unsubstituted or substituted with one or more, same or different substituents R¹¹;
R¹¹ is halogen, OH, CN, NO₂, SCN, SF₅;
C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆ -cycloalkyl, C₃-C₆ -cycloalkoxy, C₃-C₆-cycloalkyl-C₁-C₄-alkyl, C₃-C₆-cycloalkoxy-C₁-C₄-alkyl, which groups are halogenated or non-halogenated;
R² is H;
C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with one or more, same or different substitutent selected from halogen, CN, and HO.
C(═O)R²¹, C(═O)OR²¹, C(═O)NR²¹, C₁-C₆-alkylen-CN, or CH₂R⁶; or
phenyl, which is unsubstituted or substituted with one or more, same or different substituents R¹¹;
R²¹ is H;
C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl; C₃-C₆-cycloalkyl-C₁-C₄-alkyl, C₃-C₆-cycloalkoxy-C₁-C₄alkyl, phenyl, or a saturated, partially-, or fully unsaturated 5- or 6-membered heterocycle, wherein the cyclic moieties are unsubstituted or substituted with one or more, same or different substituents R¹¹;
R³ is H;
C₁-C₆-alkyl, C₂-C₆-alkenyl, C₂-C₆-alkynyl, C₁-C₆-aloxy-C₁-C₄-alkyl, C₃-C₆-cycloalkyl, C₃-C₆-cycloalkoxy-C₁-C₄-alkyl, C₃-C₆-cycloalkoxy-C₁-C₄-alkyl, which groups are halogenated or non-halogenated;
C₁-C₆-alkylen-CN, or CH₂R⁶;
phenyl, which is unsubstituted or substituted with one or more, same or different substituents R¹¹; or
NR²R³ optionally can form an N-bound, saturated 3- to 8-membered heterocycle, which in addition to the nitrogen atom may have 1 or 2 further heteroatoms or heteroatom moieties selected from O, S(═O)ₘ, NH, and N—C₁-C₆-alkyl, and wherein the N-bound heterocycle is unsubstituted or substituted with one or more, same or different substituents selected from halogen, C₁-C₄-alkyl, C₁-C₄-haloalkyl, C₁-C₄-alkoxy, and C₁-C₄-haloalkoxy;
R⁴ is H, C₁-C₄-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆-cycloalkoxy-C₁-C₄-alkyl, which are unsubstituted or substituted with one or more, same of different substituents selected from halogen, CN, and OH;
CH₂R⁶, or phenyl, which is unsubstituted or substituted with one or more, same or different substituents R¹¹;
R⁵ is C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆-cycloalkoxy-C₁-C₄-alkyl, which groups are halogenated or non-halogenated;
C₁-C₆-alkylen-NR²R³, C₁-C₆-alkylen-CN, CH₂R⁶; or
phenyl, which is unsubstituted or substituted with one or more, same or different substituents R¹¹;
R⁶ is phenyl, which is unsubstituted or substituted with one or more, same or different substituents R¹¹;
each R^{L1}, R^{L2}, R^{M1}, R^{M2} is independently H, halogen, OH;
C₁-C₄-alkyl, C₃-C₆-cycloalkyl, C₃-C₆-cycloalkyl-C₁-C₂-alkyl, C₂-C₄-alkenyl, C₃-C₆-cycloalkenyl, C₂-C₄-alkynyl, C₁-C₄-alkoxy, which groups are unsubstituted, or substituted with one or more, same or different substituents selected from halogen and CN,
phenyl, which is unsubstituted or substituted with one or more, same or different substituents selected from halogen, CN, C₁-C₃-alkyl, C₁-C₃-haloalkyl, C₁-C₃-alkoxy, and C₁-C₃-haloalkoxy; or
R^{L1} and R^{L2}, or R^{M1}and R^{M2} form together with the carbon atom they are bonded to a group C═O, C═S, C═NR⁷R⁸, or C═NOR⁷;
each R⁷, R⁸ is independently H;
C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with one or more, same or different substitutent selected from halogen, CN and OH.
benzyl or phenyl, which is unsubstituted or substituted with one or more, same or different substituents R¹¹, or
NR⁷R⁸ may also form an N-bound, saturated 3- to 6-membered heterocycle, which in addition to the nitrogen atom may have 1 or 2 further heteroatoms or heteroatom moieties selected from O, S(═O)ₘ, NH, and N—C₁-C₆-alkyl, and wherein the N-bound heterocycle is unsubstituted or substituted with one or more, same or different substituents selected from halogen, C₁-C₄-alkyl, C₁-C₄-haloalkyl, C₁-C₄-alkoxy and C₁-C₄-haloalkoxy;
each R^{X} is independently halogen, N₃, OH, CN, NO₂, SCN, SF₅;
C₁-C₆-alkyl, C₁-C₆-alkoxy, C₂-C₆-alkenyl, tri-C₁-C₆-alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, which groups are unsubstituted or substituted with CN or halogen;
C(═O)OR¹, NR²R³, C(═O)NR²R³, C(═O)R⁴, SO₂NR²R³, S(═O),R¹, OR⁶, SR⁶, CH₂R⁶, OC(═O)R⁴, NR³C(═O)R⁴, OC(═O)OR¹, OC(═O)NR²R³, OC(═O)SR¹, OC(═S)NR²R³, OC(═S)SR¹, ONR²R³, ON=CR¹R⁴, N═CR¹R⁴, NNR², NC(═O)R⁹, SC(═O)SR¹, SC(═O)NR²R³, C(═S)R⁶, C(═S)OR⁴, C(═NR²)R⁴, C(R^{10a})═NO(R^{10b});
phenyl, which is unsubstituted or substituted with one or more, same or different substituents R¹¹;
a 5- or 6-membered saturated, partially unsaturated, or fully unsaturated heterocyclic ring, wherein said heterocyclic ring comprises one or more, same or different heteroatoms O, N, or S, and is unsubstituted or substituted with one or more, same or different substituents R³¹, and wherein said N- and S-atoms are independently oxidized, or non-oxidized; or
a group of formula (S) 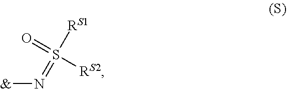
wherein each R^{S1}, RS² is independently selected from C₁-C₆-alkyl, C₃-C₆-cycloalkyl, C₂-C₆-alkenyl, C₃-C₆-cycloalkenyl, C₂-C₆-alkynyl, C₃-C₆-cycloalkyl-C₁-C₃-alkyl, which groups are unsubstituted or halogenated;
a 3- to 6-membered saturated, partially unsaturated, or fully unsaturated heterocyclic ring or ring system, wherein said heterocyclic ring or ring system comprises one or more, same or different heteroatoms O, N, or S, and is unsubstituted or substituted with one or more, same or different substituents selected from halogen, C₁-C₃-alkyl, C₁-C₃-alkoxy, C₁-C₃-haloalkyl, and C₁-C₃-haloalkoxy, and wherein said N- and S-atoms are independently oxidized or non-oxidized;
phenyl, which is unsubstituted or substituted with one or more, same or different substituents selected from halogen, C₁-C₃-alkyl, C₁-C₃-alkoxy, C₁-C₃-haloalkyl, and C₁-C₃-haloalkoxy;
or two substituents R^{S1}, R^{S2} form, together with the sulfur atom to which they are bound, a 5- or 6- membered saturated, partially unsaturated, or fully unsaturated heterocycle, which heterocycle is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said heterocycle comprises no, one or more, same or different heteroatoms O, N, or S in addition to the sulfur atom to which R^{S1} and R^{S2} are bound to;
R³¹ is halogen, N₃, OH, CN, NO₂, SCN, SF₅;
C₁-C₆-alkyl, C₁-C₆-haloalkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₁-C₆-alkoxycarbonyl, C₃-C₆-cycloalkyl;
C₃-C₆-cycloalkoxy, C₃-C₆-cycloalkyl-C₁-C₄-alkyl, C₃-C₆-cycloalkoxy-C₁-C₄ alkyl, phenyl, or a saturated, partially-, or fully unsaturated 5- or 6-membered heterocycle, wherein the cyclic moieties are unsubstituted or substituted with one or more, same or different substituents R¹¹; or
two geminal substituents R³¹ form together with the atom to which they are bound a group ═O or ═S;
each R⁹ is independently C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with one or more, same or different substituents selected from CN and halogen;
each R^{10a} is independently H, CN, OH, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen;
phenyl or benzyl, wherein the phenyl ring is unsubstituted or substituted with one or more, same or different substituents R¹¹;
each R^{10b} is independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with one or more, same or different substituents selected from halogen and CN;
phenyl or benzyl, wherein the phenyl ring is unsubstituted or substituted with one or more, same or different substitutents R¹¹;
each R^{Y1}, R^{Y2}, R^{Y3} is independently selected from H, C₁-C₆-alkyl, C₃-C₆-cycloalkyl, C₂-C₆-alkenyl, C₃-C₆-cycloalkenyl, C₂-C₆-alkynyl, C₃-C₆-cycloalkyl-C₁-C₃-alkyl, which groups are unsubstituted or substituted with one or more, same or different substituents selected from halogen and CN;
a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted or substituted with one or more, same or different substituents selected from halogen, CN, C₁-C₃-alkyl, C₁-C₃-alkoxy, C₁-C₃-haloalkyl, C₁-C₃-haloalkoxy, and wherein said N- and S-atoms are independently oxidized or non-oxidized;
phenyl, which is unsubstituted, or substituted with one or more, same or different substituents selected from halogen, CN, C₁-C₃-alkyl, C₁-C₃-alkoxy, C₁-C₃-haloalkyl, C₁-C₃-haloalkoxy;
or two substituents selected from R^{Y1}, R^{Y2}, R^{Y3}, form, together with the S- or N-atoms to which they are bound, a 5- or 6-membered saturated, partially unsaturated, or fully unsaturated heterocycle, which heterocycle is unsubstituted, or substituted with one or more, same or different substituents selected from halogen, CN, C₁-C₃-alkyl, C₁-C₃-alkoxy, C₁-C₃-haloalkyl, C₁-C₃-haloalkox, and wherein said heterocycle comprises no, one or more, same or different heteroatoms O, N, or S in addition to the S- and N-atoms to which the two substituents selected from R^{Y1}, R^{Y2}, and R^{3Y}, are bound to;
the index n is 0, 1, 2, 3, or 4 if X is phenyl or a 6-membered heteroaryl;
or 0, 1, 2, or 3 if X is a 5-membered heteroaryl; and
the index m is 0, 1, or 2.

2. The compound of formula (I) according to claim 1, wherein A is N.

3. The compound of formula (I) according to claim 1, wherein X is phenyl or 2-pyridyl.

4. The compound of formula (I) according to claim 1, wherein R^{Q}, R^{T}, R^{V}, and R^{W} are independently H; or
C₁-C₃-alkyl, C₁-C₃-alkoxy, which groups are halogenated or non-halogenated.

5. The compound of formula (I) according to claim 1, wherein R^{Y1} is C₁-C₃-alkyl, which is unsubstituted or halogenated.

6. The compound of formula (I) according to claim 1, wherein
R^{E} is H;
C₁-C₃-alkyl, C₁-C₃-alkoxy, which groups are unsubstituted or halogenated.

7. The compound of formula (I) according to claim 1, wherein
each R^{L1}, R^{L2}, R^{M1}, R^{M2} is independently H, C₁-C₃-alkyl, C₁-C₃-haloalkyl; or
R^{L1} and R^{L2}, or R^{M1} and R^{M2} form together with the carbon atom they are bonded to a group C═O, or C═S.

8. The compound of formula (I) according to claim 1, wherein
each R^{X} is independently halogen;
C₁-C₃-alkyl, C₁-C₃-alkoxy, which groups are unsubstituted or substituted with CN or halogen;
NR³C(═O)R⁹, C(R^{10a})═N—O(R^{10b});
phenyl, which is unsubstituted or halogenated;
a 5- or 6-membered saturated, partially unsaturated, or fully unsaturated heterocyclic ring, wherein said heterocyclic ring comprises one or more, same or different heteroatoms O, N, or S, and is unsubstituted or substituted with one or more, same or different substituents selected from halogen, CN, C₁-C₃-alkyl, or C₁-C₃-haloalkyl, and wherein two substituents may form together with the carbon-atom to which they are bound a group (C═O), and wherein said N- and S-atoms are independently oxidized or non-oxidized; or
a group of formula (S) 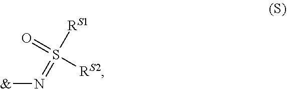
wherein each R^{S1}, R^{S2} is independently selected from C₁-C₃-alkyl, which groups are unsubstituted, or halogenated;
or two substituents R^{S1}, R^{S2} form, together with the sulfur atom to which they are bound, a 5- or 6- membered saturated, partially unsaturated, or fully unsaturated heterocycle, which heterocycle is unsubstituted or substituted with one or more, same or different substituents selected from halogen, C₁-C₃-alkyl, C₁-C₃-alkoxy, C₁-C₃-haloalkyl, and C₁-C₃-haloalkoxy, and wherein said heterocycle comprises no, one or more, same or different heteroatoms O, N, or S in addition to the sulfur atom to which R^{S1} and R^{S2} are bound to.

9. The compound of formula (I) as defined in claim 1, wherein the compound of formula (I) is selected from formula (II), (III), or (IV) 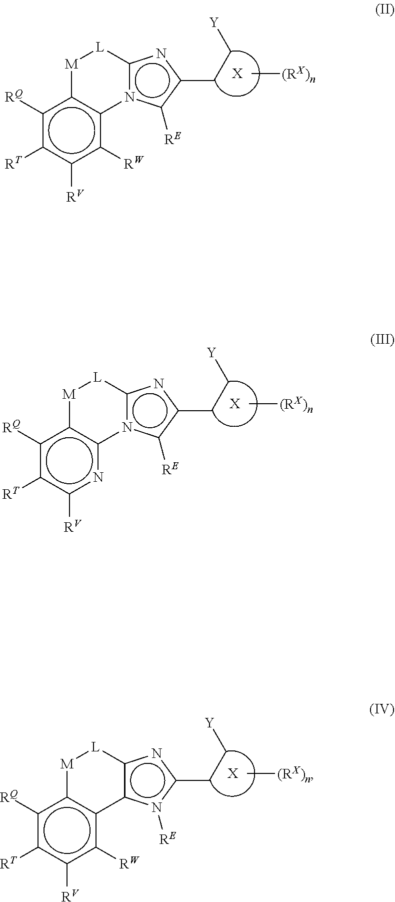
wherein all variables are defined as for formula (I).

10. (canceled)

11. A pesticidal mixture comprising a compound of formula (I) as defined in claim 1, and another agrochemically active ingredient.

12. An agrochenmical or veterinary composition comprising a compound of formula (I) as defined in claim 1, and a liquid or solid carrier.

13. A method for controlling invertebrate pests, infestation, or infection by invertebrate pests, comprising contacting the invertebrate pests, their food supply, habitat, breeding grounds, or their locus with a compound of formula (I) as defined in claim 1, in a pesticidally effective amount.

14. A seed, comprising a compound of formula (I) as defined in claim 1 in an amount of from 0.1 g to 10 kg per 100 kg of the seed.

15. The pesticide mixture of claim 11 wherein the another agrochemically active ingredient is a pesticide.

16. The pesticide mixture of claim 15 wherein the pesticide is an insecticide and/or a fungicide.
